(19) United States
(12) Patent Application Publication
(10) Pub. No.: US 2025/0276952 A1
(43) Pub. Date: Sep. 4, 2025

(54) 4-AMINOPHENYL-THIAZOLE DERIVATIVES AND METHODS OF USE THEREOF

(71) Applicant: CYTEIR THERAPEUTICS, INC., Lexington, MA (US)

(72) Inventors: Matthew Alan BELMONTE, Hollis, NH (US); William D. BRADLEY, Belmont, MA (US); Jean-Marc LAPIERRE, Merrimack, NH (US); Casey Cameron MCCOMAS, Phoenixville, PA (US); John Paul SECRIST, Southborough, MA (US); Joseph VACCA, Telford, PA (US)

(21) Appl. No.: 18/859,622

(22) PCT Filed: Apr. 25, 2023
(86) PCT No.: PCT/US2023/019817

§ 371 Date: Oct. 24, 2024

Related U.S. Application Data
(60) Provisional application No. 63/334,589, filed on Apr. 25, 2022.

(51) Int. Cl. C07D 277/28; A61K 31/4178; A61K 31/426; A61K 31/4436; C07D 233/58; C07D 417/12

(52) U.S. Cl. CPC C07D 277/28; A61K 31/4178; A61K 31/426; A61K 31/4436; C07D 233/58; C07D 417/12

(57) ABSTRACT

The present disclosure relates to compounds of Formula (I):

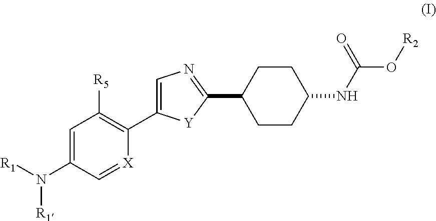

and to their pharmaceutically acceptable prodrugs, solvates, or salts, pharmaceutical compositions, methods of use, and methods for their preparation. The compounds disclosed herein are useful for the treatment of disorders such as a cancer or a neurodegenerative disease.

### CROSS-REFERENCES TO RELATED APPLICATIONS

This application claims priority to, and the benefit of, U.S. Provisional Application No. 63/334,589, filed on Apr. 25, 2022, the entire contents of which are incorporated herein by reference.

### BACKGROUND

Monocarboxylate transporters (MCTs) mediate influx and efflux of monocarboxylates such as lactate, pyruvate, ketone bodies (acetoacetate and beta-hydroxybutyrate) across cell membranes. These monocarboxylates play an essential role in carbohydrate, amino acid, and fat metabolism in mammalian cells. MCTs catalyze the transport of solutes via a facilitative diffusion mechanism that requires co-transport of protons. Monocarboxylates such as lactate, pyruvate, and ketone bodies play a central role in cellular metabolism and metabolic communications among tissues. Lactate is the end product of aerobic glycolysis. Lactate has recently emerged as a critical regulator of cancer development, invasion, and metastasis. Tumor lactate levels correlate well with metastasis, tumor recurrence, and poor prognosis.

MCTs are 12-span transmembrane proteins with cytosolic N- and C-termini, and are members of solute carrier SLC16A gene family. MCT family contains 14 members (e.g., MCT1, MCT2, MCT3, and MCT4 perform the function of transporting lactate, pyruvate, and ketone bodies).

Malignant tumors contain well oxygenated and hypoxic regions, and this hypoxia is associated with increased risk of cancer invasion and metastasis. Tumor hypoxia is associated with treatment failure, relapse, and patient mortality as these hypoxic cells are generally resistant to standard chemotherapy and radiation therapy. In tumors, cancer cells often prefer to utilize glycolysis rather than oxidative phosphorylation to generate energy by metabolizing glucose into lactate, and are thus referred to as glycolytic tumors. In order to avoid lactate-induced cytotoxicity, glycolytic cancer cells upregulate the expression of MCTs to increase their export capacity and avoid reaching toxic intracellular levels of lactate. In addition, nearby cancer cells have been shown to consume this lactate via MCT1 and utilize it for energy production in place of glucose.

The disclosure arises from a need to provide further compounds for the modulation of monocarboxylate transporters (MCTs). In particular, compounds with improved physicochemical, pharmacological and pharmaceutical properties to existing compounds are desirable.

### SUMMARY

In some aspects, the present disclosure relates to a compound of Formula (I):

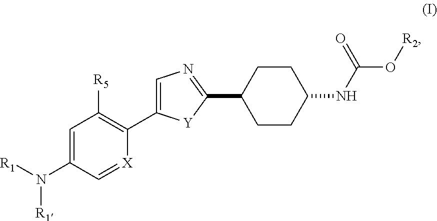

or a pharmaceutically acceptable prodrug, solvate, or salt thereof, wherein:
X is CR₃ or N;
Y is S or NR₄;
R₁ is H, —C(O)—R₁ₐ, —C(NH)—R₁ₐ, C₁-C₆ alkyl, C₂-C₆ alkenyl, C₂-C₆ alkynyl, C₆-C₁₀ aryl, or 5- to 10-membered heteroaryl;
R_{1′} is H or C₁-C₆ alkyl;
R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl, wherein the —O(C₁-C₆ alkyl), —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl is optionally substituted with one or more Rib;
R_{1b} is C₆-C₁₀ aryl or 5- to 10-membered heteroaryl, wherein the C₆-C₁₀ aryl or 5- to 10-membered heteroaryl is optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl;
R₂ is C₁-C₆ alkyl, C₂-C₆ alkenyl, C₂-C₆ alkynyl, C₃-C₁₀ cycloalkyl, or 3- to 10-membered heterocyclyl;
R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ;
R₃ₐ is halogen, —OH, —O(C₁-C₆ alkyl), —NH₂, —NH(C₁-C₆ alkyl), —N(C₁-C₆ alkyl)₂, —N(C₁-C₆ alkyl)-SO₂—(C₁-C₆ alkyl), —NHC(O)—(C₁-C₆ alkyl), —NHC(O)—NH₂, —NHC(O)—NH(C₁-C₆ alkyl), —NHC(O)—N(C₁-C₆ alkyl)₂, C₃-C₁₀ cycloalkyl, or 3- to 10-membered heterocyclyl;
R₄ is C₁-C₆ alkyl optionally substituted with one or more R₄ₐ;
R₄ₐ is —OH, —O(C₁-C₆ alkyl), —NH₂, —NH(C₁-C₆ alkyl), —N(C₁-C₆ alkyl)₂, or —SO₂—(C₁-C₆ alkyl); and
R₅ is H or halogen.

In some embodiments, the compound is a compound of Formula (I), wherein:
X is CR₃ or N;
Y is S or NR₄;
R₁ is H, —C(O)—R₁ₐ, —C(NH)—R₁ₐ, C₁-C₆ alkyl, or 5- to 10-membered heteroaryl;
R₁ₐ is —O(C₁-C₆ alkyl), —NH₂, —NH(C₁-C₆ alkyl), C₁-C₆ alkyl, or C₁-C₆ haloalkyl, wherein the —O(C₁-C₆ alkyl), —NH(C₁-C₆ alkyl), C₁-C₆ alkyl, or C₁-C₆ haloalkyl is optionally substituted with one or more R_{1b};
R_{1b} is C₆-C₁₀ aryl or 5- to 10-membered heteroaryl, wherein the C₆-C₁₀ aryl or 5- to 10-membered heteroaryl is optionally substituted with one or more halogen;
R₂ is C₁-C₆ alkyl;
R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), C₁-C₆ alkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), C₁-C₆ alkyl, or 3- to 10-membered heterocyclyl is optionally substituted with one or more R₃ₐ;
R₃ₐ is halogen, —OH, —O(C₁-C₆ alkyl), —N(C₁-C₆ alkyl)₂, —N(C₁-C₆ alkyl)-SO₂—(C₁-C₆ alkyl), or 3- to 10-membered heterocyclyl;
R₄ is C₁-C₆ alkyl optionally substituted with one or more R₄ₐ; and
R₄ₐ is —OH, —O(C₁-C₆ alkyl), or —SO₂—(C₁-C₆ alkyl).

In some aspects, the present disclosure provides a compound obtainable by, or obtained by, a method for preparing a compound as described herein.

In some embodiments, the present disclosure provides a pharmaceutical composition comprising a compound described herein and one or more pharmaceutically acceptable carriers or excipients.

In some aspects, the present disclosure provides an intermediate as described herein, being suitable for use in a method for preparing a compound as described herein.

In some aspects, the present disclosure provides a method of modulating of MCT (e.g., MCT1) activity (e.g., in vitro or in vivo), comprising contacting a cell with an effective amount of a compound or a pharmaceutically acceptable prodrug, solvate, or salt thereof of the present disclosure.

In some aspects, the present disclosure provides a method of treating or preventing a disease or disorder disclosed herein in a subject in need thereof, comprising administering to the subject a therapeutically effective amount of a compound or a pharmaceutically acceptable prodrug, solvate, or salt thereof of the present disclosure or a pharmaceutical composition of the present disclosure.

In some aspects, the present disclosure provides a compound or a pharmaceutically acceptable prodrug, solvate, or salt thereof of the present disclosure for use in modulating MCT (e.g., MCT1) activity (e.g., in vitro or in vivo).

In some aspects, the present disclosure provides a compound or a pharmaceutically acceptable prodrug, solvate, or salt thereof of the present disclosure or a pharmaceutically acceptable salt thereof for use in treating or preventing a disease or disorder disclosed herein.

In some aspects, the present disclosure provides use of a compound or a pharmaceutically acceptable prodrug, solvate, or salt thereof of the present disclosure in the manufacture of a medicament for modulating MCT (e.g., MCT1) activity (e.g., in vitro or in vivo).

In some aspects, the present disclosure provides use of a compound or a pharmaceutically acceptable prodrug, solvate, or salt thereof of the present disclosure in the manufacture of a medicament for treating or preventing a disease or disorder disclosed herein.

In some aspects, the present disclosure provides a method of preparing a compound or a pharmaceutically acceptable prodrug, solvate, or salt thereof of the present disclosure.

In some aspects, the present disclosure provides a method of a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The disclosure relates to compounds useful for the specific modulation of MCT-dependent cellular processes. In particular, compounds with improved physicochemical, pharmacological and pharmaceutical properties to existing MCT-modulating compounds are desired.

### Compounds of the Present Disclosure

In some aspects, the present disclosure relates to a compound of Formula (I):

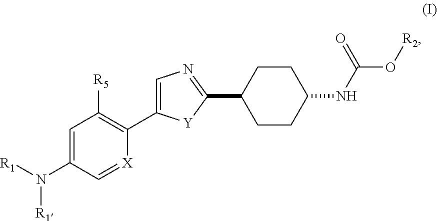

or a pharmaceutically acceptable prodrug, solvate, or salt thereof, wherein:
X is CR₃ or N;
Y is S or NR₄;
R₁ is H, —C(O)—R₁ₐ, —C(NH)—R₁ₐ, C₁-C₆ alkyl, C₂-C₆ alkenyl, C₂-C₆ alkynyl, C₆-C₁₀ aryl, or 5- to 10-membered heteroaryl;
R_{1′} is H or C₁-C₆ alkyl;
R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl, wherein the —O(C₁-C₆ alkyl), —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl is optionally substituted with one or more R_{1b};
R_{1b} is C₆-C₁₀ aryl or 5- to 10-membered heteroaryl, wherein the C₆-C₁₀ aryl or 5- to 10-membered heteroaryl is optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl;
R₂ is C₁-C₆ alkyl, C₂-C₆ alkenyl, C₂-C₆ alkynyl, C₃-C₁₀ cycloalkyl, or 3- to 10-membered heterocyclyl;
R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ;
R₃ₐ is halogen, —OH, —O(C₁-C₆ alkyl), —NH₂, —NH(C₁-C₆ alkyl), —N(C₁-C₆ alkyl)₂, —N(C₁-C₆ alkyl)-SO₂—(C₁-C₆ alkyl), —NHC(O)—(C₁-C₆ alkyl), —NHC(O)—NH₂, —NHC(O)—NH(C₁-C₆ alkyl), —NHC(O)—N(C₁-C₆ alkyl)₂, C₃-C₁₀ cycloalkyl, or 3- to 10-membered heterocyclyl;
R₄ is C₁-C₆ alkyl optionally substituted with one or more R₄ₐ;
R₄ₐ is —OH, —O(C₁-C₆ alkyl), —NH₂, —NH(C₁-C₆ alkyl), —N(C₁-C₆ alkyl)₂, or —SO₂—(C₁-C₆ alkyl); and
R₅ is H or halogen.

It is understood that, for a compound of Formula (I), X, Y, R₁, R_{1′}, R₁ₐ, R_{1b}, R₂, R₃, R₃ₐ, R₄, R₄ₐ, and R₅ can each be, where applicable, selected from the groups described herein, and any group described herein for any of X, Y, R₁, R_{1′}, R₁ₐ, R_{1b}, R₂, R₃, R₃ₐ, R₄, R₄ₐ, and R₅ can be combined, where applicable, with any group described herein for one or more of the remainder of X, Y, R₁, R_{1′}, R₁ₐ, R_{1b}, R₂, R₃, R₃ₐ, R₄, R₄ₐ, and R₅.

In some embodiments, X is CR₃ or N.

In some embodiments, X is CR₃.

In some embodiments, X is N.

In some embodiments, Y is S or NR₄.

In some embodiments, Y is S.

In some embodiments, Y is NR₄.

In some embodiments, R₁ is H, —C(O)—R₁ₐ, —C(NH)—R₁ₐ, C₁-C₆ alkyl, C₂-C₆ alkenyl, C₂-C₆ alkynyl, C₆-C₁₀ aryl, or 5- to 10-membered heteroaryl.

In some embodiments, R₁ is H.

In some embodiments, R₁ is —C(O)—R₁ₐ, —C(NH)—R₁ₐ, C₁-C₆ alkyl, C₂-C₆ alkenyl, C₂-C₆ alkynyl, C₆-C₁₀ aryl, or 5- to 10-membered heteroaryl.

In some embodiments, R₁ is —C(O)—R₁ₐ or —C(NH)—R₁ₐ.

In some embodiments, R₁ is —C(O)—R₁ₐ.

In some embodiments, R₁ is —C(NH)—R₁ₐ.

In some embodiments, R₁ is C₁-C₆ alkyl, C₂-C₆ alkenyl, or C₂-C₆ alkynyl.

In some embodiments, R₁ is C₁-C₆ alkyl.

In some embodiments, R₁ is methyl. In some embodiments, R₁ is ethyl. In some embodiments, R₁ is propyl. In some embodiments, R₁ is butyl. In some embodiments, R₁ is pentyl. In some embodiments, R₁ is hexyl. In some embodiments, R₁ is isopropyl. In some embodiments, R₁ is isobutyl. In some embodiments, R₁ is isopentyl. In some embodiments, R₁ is isohexyl. In some embodiments, R₁ is secbutyl. In some embodiments, R₁ is secpentyl. In some embodiments, R₁ is sechexyl. In some embodiments, R₁ is tertbutyl.

In some embodiments, R₁ is C₂-C₆ alkenyl.

In some embodiments, R₁ is C₂ alkenyl. In some embodiments, R₁ is C₃ alkenyl. In some embodiments, R₁ is C₄ alkenyl. In some embodiments, R₁ is C₅ alkenyl. In some embodiments, R₁ is C₆ alkenyl.

In some embodiments, R₁ is C₂-C₆ alkynyl.

In some embodiments, R₁ is C₂ alkynyl. In some embodiments, R₁ is C₃ alkynyl. In some embodiments, R₁ is C₄ alkynyl. In some embodiments, R₁ is C₅ alkynyl. In some embodiments, R₁ is C₆ alkynyl.

In some embodiments, R₁ is C₆-C₁₀ aryl or 5- to 10-membered heteroaryl.

In some embodiments, R₁ is C₆-C₁₀ aryl.

In some embodiments, R₁ is C₆ aryl (e.g., phenyl).

In some embodiments, R₁ is C₈ aryl. In some embodiments, R₁ is C₁₀ aryl.

In some embodiments, R₁ is 5- to 10-membered heteroaryl.

In some embodiments, R₁ is 5-membered heteroaryl. In some embodiments, R₁ is 6-membered heteroaryl. In some embodiments, R₁ is 7-membered heteroaryl. In some embodiments, R₁ is 8-membered heteroaryl. In some embodiments, R₁ is 9-membered heteroaryl. In some embodiments, R₁ is 10-membered heteroaryl.

In some embodiments, R_{1′} is H or C₁-C₆ alkyl.

In some embodiments, R_{1′} is H.

In some embodiments, R_{1′} is C₁-C₆ alkyl.

In some embodiments, R_{1′} is methyl. In some embodiments, R_{1′} is ethyl. In some embodiments, R_{1′} is propyl. In some embodiments, R_{1′} is butyl. In some embodiments, R_{1′} is pentyl. In some embodiments, R_{1′} is hexyl. In some embodiments, R_{1′} is isopropyl. In some embodiments, R_{1′} is isobutyl. In some embodiments, R_{1′} is isopentyl. In some embodiments, R_{1′} is isohexyl. In some embodiments, R_{1′} is secbutyl. In some embodiments, R_{1′} is secpentyl. In some embodiments, R_{1′} is sechexyl. In some embodiments, R_{1′} is tertbutyl.

In some embodiments, R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl.

In some embodiments, R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl, wherein the —O(C₁-C₆ alkyl), —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl is optionally substituted with one or more R_{1b}.

In some embodiments, R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl, wherein the —O(C₁-C₆ alkyl), —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl is substituted with one or more R_{1b}.

In some embodiments, R₁ₐ is —OH or —O(C₁-C₆ alkyl).

In some embodiments, R₁ₐ is —OH.

In some embodiments, R₁ₐ is —O(C₁-C₆ alkyl).

In some embodiments, R₁ₐ is —O(C₁-C₆ alkyl) optionally substituted with one or more R_{1b}.

In some embodiments, R₁ₐ is —O(C₁-C₆ alkyl) substituted with one or more R_{1b}.

In some embodiments, R₁ₐ is —NH₂, —NH(C₁-C₆ alkyl), or —N(C₁-C₆ alkyl)₂.

In some embodiments, R₁ₐ is —NH₂.

In some embodiments, R₁ₐ is —NH(C₁-C₆ alkyl).

In some embodiments, R₁ₐ is —NH(C₁-C₆ alkyl) optionally substituted with one or more R_{1b}.

In some embodiments, R₁ₐ is —NH(C₁-C₆ alkyl) substituted with one or more R_{1b}.

In some embodiments, R₁ₐ is —N(C₁-C₆ alkyl)₂.

In some embodiments, R₁ₐ is —N(C₁-C₆ alkyl)₂ optionally substituted with one or more R_{1b}.

In some embodiments, R₁ₐ is —N(C₁-C₆ alkyl)₂ substituted with one or more R_{1b}.

In some embodiments, R₁ₐ is C₁-C₆ alkyl, C₂-C₆ alkenyl, C₂-C₆ alkynyl, or C₁-C₆ haloalkyl.

In some embodiments, R₁ₐ is C₁-C₆ alkyl.

In some embodiments, R₁ₐ is C₁-C₆ alkyl optionally substituted with one or more R_{1b}.

In some embodiments, R₁ₐ is C₁-C₆ alkyl substituted with one or more R_{1b}.

In some embodiments, R₁ₐ is methyl. In some embodiments, R₁ₐ is ethyl. In some embodiments, R₁ₐ is propyl. In some embodiments, R₁ₐ is butyl. In some embodiments, R₁ₐ is pentyl. In some embodiments, R₁ₐ is hexyl. In some embodiments, R₁ₐ is isopropyl. In some embodiments, R₁ₐ is isobutyl. In some embodiments, R₁ₐ is isopentyl. In some embodiments, R₁ₐ is isohexyl. In some embodiments, R₁ₐ is secbutyl. In some embodiments, R₁ₐ is secpentyl. In some embodiments, R₁ₐ is sechexyl. In some embodiments, R₁ₐ is tertbutyl.

In some embodiments, R₁ₐ is methyl optionally substituted with one or more R_{1b}. In some embodiments, R₁ₐ is ethyl optionally substituted with one or more R_{1b}. In some embodiments, R₁ₐ is propyl optionally substituted with one or more R_{1b}. In some embodiments, R₁ₐ is butyl optionally substituted with one or more R_{1b}. In some embodiments, R₁ₐ is pentyl optionally substituted with one or more R_{1b}. In some embodiments, R₁ₐ is hexyl optionally substituted with one or more R_{1b}. In some embodiments, R₁ₐ is isopropyl optionally substituted with one or more R_{1b}. In some embodiments, R₁ₐ is isobutyl optionally substituted with one or more R_{1b}. In some embodiments, R₁ₐ is isopentyl optionally substituted with one or more R_{1b}. In some embodiments, R₁ₐ is isohexyl optionally substituted with one or more R_{1b}. In some embodiments, R₁ₐ is secbutyl optionally substituted with one or more R_{1b}. In some embodiments, R₁ₐ is secpentyl optionally substituted with one or more R_{1b}. In some embodiments, R₁ₐ is sechexyl optionally substituted with one or more R_{1b}. In some embodiments, R₁ₐ is tertbutyl optionally substituted with one or more R_{1b}.

In some embodiments, R₁ₐ is methyl substituted with one or more R_{1b}. In some embodiments, R₁ₐ is ethyl substituted with one or more R_{1b}. In some embodiments, R₁ₐ is propyl substituted with one or more R_{1b}. In some embodiments, R₁ₐ is butyl substituted with one or more R_{1b}. In some embodiments, R₁ₐ is pentyl substituted with one or more R_{1b}. In some embodiments, R₁ₐ is hexyl substituted with one or more R_{1b}. In some embodiments, R₁ₐ is isopropyl substituted with one or more R_{1b}. In some embodiments, R₁ₐ is isobutyl substituted with one or more R_{1b}. In some embodiments, R₁ₐ is isopentyl substituted with one or more R_{1b}. In some embodiments, R₁ₐ is isohexyl substituted with one or more R_{1b}. In some embodiments, R₁ₐ is secbutyl substituted with one or more R_{1b}. In some embodiments, R₁ₐ is secpentyl substituted with one or more R_{1b}. In some embodiments, R₁ₐ is sechexyl substituted with one or more R_{1b}. In some embodiments, R₁ₐ is tertbutyl substituted with one or more R_{1b}.

In some embodiments, R₁ₐ is C₂-C₆ alkenyl.

In some embodiments, R₁ₐ is C₂-C₆ alkenyl optionally substituted with one or more R_{1b}.

In some embodiments, R₁ₐ is C₂-C₆ alkenyl substituted with one or more R_{1b}.

In some embodiments, R₁ₐ is C₂-C₆ alkynyl.

In some embodiments, R₁ₐ is C₂-C₆ alkynyl optionally substituted with one or more R_{1b}.

In some embodiments, R₁ₐ is C₂-C₆ alkynyl substituted with one or more R_{1b}.

In some embodiments, R₁ₐ is C₁-C₆ haloalkyl.

In some embodiments, R₁ₐ is C₁-C₆ haloalkyl optionally substituted with one or more R_{1b}.

In some embodiments, R₁ₐ is C₁-C₆ haloalkyl substituted with one or more R_{1b}.

In some embodiments, R_{1b} is C₆-C₁₀ aryl or 5- to 10-membered heteroaryl.

In some embodiments, R_{1b} is C₆-C₁₀ aryl or 5- to 10-membered heteroaryl, wherein the is C₆-C₁₀ aryl or 5- to 10-membered heteroaryl is optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is C₆-C₁₀ aryl or 5- to 10-membered heteroaryl, wherein the C₆-C₁₀ aryl or 5- to 10-membered heteroaryl is substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is C₆-C₁₀ aryl.

In some embodiments, R_{1b} is C₆-C₁₀ aryl optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is C₆-C₁₀ aryl substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is C₆ aryl.

In some embodiments, R_{1b} is phenyl.

In some embodiments, R_{1b} is C₆ aryl optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is C₆ aryl substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is 5- to 10-membered heteroaryl.

In some embodiments, R_{1b} is 5- to 10-membered heteroaryl, wherein the 5- to 10-membered heteroaryl is optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R_{1b} is 5- to 10-membered heteroaryl, wherein the 5- to 10-membered heteroaryl is substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl.

In some embodiments, R₂ is C₁-C₆ alkyl, C₂-C₆ alkenyl, C₂-C₆ alkynyl, C₃-C₁₀ cycloalkyl, or 3- to 10-membered heterocyclyl.

In some embodiments, R₂ is C₁-C₆ alkyl, C₂-C₆ alkenyl, or C₂-C₆ alkynyl.

In some embodiments, R₂ is C₁-C₆ alkyl.

In some embodiments, R₂ is methyl. In some embodiments, R₂ is ethyl. In some embodiments, R₂ is propyl. In some embodiments, R₂ is butyl. In some embodiments, R₂ is pentyl. In some embodiments, R₂ is hexyl. In some embodiments, R₂ is isopropyl. In some embodiments, R₂ is isobutyl. In some embodiments, R₂ is isopentyl. In some embodiments, R₂ is isohexyl. In some embodiments, R₂ is secbutyl. In some embodiments, R₂ is secpentyl. In some embodiments, R₂ is sechexyl. In some embodiments, R₂ is tertbutyl.

In some embodiments, R₂ is C₂-C₆ alkenyl.

In some embodiments, R₂ is C₂-C₆ alkynyl.

In some embodiments, R₂ is C₃-C₁₀ cycloalkyl or 3- to 10-membered heterocyclyl.

In some embodiments, R₂ is C₃-C₁₀ cycloalkyl.

In some embodiments, R₂ is C₃ cycloalkyl. In some embodiments, R₂ is C₄ cycloalkyl. In some embodiments, R₂ is C₅ cycloalkyl. In some embodiments, R₂ is C₆ cycloalkyl. In some embodiments, R₂ is C₇ cycloalkyl. In some embodiments, R₂ is C₈ cycloalkyl. In some embodiments, R₂ is C₉ cycloalkyl. In some embodiments, R₂ is C₁₀ cycloalkyl.

In some embodiments, R₂ is 3- to 10-membered heterocyclyl.

In some embodiments, R₂ is 3-membered heterocyclyl. In some embodiments, R₂ is 4-membered heterocyclyl. In some embodiments, R₂ is 5-membered heterocyclyl. In some embodiments, R₂ is 6-membered heterocyclyl. In some embodiments, R₂ is 7-membered heterocyclyl. In some embodiments, R₂ is 8-membered heterocyclyl. In some embodiments, R₂ is 9-membered heterocyclyl. In some embodiments, R₂ is 10-membered heterocyclyl.

In some embodiments, R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl.

In some embodiments, R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ.

In some embodiments, R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is substituted with one or more R₃ₐ.

In some embodiments, R₃ is H.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is substituted with one or more R₃ₐ.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), or —NHC(O)—N(C₁-C₆ alkyl)₂ is optionally substituted with one or more R₃ₐ.

In some embodiments, R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), or —NHC(O)—N(C₁-C₆ alkyl)₂ is substituted with one or more R₃ₐ.

In some embodiments, R₃ is halogen or —CN.

In some embodiments, R₃ is —OH or —O(C₁-C₆ alkyl).

In some embodiments, R₃ is —OH or —O(C₁-C₆ alkyl), wherein the —O(C₁-C₆ alkyl) is optionally substituted with one or more R₃ₐ.

In some embodiments, R₃ is —OH or —O(C₁-C₆ alkyl), wherein the —O(C₁-C₆ alkyl) is substituted with one or more R₃ₐ.

In some embodiments, R₃ is —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂.

In some embodiments, R₃ is —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂, wherein the —NH(C₁-C₆ alkyl), —N(C₁-C₆ alkyl)₂, —NHC(O)—(C₁-C₆ alkyl), —NHC(O)—NH(C₁-C₆ alkyl), or —NHC(O)—N(C₁-C₆ alkyl)₂, is optionally substituted with one or more R₃ₐ.

In some embodiments, R₃ is —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), or —NHC(O)—N(C₁-C₆ alkyl)₂, wherein the —NH(C₁-C₆ alkyl), —N(C₁-C₆ alkyl)₂, —NHC(O)—(C₁-C₆ alkyl), —NHC(O)—NH(C₁-C₆ alkyl), or —NHC(O)—N(C₁-C₆ alkyl)₂, is substituted with one or more R₃ₐ.

In some embodiments, R₃ is —OH.

In some embodiments, R₃ is —OCH₃.

In some embodiments, R₃ is —F.

In some embodiments, R₃ is —NH₂.

In some embodiments, R₃ is —NH(C₁-C₆ alkyl).

In some embodiments, R₃ is —NH(C₁-C₆ alkyl) is optionally substituted with one or more R₃ₐ.

In some embodiments, R₃ is —NH(C₁-C₆ alkyl) substituted with one or more R₃ₐ.

In some embodiments, R₃ is —N(C₁-C₆ alkyl)₂.

In some embodiments, R₃ is —N(C₁-C₆ alkyl)₂ optionally substituted with one or more R₃ₐ.

In some embodiments, R₃ is —N(C₁-C₆ alkyl)₂ substituted with one or more R₃ₐ.

In some embodiments, R₃ is —NHC(O)—(C₁-C₆ alkyl).

In some embodiments, R₃ is —NHC(O)—(C₁-C₆ alkyl) optionally substituted with one or more R₃ₐ.

In some embodiments, R₃ is —NHC(O)—(C₁-C₆ alkyl) substituted with one or more R₃ₐ.

In some embodiments, R₃ is —NHC(O)—NH₂.

In some embodiments, R₃ is —NHC(O)—NH₂ optionally substituted with one or more R₃ₐ.

In some embodiments, R₃ is —NHC(O)—NH₂ substituted with one or more R₃ₐ.

In some embodiments, R₃ is —NHC(O)—NH(C₁-C₆ alkyl).

In some embodiments, R₃ is —NHC(O)—NH(C₁-C₆ alkyl) optionally substituted with one or more R₃ₐ.

In some embodiments, R₃ is —NHC(O)—NH(C₁-C₆ alkyl) substituted with one or more R₃ₐ.

In some embodiments, R₃ is —NHC(O)—N(C₁-C₆ alkyl)₂.

In some embodiments, R₃ is —NHC(O)—N(C₁-C₆ alkyl)₂ optionally substituted with one or more R₃ₐ.

In some embodiments, R₃ is —NHC(O)—N(C₁-C₆ alkyl)₂ substituted with one or more R₃ₐ.

In some embodiments, R₃ is C₁-C₆ alkyl, C₂-C₆ alkenyl, C₂-C₆ alkynyl, C₃-C₁₀ cycloalkyl, or 3- to 10-membered heterocyclyl.

In some embodiments, R₃ is C₁-C₆ alkyl, C₂-C₆ alkenyl, C₂-C₆ alkynyl, C₃-C₁₀ cycloalkyl, or 3- to 10-membered heterocyclyl, wherein the C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ.

In some embodiments, R₃ is C₁-C₆ alkyl, C₂-C₆ alkenyl, C₂-C₆ alkynyl, C₃-C₁₀ cycloalkyl, or 3- to 10-membered heterocyclyl, wherein the C₁-C₆ alkyl, C₂-C₆ alkenyl, C₂-C₆ alkynyl, C₃-C₁₀ cycloalkyl, or 3- to 10-membered heterocyclyl is substituted with one or more R₃ₐ.

In some embodiments, R₃ is C₁-C₆ alkyl.

In some embodiments, R₃ is C₁-C₆ alkyl optionally substituted with one or more R₃ₐ.

In some embodiments, R₃ is C₁-C₆ alkyl substituted with one or more R₃ₐ.

In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₃ is sechexyl. In some embodiments, R₃ is tertbutyl.

In some embodiments, R₃ is C₂-C₆ alkenyl.

In some embodiments, R₃ is C₂-C₆ alkenyl optionally substituted with one or more R₃ₐ.

In some embodiments, R₃ is C₂-C₆ alkenyl substituted with one or more R₃ₐ.

In some embodiments, R₃ is C₂-C₆ alkynyl.

In some embodiments, R₃ is C₂-C₆ alkynyl optionally substituted with one or more R₃ₐ.

In some embodiments, R₃ is C₂-C₆ alkynyl substituted with one or more R₃ₐ.

In some embodiments, R₃ is C₃-C₁₀ cycloalkyl.

In some embodiments, R₃ is C₃-C₁₀ cycloalkyl optionally substituted with one or more R₃ₐ.

In some embodiments, R₃ is C₃-C₁₀ cycloalkyl substituted with one or more R₃ₐ.

In some embodiments, R₃ is 3- to 10-membered heterocyclyl.

In some embodiments, R₃ is 3- to 10-membered heterocyclyl optionally substituted with one or more R₃ₐ.

In some embodiments, R₃ is 3- to 10-membered heterocyclyl substituted with one or more R₃ₐ.

In some embodiments, R₃ₐ is halogen, —OH, —O(C₁-C₆ alkyl), —NH₂, —NH(C₁-C₆ alkyl), —N(C₁-C₆ alkyl)₂, —N(C₁-C₆ alkyl)-SO₂—(C₁-C₆ alkyl), —NHC(O)—(C₁-C₆ alkyl), —NHC(O)—NH₂, —NHC(O)—NH(C₁-C₆ alkyl), —NHC(O)—N(C₁-C₆ alkyl)₂, C₃-C₁₀ cycloalkyl, or 3- to 10-membered heterocyclyl.

In some embodiments, R₃ₐ is halogen.

In some embodiments, R₃ₐ is —OH or —O(C₁-C₆ alkyl).

In some embodiments, R₃ₐ is —OH.

In some embodiments, R₃ₐ is —O(C₁-C₆ alkyl).

In some embodiments, R₃ₐ is —NH₂, —NH(C₁-C₆ alkyl), —N(C₁-C₆ alkyl)₂, —N(C₁-C₆ alkyl)-SO₂—(C₁-C₆ alkyl), —NHC(O)—(C₁-C₆ alkyl), —NHC(O)—NH₂, —NHC(O)—NH(C₁-C₆ alkyl), or —NHC(O)—N(C₁-C₆ alkyl)₂.

In some embodiments, R₃ₐ is —NH₂.

In some embodiments, R₃ₐ is —NH(C₁-C₆ alkyl).

In some embodiments, R₃ₐ is —N(C₁-C₆ alkyl)₂.

In some embodiments, R₃ₐ is —N(C₁-C₆ alkyl)-SO₂—(C₁-C₆ alkyl).

In some embodiments, R₃ₐ is —NHC(O)—(C₁-C₆ alkyl).

In some embodiments, R₃ₐ is —NHC(O)—NH₂.

In some embodiments, R₃ₐ is —NHC(O)—NH(C₁-C₆ alkyl).

In some embodiments, R₃ₐ is —NHC(O)—N(C₁-C₆ alkyl)₂.

In some embodiments, R₃ₐ is C₃-C₁₀ cycloalkyl or 3- to 10-membered heterocyclyl.

In some embodiments, R₃ₐ is C₃-C₁₀ cycloalkyl.

In some embodiments, R₃ₐ is 3- to 10-membered heterocyclyl.

In some embodiments, R₄ is C₁-C₆ alkyl.

In some embodiments, R₄ is C₁-C₆ alkyl optionally substituted with one or more R₄ₐ.

In some embodiments, R₄ is C₁-C₆ alkyl substituted with one or more R₄ₐ.

In some embodiments, R₄ is methyl. In some embodiments, R₄ is ethyl. In some embodiments, R₄ is propyl. In some embodiments, R₄ is butyl. In some embodiments, R₄ is pentyl. In some embodiments, R₄ is hexyl. In some embodiments, R₄ is isopropyl. In some embodiments, R₄ is isobutyl. In some embodiments, R₄ is isopentyl. In some embodiments, R₄ is isohexyl. In some embodiments, R₄ is secbutyl. In some embodiments, R₄ is secpentyl. In some embodiments, R₄ is sechexyl. In some embodiments, R₄ is tertbutyl.

In some embodiments, R₄ is methyl optionally substituted with one or more R₄ₐ. In some embodiments, R₄ is ethyl optionally substituted with one or more R₄ₐ. In some embodiments, R₄ is propyl optionally substituted with one or more R₄ₐ. In some embodiments, R₄ is butyl optionally substituted with one or more R₄ₐ. In some embodiments, R₄ is pentyl optionally substituted with one or more R₄ₐ. In some embodiments, R₄ is hexyl optionally substituted with one or more R₄ₐ. In some embodiments, R₄ is isopropyl optionally substituted with one or more R₄ₐ. In some embodiments, R₄ is isobutyl optionally substituted with one or more R₄ₐ. In some embodiments, R₄ is isopentyl optionally substituted with one or more R₄ₐ. In some embodiments, R₄ is isohexyl optionally substituted with one or more R₄ₐ. In some embodiments, R₄ is secbutyl optionally substituted with one or more R₄ₐ. In some embodiments, R₄ is secpentyl optionally substituted with one or more R₄ₐ. In some embodiments, R₄ is sechexyl optionally substituted with one or more R₄ₐ. In some embodiments, R₄ is tertbutyl optionally substituted with one or more R₄ₐ.

In some embodiments, R₄ is methyl substituted with one or more R₄ₐ. In some embodiments, R₄ is ethyl substituted with one or more R₄ₐ. In some embodiments, R₄ is propyl substituted with one or more R₄ₐ. In some embodiments, R₄ is butyl substituted with one or more R₄ₐ. In some embodiments, R₄ is pentyl substituted with one or more R₄ₐ. In some embodiments, R₄ is hexyl substituted with one or more R₄ₐ. In some embodiments, R₄ is isopropyl substituted with one or more R₄ₐ. In some embodiments, R₄ is isobutyl substituted with one or more R₄ₐ. In some embodiments, R₄ is isopentyl substituted with one or more R₄ₐ. In some embodiments, R₄ is isohexyl substituted with one or more R₄ₐ. In some embodiments, R₄ is secbutyl substituted with one or more R₄ₐ. In some embodiments, R₄ is secpentyl substituted with one or more R₄ₐ. In some embodiments, R₄ is sechexyl substituted with one or more R₄ₐ. In some embodiments, R₄ is tertbutyl substituted with one or more R₄ₐ.

In some embodiments, R₄ₐ is —OH, —O(C₁-C₆ alkyl), —NH₂, —NH(C₁-C₆ alkyl), —N(C₁-C₆ alkyl)₂, or —SO₂—(C₁-C₆ alkyl).

In some embodiments, R₄ₐ is —OH or —O(C₁-C₆ alkyl).

In some embodiments, R₄ₐ is —OH.

In some embodiments, R₄ₐ is —O(C₁-C₆ alkyl).

In some embodiments, R₄ₐ is —NH₂, —NH(C₁-C₆ alkyl), or —N(C₁-C₆ alkyl)₂.

In some embodiments, R₄ₐ is —NH₂.

In some embodiments, R₄ₐ is —NH(C₁-C₆ alkyl).

In some embodiments, R₄ₐ is —N(C₁-C₆ alkyl)₂.

In some embodiments, R₄ₐ is —SO₂—(C₁-C₆ alkyl).

In some embodiments, R₅ is H or halogen.

In some embodiments, R₅ is H.

In some embodiments, R₅ is halogen.

In some embodiments, the compound is of Formula (Ia):

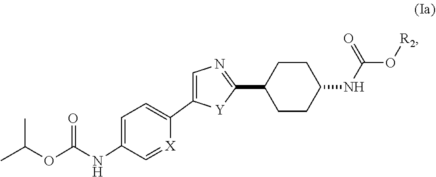

or a pharmaceutically acceptable prodrug, solvate, or salt thereof.

In some embodiments, the compound is of Formula (Ib):

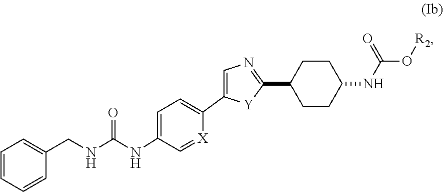

or a pharmaceutically acceptable prodrug, solvate, or salt thereof.

In some embodiments, the compound is of Formula (Ic):

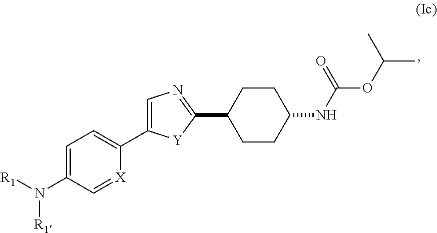

or a pharmaceutically acceptable prodrug, solvate, or salt thereof.

In some embodiments, the compound is of Formula (Id):

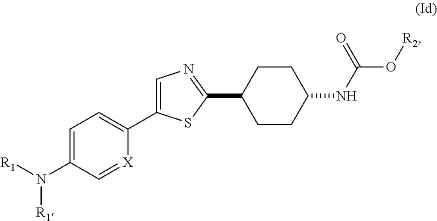

or a pharmaceutically acceptable prodrug, solvate, or salt thereof.

In some embodiments, the compound is of Formula (Ie):

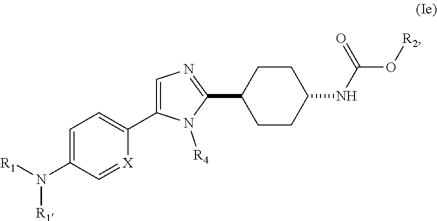

or a pharmaceutically acceptable prodrug, solvate, or salt thereof.

In some embodiments, the compound is of Formula (If):

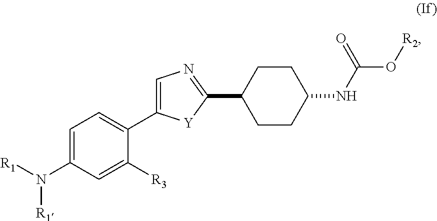

or a pharmaceutically acceptable prodrug, solvate, or salt thereof.

In some embodiments, the compound is of Formula (Ig):

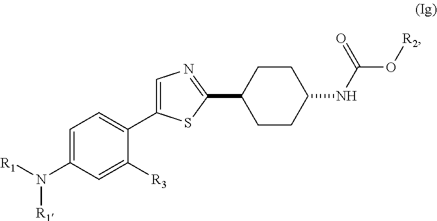

or a pharmaceutically acceptable prodrug, solvate, or salt thereof.

In some embodiments, the compound is selected from the compounds described in Table 1 and pharmaceutically acceptable prodrugs, solvates, or salts thereof.

In some embodiments, the compound is selected from the compounds described in Table 1 and pharmaceutically acceptable prodrugs, solvates, or salts thereof.

In some embodiments, the compound is selected from the compounds described in Table 1.

TABLE 1

| Ex. | | |
|---|---|---|
| # | Structure | Name |
| 1 | 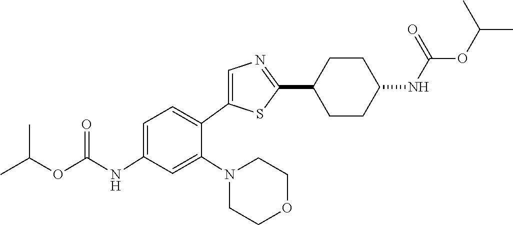 | isopropyl ((trans)-4-(5-(4- ((isopropoxycarbonyl)amino)-2- morpholinophenyl)thiazol-2- yl)cyclohexyl)carbamate |
| 2 | 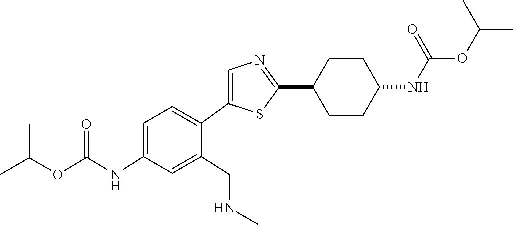 | isopropyl ((trans)-4-(5-(4- ((isopropoxycarbonyl)amino)-2- ((methylamino)methyl)phenyl) thiazol-2-yl)cyclohexyl)carbamate |
| 3 | 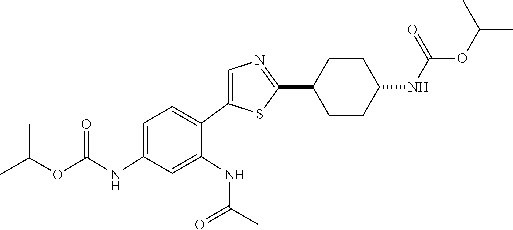 | isopropyl ((tran)-4-(5-(2- acetamido-4- ((isopropoxycarbonyl)amino) phenyl)thiazol-2- yl)cyclohexyl)carbamate |
| 4 | 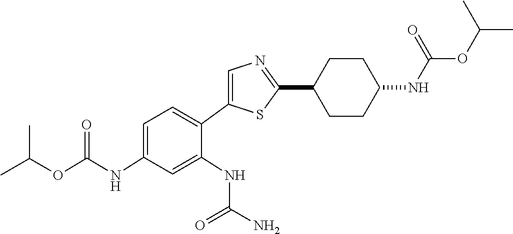 | isopropyl ((trans)-4-(5-(4- ((isopropoxycarbonyl)amino)-2- ureidophenyl)thiazol-2- yl)cyclohexyl)carbamate |
| 5 | 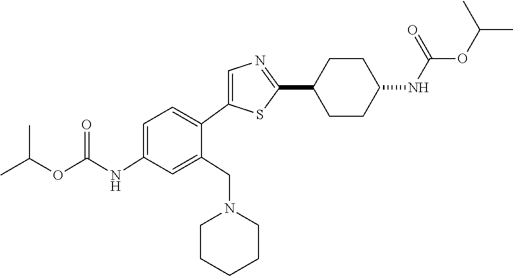 | isopropyl ((trans)-4-(5-(4- ((isopropoxycarbonyl)amino)-2- (piperidin-1- ylmethyl)phenyl)thiazol-2- yl)cyclohexyl)carbamate |
| 6 | 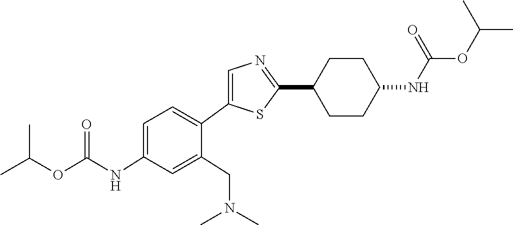 | isopropyl ((trans)-4-(5-(2- ((dimethylamino)methyl)-4- ((isopropoxycarbonyl)amino) phenyl)thiazol-2- yl)cyclohexyl)carbamate |
| 7 | 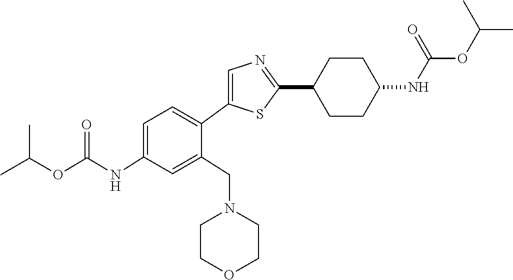 | isopropyl ((trans)-4-(5-(4- ((isopropoxycarbonyl)amino)-2- (morpholinomethyl)phenyl)thiazol- 2-yl)cyclohexyl)carbamate |
| 8 | 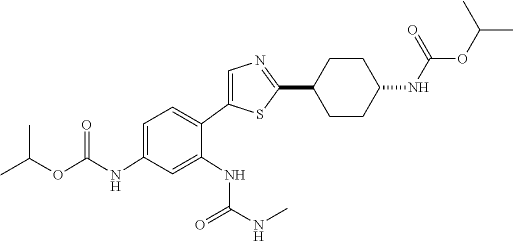 | isopropyl ((trans)-4-(5-(4- ((isopropoxycarbonyl)amino)-2- (3-methylureido)phenyl)thiazol- 2-yl)cyclohexyl)carbamate |
| 9 | 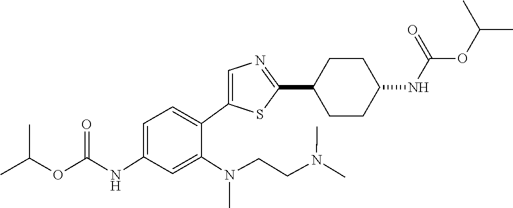 | isopropyl ((trans)-4-(5-(2-((2- (dimethylamino)ethyl)(methyl)amino)-4- ((isopropoxycarbonyl)amino)phenyl) thiazol-2-yl)cyclohexyl)carbamate |
| 10 | 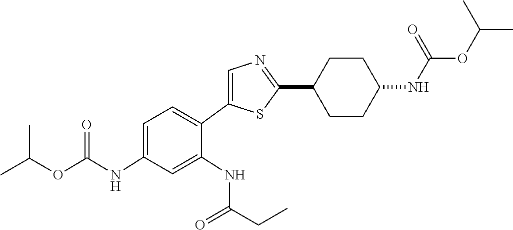 | isopropyl ((trans)-4-(5-(4- ((isopropoxycarbonyl)amino)-2- propionamidophenyl)thiazol-2- yl)cyclohexyl)carbamate |
| 11 | 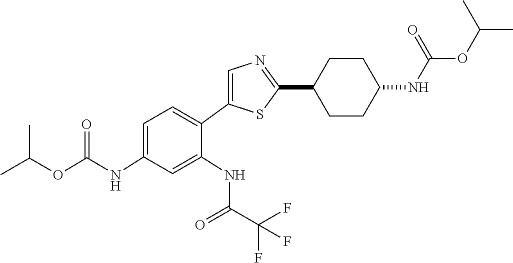 | isopropyl ((trans)-4-(5-(4- ((isopropoxycarbonyl)amino)-2- (2,2,2-trifluoroacetamido)phenyl)thiazol- 2-yl)cyclohexyl)carbamate |
| 12 | 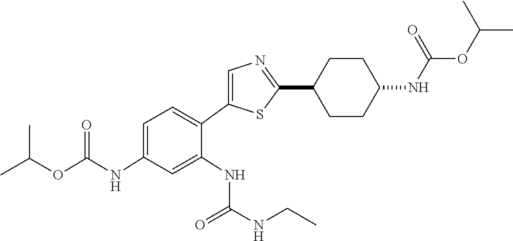 | isopropyl ((trans)-4-(5-(2-(3- ethylureido)-4- ((isopropoxycarbonyl)amino)phenyl) thiazol-2-yl)cyclohexyl)carbamate |
| 13 | 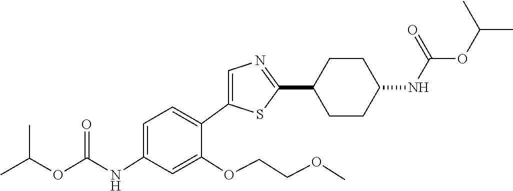 | isopropyl ((trans)-4-(5-(4- ((isopropoxycarbonyl)amino)-2-(2- methoxyethoxy)phenyl)thiazol-2- yl)cyclohexyl)carbamate |
| 14 | 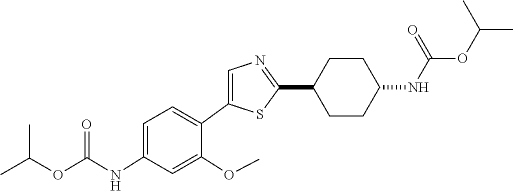 | isopropyl ((trans)-4-(5-(4- ((isopropoxycarbonyl)amino)-2- methoxyphenyl)thiazol-2- yl)cyclohexyl)carbamate |
| 15 | 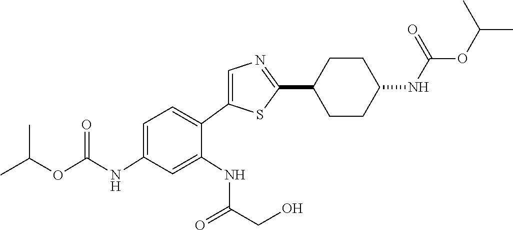 | isopropyl ((trans)-4-(5-(2-(2- hydroxyacetamido)-4- ((isopropoxycarbonyl)amino) phenyl)thiazol-2- yl)cyclohexyl)carbamate |
| 16 | 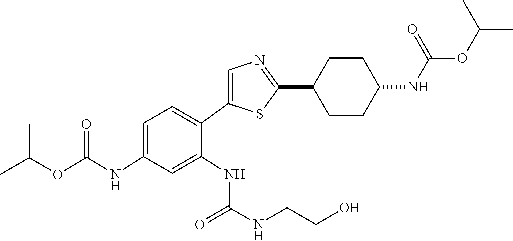 | isopropyl ((trans)-4-(5-(2-(3-(2- hydroxyethyl)ureido)-4- ((isopropoxycarbonyl)amino) phenyl)thiazol-2- yl)cyclohexyl)carbamate |
| 17 | 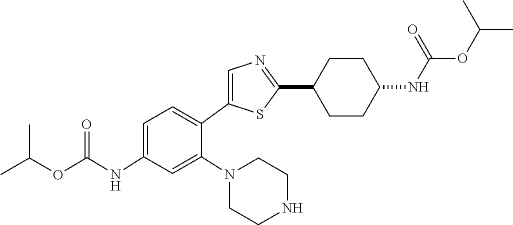 | isopropyl ((trans)-4-(5-(4- ((isopropoxycarbonyl)amino)-2- (piperazin-1-yl)phenyl)thiazol-2- yl)cyclohexyl)carbamate |
| 18 | 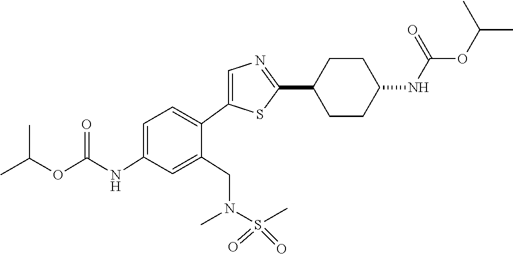 | isopropyl ((trans)-4-(5-(4- ((isopropoxycarbonyl)amino)-2- ((N- methylmethylsulfonamido)methyl) phenyl)thiazol-2- yl)cyclohexyl)carbamate |
| 19 | 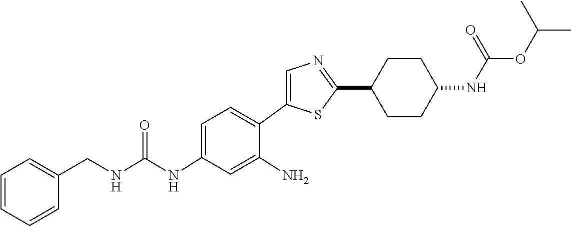 | isopropyl ((trans)-4-(5-(2-amino- 4-(3- benzylureido)phenyl)thiazol-2- yl)cyclohexyl)carbamate |
| 20 | 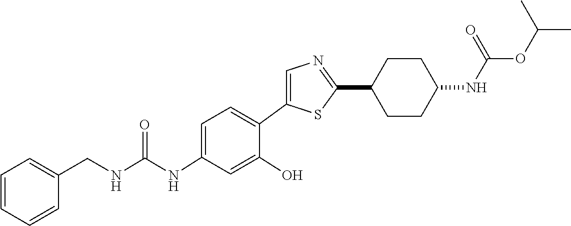 | isopropyl ((trans)-4-(5-(4-(3- benzylureido)-2- hydroxyphenyl)thiazol-2- yl)cyclohexyl)carbamate |
| 21 | 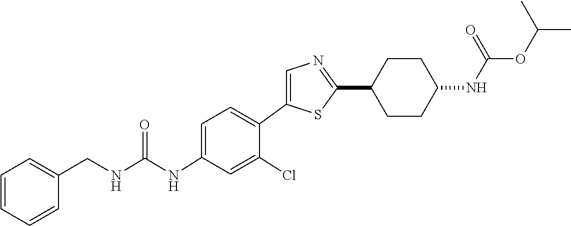 | isopropyl ((trans)-4-(5-(4-(3- benzylureido)-2- chlorophenyl)thiazol-2- yl)cyclohexyl)carbamate |
| 22 | 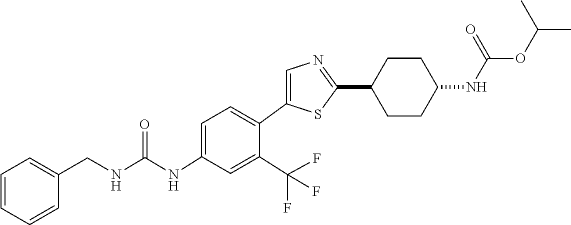 | isopropyl ((trans)-4-(5-(4-(3- benzylureido)-2- (trifluoromethyl)phenyl)thiazol- 2-yl)cyclohexyl)carbamate |
| 23 | 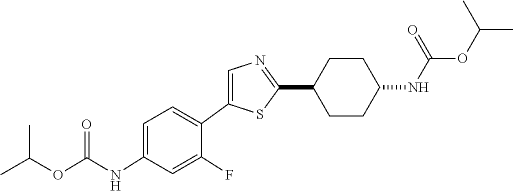 | isopropyl ((trans)-4-(5-(2-fluoro- 4- ((isopropoxycarbonyl)amino) phenyl)thiazol-2- yl)cyclohexyl)carbamate |
| 24 | 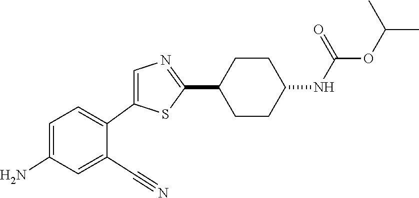 | isopropyl ((trans)-4-(5-(4-amino- 2-cyanophenyl)thiazol-2- yl)cyclohexyl)carbamate |
| 25 | 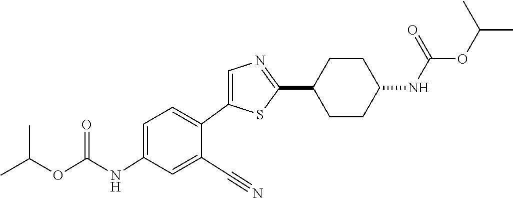 | isopropyl ((trans)-4-(5-(2-cyano- 4- ((isopropoxycarbonyl)amino)phenyl) thiazol-2-yl)cyclohexyl)carbamate |
| 26 | 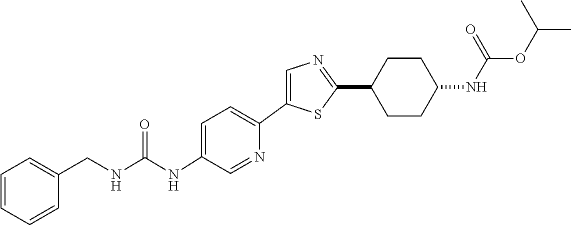 | isopropyl ((trans)-4-(5-(5-(3- benzylureido)pyridin-2- yl)thiazol-2- yl)cyclohexyl)carbamate)carbamate |
| 27 | 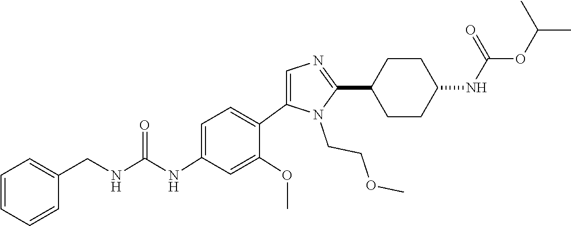 | isopropyl ((trans)-4-(5-(4-(3- benzylureido)-2-methoxyphenyl)- 1-(2-methoxyethyl)-1H-imidazol- 2-yl)cyclohexyl)carbamate |
| 28 | 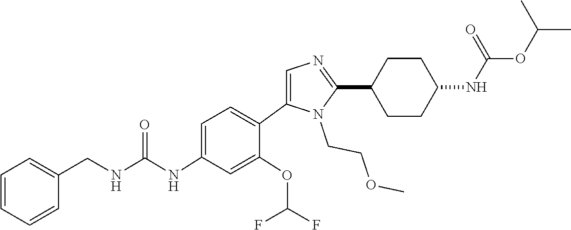 | isopropyl ((trans)-4-(5-(4-(3- benzylureido)-2- (difluoromethoxy)phenyl)-1-(2- methoxyethyl)-1H-imidazol-2- yl)cyclohexyl)carbamate |
| 29 | 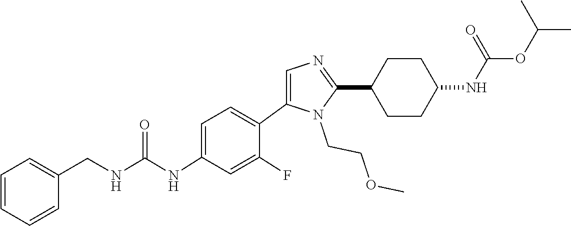 | isopropyl ((trans)-4-(5-(4-(3- benzylureido)-2-fluorophenyl)-1- (2-methoxyethyl)-1H-imidazol-2- yl)cyclohexyl)carbamate |
| 30 | 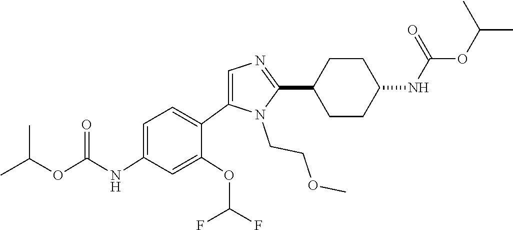 | isopropyl ((trans)-4-(5-(2- (difluoromethoxy)-4- ((isopropoxycarbonyl)amino) phenyl)-1-(2-methoxyethyl)-1H- imidazol-2- yl)cyclohexyl)carbamate |
| 31 | 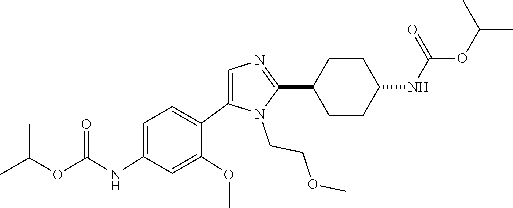 | isopropyl ((trans)-4-(5-(4- ((isopropoxycarbonyl)amino)-2- methoxyphenyl)-1-(2- methoxyethyl)-1H-imidazol-2- yl)cyclohexyl)carbamate |
| 32 | 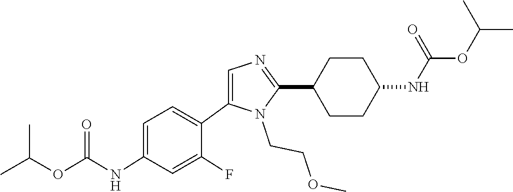 | isopropyl ((trans)-4-(5-(2-fluoro- 4- ((isopropoxycarbonyl)amino) phenyl)-1-(2-methoxyethyl)-1H- imidazol-2- yl)cyclohexyl)carbamate |
| 33 | 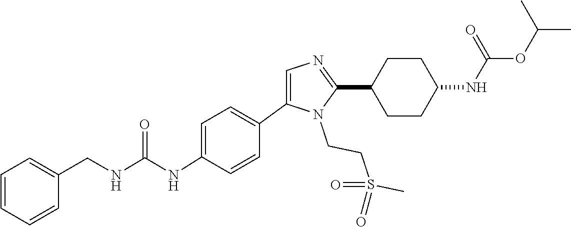 | isopropyl ((trans)-4-(5-(4-(3- benzylureido)phenyl)-1-(2- (methylsulfonyl)ethyl)-1H- imidazol-2- yl)cyclohexyl)carbamate |
| 34 | 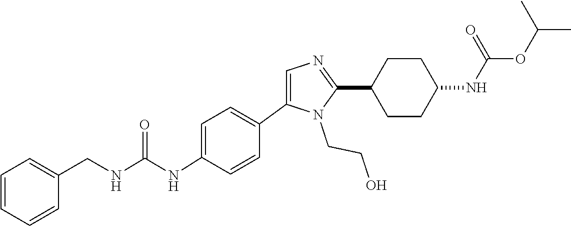 | isopropyl ((trans)-4-(5-(4-(3- benzylureido)phenyl)-1-(2- hydroxyethyl)-1H-imidazol-2- yl)cyclohexyl)carbamate |
| 35 | 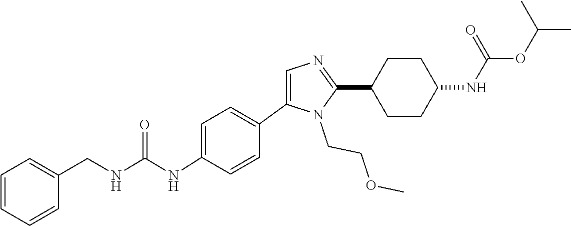 | isopropyl ((trans)-4-(5-(4-(3- benzylureido)phenyl)-1-(2- methoxyethyl)-1H-imidazol-2- yl)cyclohexyl)carbamate |
| 36 | 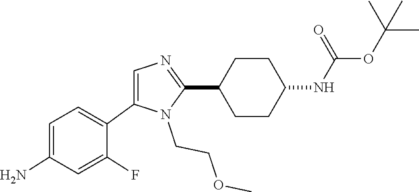 | tert-butyl ((trans)-4-(5-(4-amino- 2-fluorophenyl)-1-(2- methoxyethyl)-1H-imidazol-2- yl)cyclohexyl)carbamate |
| 37 | 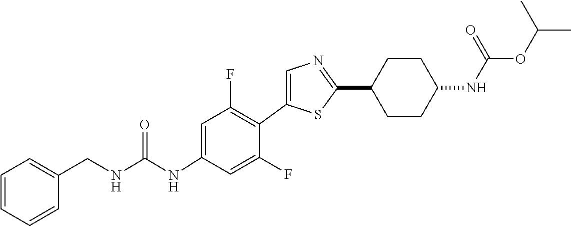 | isopropyl ((trans)-4-(5-(4-(3- benzylureido)-2,6- difluorophenyl)thiazol-2- yl)cyclohexyl)carbamate |
| 38 | 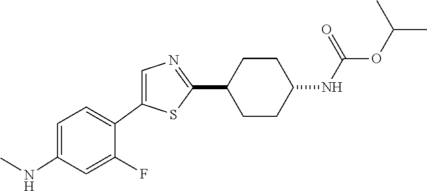 | isopropyl ((trans)-4-(5-(2-fluoro- 4-(methylamino)phenyl)thiazol- 2-yl)cyclohexyl)carbamate |
| 39 | 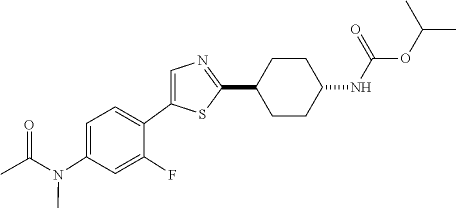 | isopropyl ((trans)-4-(5-(2-fluoro- 4-(N- methylacetamido)phenyl)thiazol- 2-yl)cyclohexyl)carbamate |
| 40 | 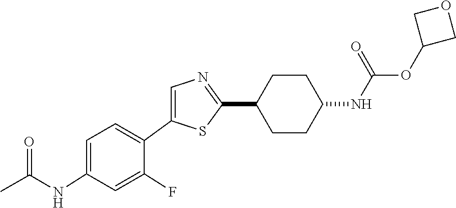 | oxetan-3-yl ((trans)-4-(5-(4- acetamido-2- fluorophenyl)thiazol-2- yl)cyclohexyl)carbamate |
| 41 | 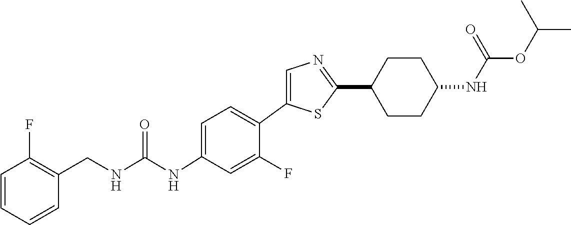 | isopropyl ((trans)-4-(5-(2-fluoro- 4-(3-(2- fluorobenzyl)ureido)phenyl) thiazol-2-yl)cyclohexyl)carbamate |
| 42 | 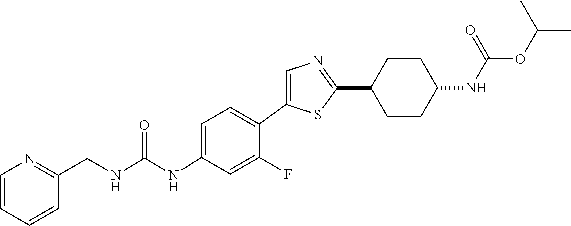 | isopropyl ((trans)-4-(5-(2-fluoro- 4-(3-(pyridin-2- ylmethyl)ureido)phenyl)thiazol- 2-yl)cyclohexyl)carbamate |
| 43 | 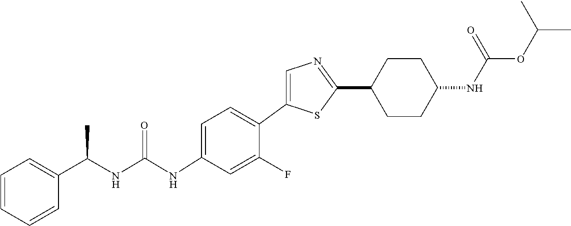 | isopropyl ((trans)-4-(5-(2-fluoro- 4-(3-((R)-1- phenylethyl)ureido)phenyl) thiazol-2-yl)cyclohexyl)carbamate |
| 44 | 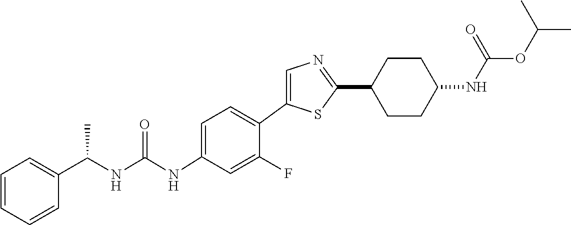 | isopropyl ((trans)-4-(5-(2-fluoro- 4-(3-((S)-1- phenylethyl)ureido)phenyl) thiazol-2-yl)cyclohexyl)carbamate |
| 45 | 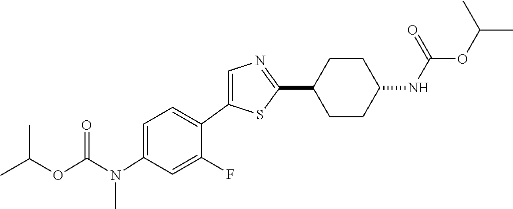 | isopropyl (3-fluoro-4-(2-((trans)- 4- ((isopropoxycarbonyl)amino) cyclohexyl)thiazol-5- yl)phenyl)(methyl)carbamate |
| 46 | 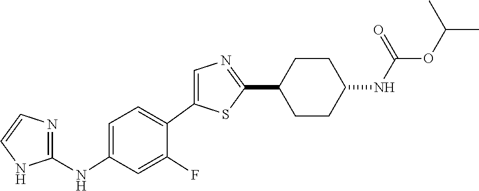 | isopropyl ((trans)-4-(5-(4-((1H- imidazol-2-yl)amino)-2- fluorophenyl)thiazol-2- yl)cyclohexyl)carbamate |
| 47 | 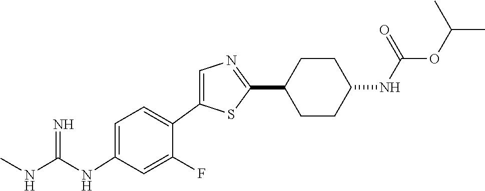 | isopropyl ((trans)-4-(5-(2-fluoro- 4-(3- methylguanidino)phenyl)thiazol- 2-yl)cyclohexyl)carbamate |
| 48 | 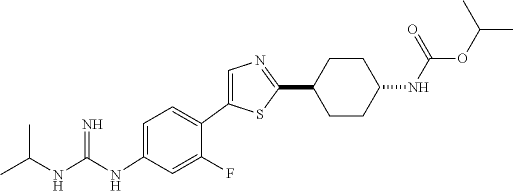 | isopropyl ((trans)-4-(5-(2-fluoro- 4-(3- isopropylguanidino)phenyl)thiazol- 2-yl)cyclohexyl)carbamate |
| 49 | 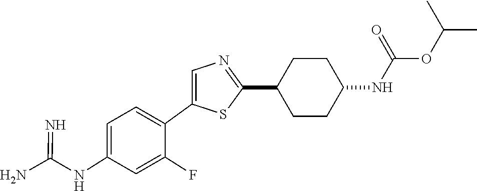 | isopropyl ((trans)-4-(5-(2-fluoro- 4-guanidinophenyl)thiazol-2- yl)cyclohexyl)carbamate |
| 50 | 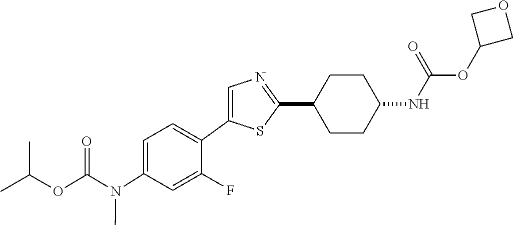 | isopropyl (3-fluoro-4-(2- ((transoo)-4-(((oxetan-3- yloxy)carbonyl)amino)cyclohexyl) thiazol-5- yl)phenyl)(methyl)carbamate |
| 51 | 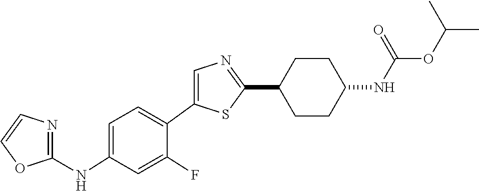 | isopropyl ((trans)-4-(5-(2-fluoro- 4-(oxazol-2- ylamino)phenyl)thiazol-2- yl)cyclohexyl)carbamate |
| 52 | 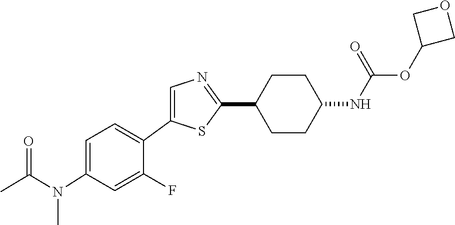 | oxetan-3-yl ((trans)-4-(5-(2- fluoro-4-(N- methylacetamido)phenyl)thiazol- 2-yl)cyclohexyl)carbamate |

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and pharmaceutically acceptable prodrugs, solvates, or salts thereof.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table 1 and pharmaceutically acceptable prodrugs, solvates, or salts thereof.

In some embodiments, the compound is a deuterium labeled compound of any one of the compounds described in Table 1 and pharmaceutically acceptable prodrugs, solvates, or salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognised techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the invention or a pharmaceutically acceptable salt or solvate thereof that contains the aforementioned deuterium atom(s) is within the scope of the invention. Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulphuric, phosphoric, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess MCT inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, glutamate, glucuronate, glutarate, malate, maleate, succinate, fumarate, tartrate, tosylate, salicylate, lactate, naphthalenesulphonate, and acetate.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess MCT inhibitory activity.

It is also to be understood that certain compounds of any one of the Formulae disclosed herein may exhibit polymorphism, and that the disclosure encompasses all such forms, or mixtures thereof, which possess MCT inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

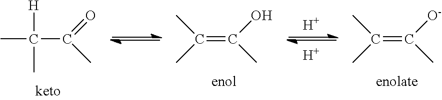

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the sulphonylurea group in a compound of the any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and H) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesised by any one of the processes defined herein, the processes may then further comprise the additional steps of: (i) removing any protecting groups present; (ii) converting the compound Formula (I) into another compound of Formula (I); (iii) forming a pharmaceutically acceptable salt, hydrate or solvate thereof, and/or (iv) forming a prodrug thereof.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

Compounds of Formula (I) can be prepared by generally known methods.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound or a pharmaceutically acceptable prodrug, solvate, or salt thereof of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intra-muscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavoring.

According to a further aspect of the disclosure there is provided a pharmaceutical composition which comprises a compound or a pharmaceutically acceptable prodrug, solvate, or salt thereof of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound or a pharmaceutically acceptable prodrug, solvate, or salt thereof of the present disclosure for use in therapy is an amount sufficient to treat or prevent an MCT related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or patient and the route of administration, according to well-known principles of medicine.

### Methods of Use

In some aspects, the present disclosure provides a method of modulating MCT (e.g., the MCT1) activity (e.g., in vitro or in vivo), comprising contacting a cell with an effective amount of a compound of the present disclosure or a pharmaceutically acceptable prodrug, solvate, or salt thereof.

In some aspects, the present disclosure provides a method of modulating MCT (e.g., the MCT1) activity (e.g., in vitro or in vivo), comprising contacting a cell with a compound of the present disclosure or a pharmaceutically acceptable prodrug, solvate, or salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating or preventing a disease or disorder disclosed herein in a subject in need thereof, comprising administering to the subject a compound of the present disclosure or a pharmaceutically acceptable prodrug, solvate, or salt thereof, or a pharmaceutical composition of the present disclosure.

In some embodiments, the disease or disorder is associated with an implicated MCT activity. In some embodiments, the disease or disorder is a disease or disorder in which MCT activity is implicated.

In some embodiments, the disease or disorder is associated with an implicated MCT1 activity. In some embodiments, the disease or disorder is a disease or disorder in which MCT1 activity is implicated.

In some embodiments, the disease or disorder is associated with an implicated MCT4 activity. In some embodiments, the disease or disorder is a disease or disorder in which MCT4 activity is implicated.

In some embodiments, the disease or disorder is a cancer, an autoimmune disease, an immune deficiency, or a neurodegenerative disease.

In some aspects, the present disclosure provides a method of treating or preventing a cancer or a neurodegenerative disease in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating or preventing a cancer or a neurodegenerative disease in a subject in need thereof, comprising administering to the subject a compound of the present disclosure or a pharmaceutically acceptable prodrug, solvate, or salt thereof, or a pharmaceutical composition of the present disclosure.

In some aspects, the present disclosure provides a method of treating a cancer or a neurodegenerative disease in a subject in need thereof, comprising administering to the subject a therapeutically effective amount of a compound of the present disclosure or a pharmaceutically acceptable prodrug, solvate, or salt thereof, or a pharmaceutical composition of the present disclosure.

In some aspects, the present disclosure provides a method of treating a cancer or a neurodegenerative disease in a subject in need thereof, comprising administering to the subject a compound of the present disclosure or a pharmaceutically acceptable prodrug, solvate, or salt thereof, or a pharmaceutical composition of the present disclosure.

In some aspects, the present disclosure provides a compound of the present disclosure or a pharmaceutically acceptable prodrug, solvate, or salt thereof for use in modulating MCT (e.g., the MCT1) activity (e.g., in vitro or in vivo).

In some aspects, the present disclosure provides a compound of the present disclosure or a pharmaceutically acceptable prodrug, solvate, or salt thereof for use in treating or preventing a disease or disorder disclosed herein.

In some aspects, the present disclosure provides a compound of the present disclosure or a pharmaceutically acceptable prodrug, solvate, or salt thereof for use in treating a disease or disorder disclosed herein.

In some aspects, the present disclosure provides a compound of the present disclosure or a pharmaceutically acceptable prodrug, solvate, or salt thereof for use in treating or preventing a cancer or a neurodegenerative disease in a subject in need thereof.

In some aspects, the present disclosure provides a compound of the present disclosure or a pharmaceutically acceptable prodrug, solvate, or salt thereof for use in treating a cancer or a neurodegenerative disease in a subject in need thereof.

In some aspects, the present disclosure provides use of a compound of the present disclosure or a pharmaceutically acceptable prodrug, solvate, or salt thereof in the manufacture of a medicament for modulating MCT (e.g., the MCT1) activity (e.g., in vitro or in vivo).

In some aspects, the present disclosure provides use of a compound of the present disclosure or a pharmaceutically acceptable prodrug, solvate, or salt thereof in the manufacture of a medicament for treating or preventing a disease or disorder disclosed herein.

In some aspects, the present disclosure provides use of a compound of the present disclosure or a pharmaceutically acceptable prodrug, solvate, or salt thereof in the manufacture of a medicament for treating a disease or disorder disclosed herein.

In some aspects, the present disclosure provides use of a compound of the present disclosure or a pharmaceutically acceptable prodrug, solvate, or salt thereof in the manufacture of a medicament for treating or preventing a cancer or a neurodegenerative disease in a subject in need thereof.

In some aspects, the present disclosure provides use of a compound of the present disclosure or a pharmaceutically acceptable prodrug, solvate, or salt thereof in the manufacture of a medicament for treating a cancer or a neurodegenerative disease in a subject in need thereof.

In some embodiments, the disease or disorder is a cancer, an autoimmune disease, an immune deficiency, or a neurodegenerative disease.

In some embodiments, the cancer to be treated is a B-cell neoplasm.

In some embodiments, the cancer is selected from the group consisting of lymphoma, leukemia, and a plasma cell neoplasm. In some embodiments, the cancer selected from the group consisting of carcinoma and sarcoma.

In some embodiments, the cancer to be treated is a lymphoma. Lymphomas which can be treated by the disclosed methods include Non-Hodgkin's lymphoma; Burkitt's lymphoma; small lymphocytic lymphoma; lymphoplasmacytic lymphoma; MALT lymphoma; follicular lymphoma; diffuse large B-cell lymphoma; and T-cell lymphoma.

In some embodiments, leukemias which can be treated by the disclosed methods include acute lymphoblastic leukemia (ALL); Burkitt's leukemia; B-cell leukemia; B-cell acute lymphoblastic leukemia; chronic lymphocytic leukemia (CLL); acute myelogenous leukemia (AML); chronic myelogenous leukemia (CML); and T-cell acute lymphoblastic leukemia (T-ALL).

In some embodiments the cancer to be treated is B-cell neoplasms, B-cell leukemia, B-cell acute lymphoblastic leukemia, chronic lymphocytic leukemia, chronic myelogenous leukemia, Burkitt's leukemia, acute myelogenous leukemia and/or T-ALL. In some embodiments the cancer to be treated is chronic lymphocytic leukemia (CLL) or chronic myelogenous leukemia (CML).

In some embodiments, the cancer to be treated is a plasma cell neoplasm. Examples for plasma cell neoplasms include multiple myeloma; plasma cell myeloma; plasma cell leukemia and plasmacytoma.

Carcinomas which can be treated by the disclosed methods include colon cancer; liver cancer; gastric cancer; intestinal cancer; esophageal cancer; breast cancer; ovarian cancer; head and neck cancer; lung cancer; and thyroid cancer.

Sarcomas which can be treated by the disclosed methods include soft tissue sarcoma and bone sarcoma.

In some embodiments, the cancer that can be treated by the disclosed methods include cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; sarcomas; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the disease or disorder is Lynch syndrome.

Lynch syndrome is a hereditary disorder caused by a mutation in a mismatch repair gene in which affected individuals have a higher than normal chance of developing colorectal cancer, endometrial cancer, and various other types of aggressive cancers, often at a young age—also called hereditary nonpolyposis colon cancer (HNPCC). The mutations of specific mismatch repair (MMR) genes including but not limited to MLH1, MSH2, MSH6, PMS2, and EPCAM-TACSTD1 deletions are responsible for Lynch syndrome. These genes work in repairing mistakes made when DNA is copied in preparation for cell division. The defects in the genes disallow repair of DNA mistakes and as cells divide, errors stack and uncontrollable cell growth may result in cancer. Those with Lynch syndrome carry up to an 85% risk of contracting colon cancer as well as a higher than average risk for endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

Thus, in one embodiment for the disclosed method, the method is a method of treating cancer derived from Lynch syndrome, selected from the group consisting of colon cancer, endometrial cancer, stomach cancer, pancreatic cancer, kidney/ureter tract cancer, hepatobiliary tract cancer, gastric tract cancer, prostate cancer, ovarian cancer, gallbladder duct cancer, brain cancer, small intestine cancer, breast cancer, and skin cancer.

In some embodiments, the neurodegenerative disorder is selected from the group consisting of multiple sclerosis, Parkinson's disease (PD), Alzheimer's disease (AD), Dentatorubropallidoluysian atrophy (DRPLA), Huntington's Disease (HD), Spinocerebellar ataxia Type 1 (SCA1), Spinocerebellar ataxia Type 2 (SCA2), Spinocerebellar ataxia Type 3 (SCA3), Spinocerebellar ataxia 6 (SCA6), Spinocerebellar ataxia Type 7 (SCA7), Spinocerebellar ataxia Type 8 (SCA8), Spinocerebellar ataxia Type 12 (SCA12), Spinocerebellar ataxia Type 17 (SCA17), Spinobulbar Muscular Ataxia/Kennedy Disease (SBMA), Fargile X syndrome (FRAXA), Fragile XE mental retardation (FRAXE), and Myotonic dystrophy (DM).

The present disclosure provides a compound that functions as modulator of MCT activity. The present disclosure therefore provides a method of modulating MCT activity in vitro or in vivo, said method comprising contacting a cell with an effective amount of a compound, or a pharmaceutically acceptable salt thereof, as defined herein.

In some aspects, the present disclosure provides a method of treating or preventing a disease or disorder associated with the abnormal expression or activity of monocarboxylate transporters (MCTs), or dependency on the expression or activity of at least one MCT, wherein the method comprises administering to a subject in need thereof a compound or a pharmaceutically acceptable prodrug, solvate, or salt thereof of the present disclosure, or a pharmaceutical composition thereof.

In some aspects, the present disclosure provides a method of treating or preventing a disease or disorder, wherein the method comprises administering to a subject in need thereof a compound or a pharmaceutically acceptable prodrug, solvate, or salt thereof of the present disclosure, or a pharmaceutical composition thereof, and
wherein the compound is administered in a therapeutically effective amount to modulate the activity of monocarboxylate transporters (MCTs).

In some aspects, the present disclosure provides a method of treating or preventing a disease or disorder, wherein the method comprises:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides the use of a compound or a pharmaceutically acceptable prodrug, solvate, or salt thereof of a compound or the present disclosure, or a pharmaceutical composition thereof, in the manufacture of a medicament for treating or preventing a disease or disorder associated with the abnormal expression or activity of monocarboxylate transporters (MCTs), or dependency on the expression or activity of at least one MCT.

In some aspects, the present disclosure provides the use of a compound or a pharmaceutically acceptable prodrug, solvate, or salt thereof of the present disclosure, or the pharmaceutical composition thereof, in the manufacture of a medicament for treating or preventing a disease or disorder, and wherein the compound is administered in a therapeutically effective amount to modulate the activity of monocarboxylate transporters (MCTs).

In some aspects, the present disclosure provides the use of a compound or a pharmaceutically acceptable prodrug, solvate, or salt thereof of the present disclosure, or a pharmaceutical composition thereof, in the manufacture of a medicament for treating or preventing a disease or disorder comprising:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides the use of a compound or a pharmaceutically acceptable prodrug, solvate, or salt thereof of the present disclosure, or a pharmaceutical composition thereof, for treating or preventing a disease or disorder associated with the abnormal expression or activity of monocarboxylate transporters (MCTs), or dependency on the expression or activity of at least one MCT.

In some aspects, the present disclosure provides the use of a compound or a pharmaceutically acceptable prodrug, solvate, or salt thereof of the present disclosure, or a pharmaceutical composition thereof, for treating or preventing a disease or disorder, and
wherein the compound is administered in a therapeutically effective amount to modulate the activity of monocarboxylate transporters (MCTs).

In some aspects, the present disclosure provides the use of a compound or a pharmaceutically acceptable prodrug, solvate, or salt thereof of the present disclosure, or a pharmaceutical composition thereof, for treating or preventing a disease or disorder comprising:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some aspects, the present disclosure provides a compound or a pharmaceutically acceptable prodrug, solvate, or salt thereof of the present disclosure, or a pharmaceutical composition thereof, for use in treating or preventing a disease or disorder associated with the abnormal expression or activity of monocarboxylate transporters (MCTs), or dependency on the expression or activity of at least one MCT.

In some aspects, the present disclosure provides a compound or a pharmaceutically acceptable prodrug, solvate, or salt thereof of the present disclosure, or a pharmaceutical composition thereof, for use in treating or preventing a disease or disorder, and
wherein the compound is administered in a therapeutically effective amount to modulate the activity of monocarboxylate transporters (MCTs).

In some aspects, the present disclosure provides a compound or a pharmaceutically acceptable prodrug, solvate, or salt thereof of the present disclosure, or a pharmaceutical composition thereof, for use in treating or preventing a disease or disorder comprising:
a. identifying a subject with an abnormal expression or activity of at least one MCT, or dependency on the expression or activity of at least one MCT; and
b. administering to the subject a compound or a pharmaceutically acceptable prodrug, solvate, or salt thereof of the present disclosure, or a pharmaceutical composition thereof.

In some embodiments, the MCT is MCT1.

In some embodiments, the MCT is MCT4.

In some embodiments, the expression or activity of the MCT is increased.

In some embodiments, the expression or activity of the MCT is decreased.

In some embodiments, the expression or activity of MCT1 is increased.

In some embodiments, the expression or activity of MCT4 is decreased.

In some embodiments, the MCT activity of a compound or a pharmaceutically acceptable prodrug, solvate, or salt thereof of the present disclosure is assessed using a lactate transporter assay.

In some embodiments, the disease or disorder is cancer.

In some embodiments, the cancer is a MCT1 high-expressing cancer.

In some embodiments, the cancer is a lymphoma or a solid tumor.

In some embodiments, the compound of the present disclosure inhibits the activity of MCT1 with an IC₅₀ from about 5 nM to about 1000 nM.

In some embodiments, the compound of the present disclosure inhibits the activity of MCT1 with an IC₅₀ of about 5 nM.

In some embodiments, the compound of the present disclosure inhibits the activity of MCT1 with an IC₅₀ of about 10 nM.

In some embodiments, the compound of the present disclosure inhibits the activity of MCT1 with an IC₅₀ of about 20 nM.

In some embodiments, the compound of the present disclosure inhibits the activity of MCT1 with an IC₅₀ of about 30 nM.

In some embodiments, the compound of the present disclosure inhibits the activity of MCT1 with an IC₅₀ of about 40 nM.

In some embodiments, the compound of the present disclosure inhibits the activity of MCT1 with an IC₅₀ of about 50 nM.

In some embodiments, the compound of the present disclosure inhibits the activity of MCT1 with an IC₅₀ of about 100 nM.

In some embodiments, the compound of the present disclosure inhibits the activity of MCT1 with an IC₅₀ of about 150 nM.

In some embodiments, the compound of the present disclosure inhibits the activity of MCT1 with an IC₅₀ of about 200 nM.

In some embodiments, the compound of the present disclosure inhibits the activity of MCT1 with an IC₅₀ of about 250 nM.

In some embodiments, the compound of the present disclosure inhibits the activity of MCT1 with an IC₅₀ of about 300 nM.

In some embodiments, the compound of the present disclosure inhibits the activity of MCT1 with an IC₅₀ of about 350 nM.

In some embodiments, the compound of the present disclosure inhibits the activity of MCT1 with an IC₅₀ of about 400 nM.

In some embodiments, the compound of the present disclosure inhibits the activity of MCT1 with an IC₅₀ of about 450 nM.

In some embodiments, the compound of the present disclosure inhibits the activity of MCT1 with an IC₅₀ of about 500 nM.

In some embodiments, the compound of the present disclosure inhibits the activity of MCT1 with an IC₅₀ of about 550 nM.

In some embodiments, the compound of the present disclosure inhibits the activity of MCT1 with an IC₅₀ of about 600 nM.

In some embodiments, the compound of the present disclosure inhibits the activity of MCT1 with an IC₅₀ of about 650 nM.

In some embodiments, the compound of the present disclosure inhibits the activity of MCT1 with an IC₅₀ of about 700 nM.

In some embodiments, the compound of the present disclosure inhibits the activity of MCT1 with an IC₅₀ of about 750 nM.

In some embodiments, the compound of the present disclosure inhibits the activity of MCT1 with an IC₅₀ of about 800 nM.

In some embodiments, the compound of the present disclosure inhibits the activity of MCT1 with an IC₅₀ of about 850 nM.

In some embodiments, the compound of the present disclosure inhibits the activity of MCT1 with an IC₅₀ of about 900 nM.

In some embodiments, the compound of the present disclosure inhibits the activity of MCT1 with an IC₅₀ of about 950 nM.

In some embodiments, the compound of the present disclosure inhibits the activity of MCT1 with an IC₅₀ of about 1000 nM.

In some embodiments, the intracellular lactate accumulation with compounds of the present disclosure has an EC₅₀ from about 5 nM to about 1000 nM.

In some embodiments, the intracellular lactate accumulation with compounds of the present disclosure has an EC₅₀ of about 5 nM.

In some embodiments, the intracellular lactate accumulation with compounds of the present disclosure has an EC₅₀ of about 10 nM.

In some embodiments, the intracellular lactate accumulation with compounds of the present disclosure has an EC₅₀ of about 20 nM.

In some embodiments, the intracellular lactate accumulation with compounds of the present disclosure has an EC₅₀ of about 30 nM.

In some embodiments, the intracellular lactate accumulation with compounds of the present disclosure has an EC₅₀ of about 40 nM.

In some embodiments, the intracellular lactate accumulation with compounds of the present disclosure has an EC₅₀ of about 50 nM.

In some embodiments, the intracellular lactate accumulation with compounds of the present disclosure has an EC₅₀ of about 100 nM.

In some embodiments, the intracellular lactate accumulation with compounds of the present disclosure has an EC₅₀ of about 150 nM.

In some embodiments, the intracellular lactate accumulation with compounds of the present disclosure has an EC₅₀ of about 200 nM.

In some embodiments, the intracellular lactate accumulation with compounds of the present disclosure has an EC₅₀ of about 250 nM.

In some embodiments, the intracellular lactate accumulation with compounds of the present disclosure has an EC₅₀ of about 300 nM.

In some embodiments, the intracellular lactate accumulation with compounds of the present disclosure has an EC₅₀ of about 350 nM.

In some embodiments, the intracellular lactate accumulation with compounds of the present disclosure has an EC₅₀ of about 400 nM.

In some embodiments, the intracellular lactate accumulation with compounds of the present disclosure has an EC₅₀ of about 450 nM.

In some embodiments, the intracellular lactate accumulation with compounds of the present disclosure has an EC₅₀ of about 500 nM.

In some embodiments, the intracellular lactate accumulation with compounds of the present disclosure has an EC₅₀ of about 550 nM.

In some embodiments, the intracellular lactate accumulation with compounds of the present disclosure has an EC₅₀ of about 600 nM.

In some embodiments, the intracellular lactate accumulation with compounds of the present disclosure has an EC₅₀ of about 650 nM.

In some embodiments, the intracellular lactate accumulation with compounds of the present disclosure has an EC₅₀ of about 700 nM.

In some embodiments, the intracellular lactate accumulation with compounds of the present disclosure has an EC₅₀ of about 750 nM.

In some embodiments, the intracellular lactate accumulation with compounds of the present disclosure has an EC₅₀ of about 800 nM.

In some embodiments, the intracellular lactate accumulation with compounds of the present disclosure has an EC₅₀ of about 850 nM.

In some embodiments, the intracellular lactate accumulation with compounds of the present disclosure has an EC₅₀ of about 900 nM.

In some embodiments, the intracellular lactate accumulation with compounds of the present disclosure has an EC₅₀ of about 950 nM.

In some embodiments, the intracellular lactate accumulation with compounds of the present disclosure has an EC₅₀ of about 1000 nM.

Effectiveness of compounds of the disclosure can be determined by industry-accepted assays/disease models according to standard practices of elucidating the same as described in the art and are found in the current general knowledge.

The present disclosure also provides a method of treating a disease or disorder in which MCT activity is implicated in a patient in need of such treatment, said method comprising administering to said patient a therapeutically effective amount of a compound, or a pharmaceutically acceptable prodrug, solvate, or salt thereof, or a pharmaceutical composition as defined herein.

Suitably, the compounds according to the present disclosure can be used for the treatment of a disease selected from a cancer, an autoimmune disease, an immune deficiency, or a neurodegenerative disease.

Compounds of the present disclosure, or pharmaceutically acceptable prodrugs, solvates, or salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which MCT activity is implicated comprising a compound of the disclosure as defined hereinbefore, or a pharmaceutically acceptable prodrug, solvate, or salt thereof, and another suitable agent.

According to a further aspect of the disclosure there is provided a pharmaceutical composition which comprises a compound of the disclosure, or a pharmaceutically acceptable prodrug, solvate, or salt thereof, in combination with a suitable therapeutic agent, in association with a pharmaceutically acceptable diluent or carrier.

In addition to its use in therapeutic medicine, compounds of Formula (I) and pharmaceutically acceptable prodrugs, solvates, or salts thereof are also useful as pharmacological tools in the development and standardisation of in vitro and in vivo test systems for the evaluation of the effects of inhibitors of MCT in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, such depiction also refers to the various neutral forms, cationic forms, and anionic forms of the compound. For another example, when a compound the present disclosure is depicted in an anionic form, such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₅). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 6-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic.

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The terms “heterocyclyl”, “heterocyclic ring”, and “heterocyclic group”, are used interchangeably herein, and means saturated or unsaturated non-aromatic 3-10 membered ring radical containing from 1 to 4 ring heteroatoms, which may be the same or different, selected from N, O, or S. It can be monocyclic, bicyclic or tricyclic (e.g., a fused or bridged bicyclic or tricyclic ring). Examples of include, but are not limited to, azetidinyl, morpholinyl, thiomorpholinyl, pyrrolidinonyl, pyrrolidinyl, piperidinyl, piperazinyl, hydantoinyl, valerolactamyl, oxiranyl, oxetanyl, dihydroimidazole, dihydrofuranyl, dihydropyranyl, dihydropyridinyl, dihydropyrimidinyl, dihydrothienyl, dihydrothiophenyl, dihydrothiopyranyl, tetrahydroimidazole, tetrahydrofuranyl, tetrahydropyranyl, tetrahydrothienyl, tetrahydropyridinyl, tetrahydropyrimidinyl, tetrahydrothiophenyl, and tetrahydrothiopyranyl. A heterocyclic ring optionally contains one or more double bonds and/or is optionally fused with one or more aromatic rings (for example, tetrahydronaphthyridine, indolinone, dihydropyrrolotriazole, imidazopyrimidine, quinolinone, dioxaspirodecane). Examples of 3-7 membered monocyclic heterocyclic ring include, but are not limited to, azetidinyl, morpholinyl, thiomorpholinyl, pyrrolidinonyl, pyrrolidinyl, piperidinyl, piperazinyl, hydantoinyl, valerolactamyl, oxiranyl, oxetanyl, dihydroimidazole, dihydrofuranyl, dihydropyranyl, dihydropyridinyl, dihydropyrimidinyl, dihydrothienyl, dihydrothiophenyl, dihydrothiopyranyl, tetrahydroimidazole, tetrahydrofuranyl, tetrahydropyranyl, tetrahydrothienyl, tetrahydropyridinyl, tetrahydropyrimidinyl, tetrahydrothiophenyl, and tetrahydrothiopyranyl.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated those compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J, March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who has (e.g., is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that doesn't respond or hasn't yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable prodrug, solvate, or salt thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable prodrug, solvate, or salt thereof, can or may also be used to prevent a relevant disease, condition, or disorder, or used to identify suitable candidates for such purposes.

The terms “inhibiting”, “reducing”, or any variation of these terms in relation of MCT, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of MCT activity compared to its normal activity.

As used herein, the term “preventing,” “prevent,” or “protecting against” describes reducing or eliminating the onset of the symptoms or complications of such disease, condition or disorder.

The term “disorder” is used in this disclosure to mean, and is used interchangeably with, the terms disease, condition, or illness, unless otherwise indicated.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches, and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable prodrugs, solvates, or salt include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable prodrugs, solvates, or salt thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### EXAMPLES

For exemplary purpose, neutral compounds of Formula (I) are synthesized and tested in the examples. It is understood that salts of the compounds of Formula (I) may be similarly synthesized and tested using the exemplary procedures described in the examples. Further, it is understood that the neutral compounds of the compounds of Formula (I) may be converted to the corresponding salt of the compounds of Formula (I) using routine techniques in the art (e.g., pH adjustment and, optionally, extraction).

Compounds of Formula (I) can be prepared using the methods detailed herein. Those skilled in the art may be able to envisage alternative synthetic routes, using a variety of starting materials and reagents to prepare the disclosed compounds of Formula (I) and to make further modifications.

### Abbreviations

Ac acetyl
ACN acetonitrile
aq aqueous
Bn benzyl
Boc tert-butoxycarbonyl
br. broad
d doublet (only when used within 1H NMR spectra)
DCM dichloromethane
DIEA(DIPEA) diisopropylethylamine
DMA dimethylacetamide
DMAP 4-dimethylaminopyridine
DMF N,N-dimethylformamide
DMSO dimethylsulfoxide
dppf 1,1′-bis(diphenylphosphino) ferrocene
eq equivalent
EtOAc ethyl acetate
hr hour
HBTU N,N,N′,N′-tetramethyl-O-(1H-benzotriazol-1-yl)uronium hexafluorophosphate
HPLC high performance liquid chromatography
LC-MS liquid chromatography coupled to mass spectrometry
m multiplet
MS ESI mass spectra, electrospray ionization
NBS N-bromosuccinimide
NMR nuclear magnetic resonance
prep preparative
Py pyridine
s singlet
sat saturated
SFC supercritical fluid chromatography
t triplet
TEA triethylamine
TFA trifluoroacetic acid
THF tetrahydrofuran
TLC thin layer chromatography
Tol toluene

### Example 1: Preparation of isopropyl ((trans)-4-(5-(4-((isopropoxycarbonyl)amino)-2-morpholinophenyl)thiazol-2-yl)cyclohexyl)carbamate

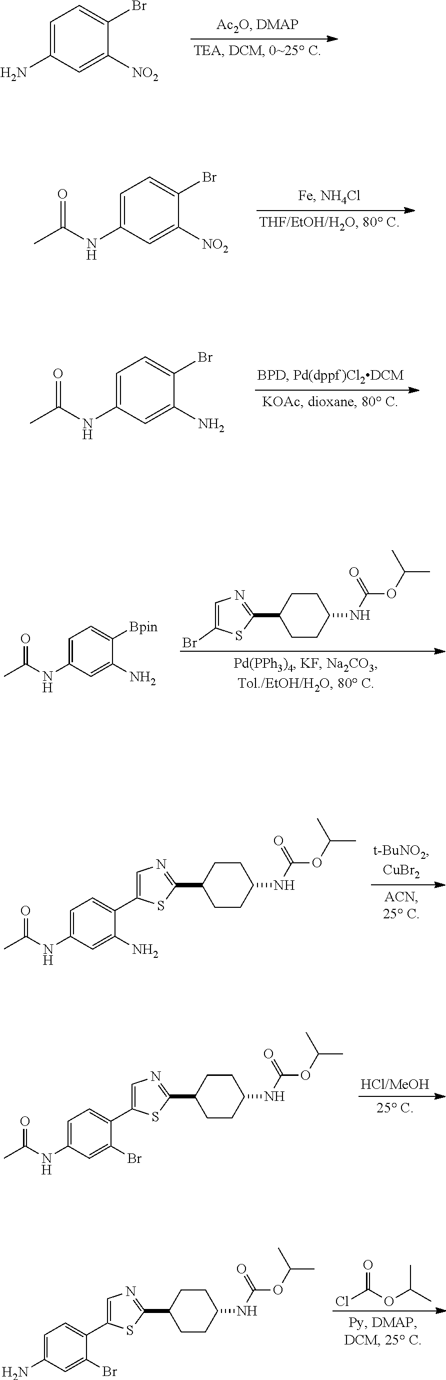

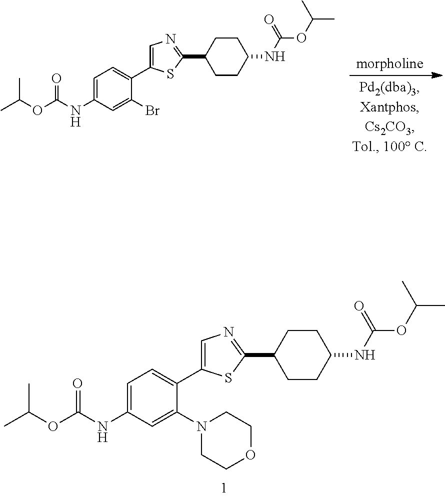

### Step 1: Synthesis of N-(4-bromo-3-nitrophenyl)acetamide

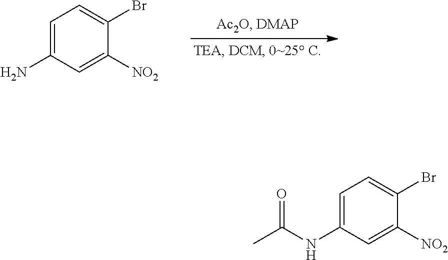

To a solution of 4-bromo-3-nitro-aniline (25.0 g, 115 mmol, 1.0 eq.) in DCM (250 mL) added DMAP (1.4 g, 12 mmol, 0.1 eq.), TEA (35.0 g, 346 mmol, 3.0 eq.) and acetyl acetate (11.8 g, 115 mmol, 1.0 eq.) at 0° C. The mixture was stirred at 25° C. for 1 hr, quenched by 1N HCl (500 mL) and extracted with DCM (800 mL×3). The combined organic layers were washed with sat. aq. Na₂CO₃ (100 mL), dried over Na₂SO₄, filtered, concentrated and triturated with Petroleum ether:EtOAc=2:1 (200 mL) to yield N-(4-bromo-3-nitro-phenyl)acetamide (24.0 g, 93 mmol, 80.4% yield) as a yellow solid.

¹H NMR (400 MHz, methanol-d4) δ=8.28 (d, J=2.6 Hz, 1H), 7.70 (d, J=8.8 Hz, 1H), 7.59 (dd, J=2.6, 8.8 Hz, 1H), 2.14 (s, 3H). ESI [M+H]=261.1/259.1

### Step 2: Synthesis of N-(3-amino-4-bromophenyl)acetamide

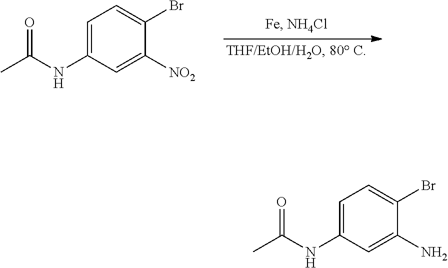

To a solution of N-(4-bromo-3-nitro-phenyl)acetamide (24.0 g, 93 mmol, 1.0 eq.) in THF (60 mL)/EtOH (120 mL)/H₂O (60 mL) was added powdered Fe (25.9 g, 463 mmol, 5.0 eq.) and NH₄Cl (14.9 g, 278 mmol, 3.0 eq.). The mixture was stirred at 80° C. for 3 hrs, filtered and concentrated to remove solvent. The residue was diluted with H₂O (500 mL) and extracted with EtOAc (800 mL×3). The combined organic layers were dried over Na₂SO₄, filtered and concentrated to yield N-(3-amino-4-bromo-phenyl)acetamide (19.4 g, crude) as a yellow solid. ¹H NMR (400 MHz, methanol-d4) δ=7.30-7.13 (m, 2H), 6.67 (dd, J=2.4, 8.6 Hz, 1H), 2.08 (s, 3H). ESI [M+H]=229.0/231.0

### Step 3: Synthesis of N-(3-amino-4-(4,4,5,5-tetramethyl-1,3,2-dioxaborolan-2-yl)phenyl)acetamide

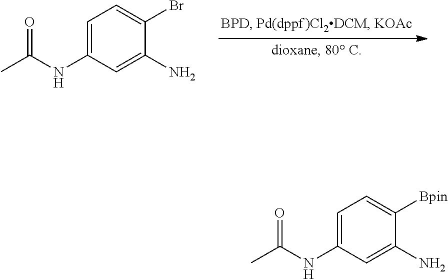

A mixture of N-(3-amino-4-bromo-phenyl)acetamide (19.0 g, 83 mmol, 1.0 eq.), 4,4,4′,4′,5,5,5′,5′-octamethyl-2,2′-bi(1,3,2-dioxaborolane) (BPD) (63.2 g, 249 mmol, 3.0 eq.), KOAc (24.4 g, 249 mmol, 3.0 eq.) and Pd(dppf)Cl₂·CH₂Cl₂ (6.8 g, 8 mmol, 0.1 eq.) in dioxane (500 mL) was stirred at 80° C. for 12 hrs under N₂ atmosphere, concentrated and purified by column chromatography (SiO₂, Petroleum ether:EtOAc=100:1 to 3:1) to yield N-[3-amino-4-(4,4,5,5-tetramethyl-1,3,2-dioxaborolan-2-yl)phenyl]acetamide (10.5 g, 38 mmol, 45.8% yield) as a yellow oil. ¹H NMR (400 MHz, DMSO-d6) δ=9.70 (s, 1H), 7.23 (d, J=7.9 Hz, 1H), 6.98 (d, J=1.5 Hz, 1H), 6.59 (dd, J=1.8, 8.2 Hz, 1H), 5.49 (s, 2H), 2.05-1.91 (m, 3H), 1.25 (s, 12H). ESI [M+H]=277.2

### Step 4: Synthesis of trans-isopropyl N-[4-[5-(4-acetamido-2-amino-phenyl)thiazol-2-yl]cyclohexyl]carbamate

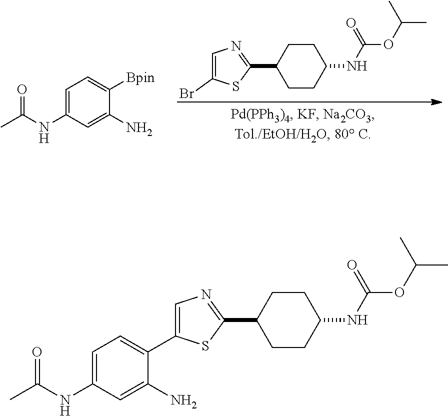

A mixture of N-[3-amino-4-(4,4,5,5-tetramethyl-1,3,2-dioxaborolan-2-yl)phenyl]-acetamide (10.5 g, 38 mmol, 1.2 eq.), trans-isopropyl N-[4-(5-bromothiazol-2-yl)cyclohexyl]carbamate (11.0 g, 32 mmol, 1.0 eq.), Pd(PPh₃)₄ (3.7 g, 3 mmol, 0.1 eq.), KF (2.8 g, 48 mmol, 1.5 eq.) and Na₂CO₃ (10.1 g, 95 mmol, 3.0 eq.) in EtOH (90 mL)/Tol. (90 mL)/H₂O (30 mL) was stirred at 80° C. for 12 hrs under N₂ atmosphere and concentrated to remove solvent. The residue was diluted with H₂O (300 mL), extracted with EtOAc (400 mL×3), dried over Na₂SO₄ and purified by column chromatography (SiO₂, Petroleum ether:EtOAc=30:1 to 0:1) to yield trans-isopropyl N-[4-[5-(4-acetamido-2-amino-phenyl)thiazol-2-yl]cyclohexyl]carbamate (13.0 g, 31 mmol, 98.5% yield) as a yellow solid. ¹H NMR (400 MHz, methanol-d4) δ=7.72-7.61 (m, 1H), 7.23 (d, J=2.1 Hz, 1H), 7.12 (d, J=8.3 Hz, 1H), 6.90-6.82 (m, 1H), 4.87-4.78 (m, 1H), 3.55-3.41 (m, 1H), 2.99 (tt, J=3.4, 12.1 Hz, 1H), 2.26-2.17 (m, 2H), 2.13 (s, 3H), 2.11-2.02 (m, 2H), 1.70 (dq, J=2.9, 12.8 Hz, 2H), 1.47-1.36 (m, 2H), 1.31-1.21 (m, 6H).

ESI [M+H]=417.2

### Step 5: Synthesis of trans-isopropyl N-[4-[5-(4-acetamido-2-bromo-phenyl)thiazol-2-yl]cyclohexyl]carbamate

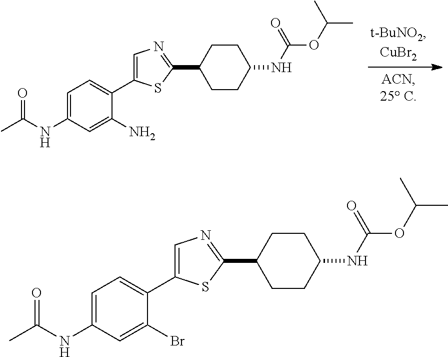

A mixture of trans-isopropyl N-[4-[5-(4-acetamido-2-amino-phenyl)thiazol-2-yl]cyclohexyl]carbamate (1.0 g, 2 mmol, 1.0 eq.), tert-butyl nitrite (371 mg, 4 mmol, 1.5 eq.) and CuBr₂ (187 mg, 840 umol, 0.4 eq.) in ACN (6 mL) was stirred at 25° C. for 0.5 hr under N₂ atmosphere, concentrated, diluted with H₂O (30 mL) and extracted with EtOAc (100 mL×3). The combined organic layers were dried over Na₂SO₄, filtered, concentrated and purified by column chromatography (SiO₂, Petroleum ether:EtOAc=1:1 to 0:1) to yield trans-isopropyl N-[4-[5-(4-acetamido-2-bromo-phenyl)thiazol-2-yl]cyclohexyl]carbamate (1.0 g, crude) as a yellow solid. ESI [M+H]=482.1/480.2

### Step 6: Synthesis of trans-isopropyl N-[4-[5-(4-amino-2-bromo-phenyl)thiazol-2-yl]cyclohexyl]carbamate

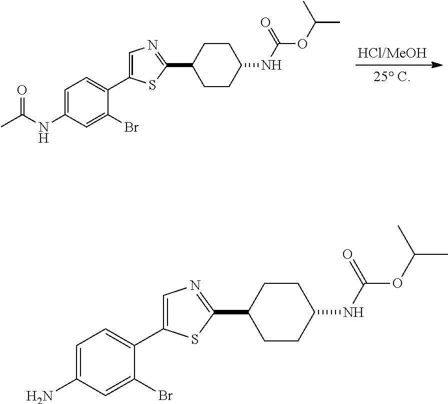 [SMILES: CC(=O)Nc1ccc(-c2cnc([C@H]3CC[C@H](NC(=O)OC(C)C)CC3)s2)c(Br)c1 ; CC(C)OC(=O)N[C@H]1CC[C@H](c2ncc(-c3ccc(N)cc3Br)s2)CC1]

A mixture of trans-isopropyl N-[4-[5-(4-acetamido-2-bromo-phenyl)thiazol-2-yl]cyclohexyl]carbamate (1.0 g, 2 mmol, 1.0 eq.) in 4M HCl/MeOH (50 mL) was stirred at 25° C. for 1 hr and concentrated to yield trans-isopropyl N-[4-[5-(4-amino-2-bromo-phenyl)thiazol-2-yl]cyclohexyl]carbamate (1.0 g, crude) as a yellow solid. ESI [M+H]=438.1/440.1

### Step 7: Synthesis of trans-isopropyl N-[3-bromo-4-[2-[4-(isopropoxycarbonylamino)cyclohexyl]thiazol-5-yl]phenyl]carbamate

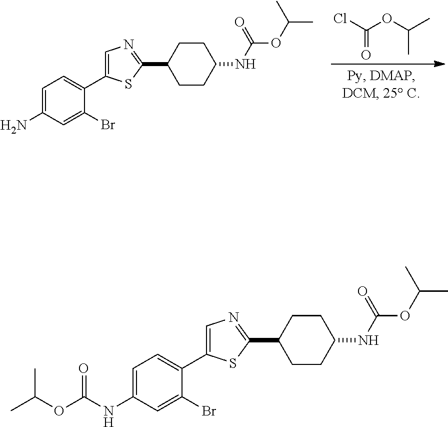

A mixture of trans-isopropyl N-[4-[5-(4-amino-2-bromo-phenyl)tiazol-2-yl]cyclohexyl]carbamate (1.0 g, 2 mmol, 1.0 eq.), isopropyl chloroformate (419 mg, 3 mmol, 1.5 eq.), Py (541 mg, 7 mmol, 3.0 eq.) and DMAP (28 mg, 228 umol, 0.1 eq.) in DCM (30 mL) was stirred at 25° C. for 1 hr under N₂ atmosphere. The mixture was quenched by H₂O (100 mL) and extracted with DCM (100 mL×3). The combined organic layers were dried over Na₂SO₄, filtered, concentrated and purified by prep-HPLC (column: Phenomenex luna C18 250×50 mm×10 um; mobile phase: [water(0.1% TFA)-ACN]; B %: 45%-75%, 20 mins) to yield trans-isopropyl N-[3-bromo-4-[2-[4-(isopropoxycarbonylamino)cyclohexyl]thiazol-5-yl]phenyl]carbamate (280 mg, 534 umol, 23.4% yield) as a gray solid. ¹H NMR (400 MHz, methanol-d4) Shift 7.94 (d, J=1.96 Hz, 1H), 7.69-7.79 (m, 1H), 7.37-7.56 (m, 2H), 4.93-5.07 (m, 1H), 4.77-4.82 (m, 1H), 3.42-3.54 (m, 1H), 2.96-3.11 (m, 1H), 2.24 (br d, J=11.98 Hz, 2H), 2.03-2.16 (m, 2H), 1.64-1.80 (m, 2H), 1.37-1.52 (m, 2H), 1.33 (d, J=6.24 Hz, 6H), 1.25 (br d, J=6.11 Hz, 6H). ESI [M+H]=526.2/524.2

### Step 8: Synthesis of isopropyl ((trans)-4-(5-(4-((isopropoxycarbonyl)amino)-2-morpholinophenyl)thiazol-2-yl)cyclohexyl)carbamate (1)

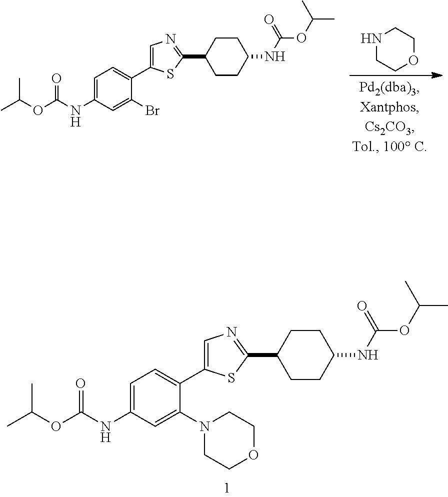

A mixture of morpholine (25 mg, 286 umol, 3.0 eq.), trans-isopropyl N-[3-bromo-4-[2-[4-(isopropoxycarbonylamino)cyclohexyl]thiazol-5-yl]phenyl]carbamate (50 mg, 95 umol, 1.0 eq.), Xantphos (11 mg, 19 umol, 0.2 eq.), Pd₂(dba)₃ (17 mg, 19 umol, 0.2 eq.) and Cs₂CO₃ (62 mg, 191 umol, 2.0 eq.) in Tol. (3 mL) was stirred at 100° C. for 12 hrs under N₂ atmosphere. The mixture was quenched by H₂O (20 mL) and extracted with EtOAc (50 mL*3). The combined organic layers were dried over Na₂SO₄, filtered, concentrated and purified by prep-HPLC (column: Welch Ultimate AQ-C18 150×30 mm×5 um; mobile phase: [water(0.1% TFA)-ACN]; B %: 45%-75%, 12 mins), (column: Waters Xbridge 150×25 5u; mobile phase: [water(10 mM NH₄HCO₃)-ACN]; B %: 50%-60%, 7 mins) to yield trans-isopropyl N-[4-[2-[4-(isopropoxycarbonylamino)cyclohexyl]thiazol-5-yl]-3-morpholinophenyl]carbamate (4 mg, 8 umol, 7.9% yield, 99.6% purity) as a pale yellow solid. ¹H NMR (400 MHz, methanol-d4) δ 7.88 (s, 1H), 7.40-7.50 (m, 2H), 7.20 (dd, J=1.87, 8.49 Hz, 1H), 4.96 (td, J=6.20, 12.51 Hz, 1H), 4.73 (s, 1H), 3.75-3.88 (m, 4H), 3.39-3.55 (m, 1H), 2.92-3.04 (m, 1H), 2.82-2.92 (m, 4H), 2.19 (br d, J=13.45 Hz, 2H), 2.07 (br d, J=10.58 Hz, 2H), 1.60-1.77 (m, 2H), 1.35-1.51 (m, 2H), 1.30 (d, J=6.39 Hz, 6H), 1.22 (br d, J=6.17 Hz, 6H). ESI [M+H]=531.3

### Example 2: Preparation of isopropyl ((trans)-4-(5-(4-((isopropoxycarbonyl)amino)-2-((methylamino)methyl)phenyl)thiazol-2-yl)cyclohexyl)carbamate

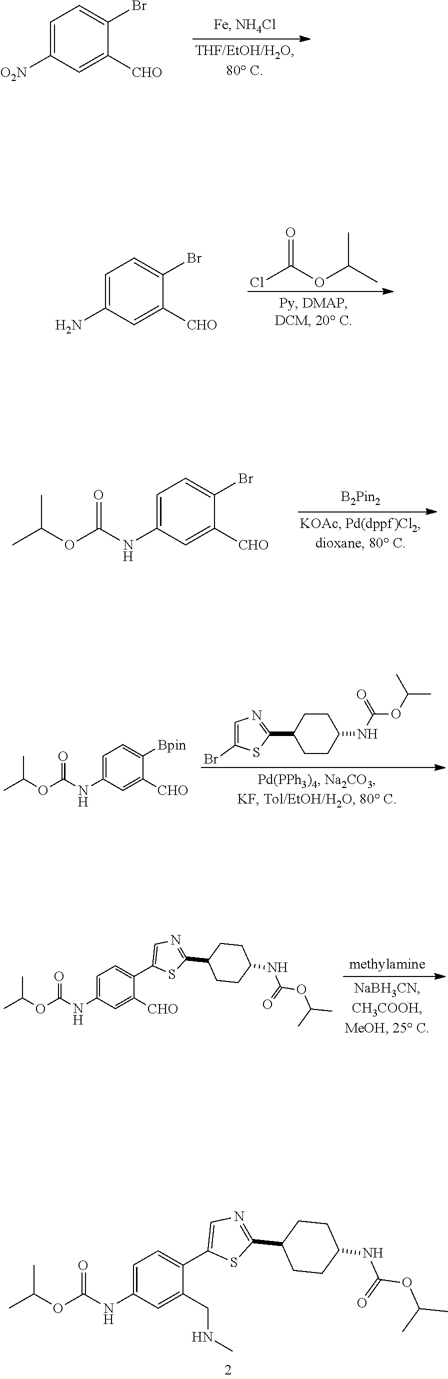

### Step 1: Synthesis of 5-amino-2-bromo-benzaldehyde

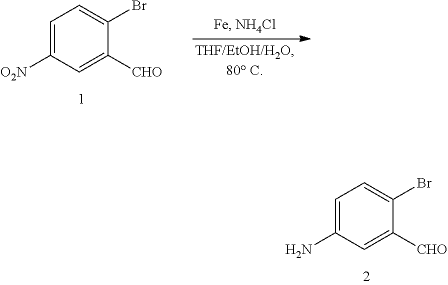 [SMILES: O=Cc1cc([N+](=O)[O-])ccc1Br ; Nc1ccc(Br)c(C=O)c1]

To a solution of 2-bromo-5-nitro-benzaldehyde (3.0 g, 13 mmol, 1.0 eq.) in THF (30 mL)/EtOH (30 mL)/H₂O (10 mL) was added Fe (3.6 g, 65 mmol, 5.0 eq.) and NH₄Cl (2.1 g, 39 mmol, 3.0 eq.). The mixture was stirred at 80° C. for 2 hrs, filtered, concentrated, diluted with H₂O (100 mL) and extracted with EtOAc (300 mL×3). The combined organic layers were dried over Na₂SO₄ and concentrated to yield 5-amino-2-bromo-benzaldehyde (2.6 g, crude) as a yellow solid.

### Step 2: Synthesis of isopropyl N-(4-bromo-3-formyl-phenyl)carbamate

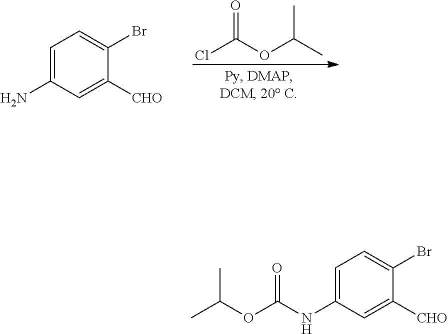

To a solution of 5-amino-2-bromo-benzaldehyde (2.6 g, 13 mmol, 1.0 eq.) in CH₂Cl₂ (25 mL) was added isopropyl chloroformate (1.6 g, 13 mmol, 1.0 eq.), pyridine (1.0 g, 13 mmol, 1.0 eq.) and DMAP (1.6 g, 13 mmol, 1.0 eq.). The mixture was stirred at 20° C. for 12 hrs, quenched by H₂O (100 mL) and extracted with EtOAc (500 mL×3). The combined organic layers were dried over Na₂SO₄, filtered, concentrated and purified by column chromatography (SiO₂, Petroleum ether:EtOAc=20:1 to 1:1) to yield isopropyl N-(4-bromo-3-formyl-phenyl)carbamate (1.0 g, 3.5 mmol, 26.9% yield) as a white solid. ¹H NMR (400 MHz, methanol-d4) δ=10.25-10.19 (m, 1H), 7.93 (d, J=2.6 Hz, 1H), 7.67 (d, J=2.6 Hz, 1H), 7.58-7.54 (m, 1H), 4.91-4.88 (m, 1H), 1.24 (d, J=2.9 Hz, 6H).

### Step 3: Synthesis of isopropyl (3-formyl-4-(4,4,5,5-tetramethyl-1,3,2-dioxaborolan-2-yl)phenyl)carbamate

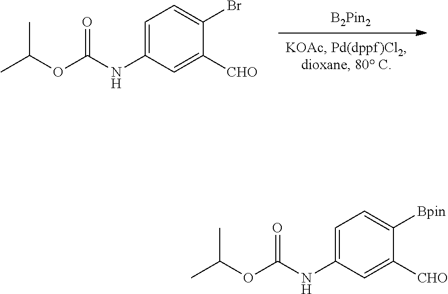

To a solution of isopropyl N-(4-bromo-3-formyl-phenyl)carbamate (260 mg, 909 umol, 1.0 eq.) in dioxane (20 mL) was added 4,4,5,5-tetramethyl-2-(4,4,5,5-tetramethyl-1,3,2-dioxaborolan-2-yl)-1,3,2-dioxaborolane (277 mg, 1 mmol, 1.2 eq.), KOAc (268 mg, 3 mmol, 3.0 eq.) and Pd(dppf)Cl₂ (66 mg, 91 umol, 0.1 eq.). The mixture was stirred at 80° C. for 12 hrs and concentrated to yield isopropyl (3-formyl-4-(4,4,5,5-tetramethyl-1,3,2-dioxaborolan-2-yl)phenyl)carbamate (300 mg, crude) as a yellow solid.

### Step 4: Synthesis of isopropyl trans-N-[3-formyl-4-[2-[4-(isopropoxy-carbonylamino)cyclohexyl]thiazol-5-yl]phenyl]carbamate

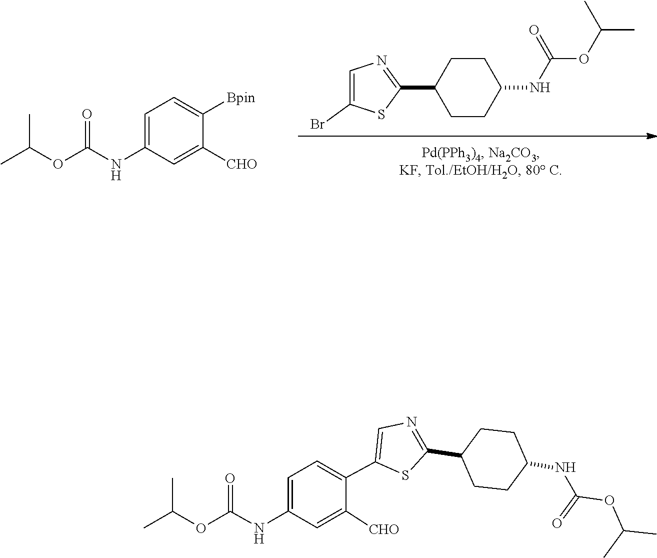

To a solution of isopropyl trans-N-[4-(5-bromothiazol-2-yl)cyclohexyl]carbamate (300 mg, 864 umol, 1.0 eq.) in EtOH (3 mL)/toluene (3 mL)/H₂O (1 mL) was added isopropyl N-[3-formyl-4-(4,4,5,5-tetramethyl-1,3,2-dioxaborolan-2-yl)phenyl]carbamate (288 mg, 864 umol, 1.0 eq.), KF (75 mg, 1 mmol, 1.5 eq.), Na₂CO₃ (183 mg, 2 mmol, 2.0 eq.), Pd(PPh₃)₄ (100 mg, 86 umol, 0.1 eq.) and stirred at 80° C. for 3 hrs. The reaction mixture was quenched by H₂O (30 mL) and then extracted with EtOAc (60 mL×3). The combined organic layers were dried over Na₂SO₄, filtered, concentrated and purified by column chromatography (SiO₂, Petroleum ether:EtOAc=3:1 to 1:1) to yield isopropyl trans-N-[3-formyl-4-[2-[4-(isopropoxycarbonylamino)cyclohexyl]thiazol-5-yl]phenyl]carbamate (200 mg, 422 umol, 48.9% yield) as a yellow solid. ESI [M+H]=474.3

### Step 5: Synthesis of isopropyl ((trans)-4-(5-(4-((isopropoxycarbonyl)amino)-2-((methylamino)methyl)phenyl)thiazol-2-yl)cyclohexyl)carbamate (2)

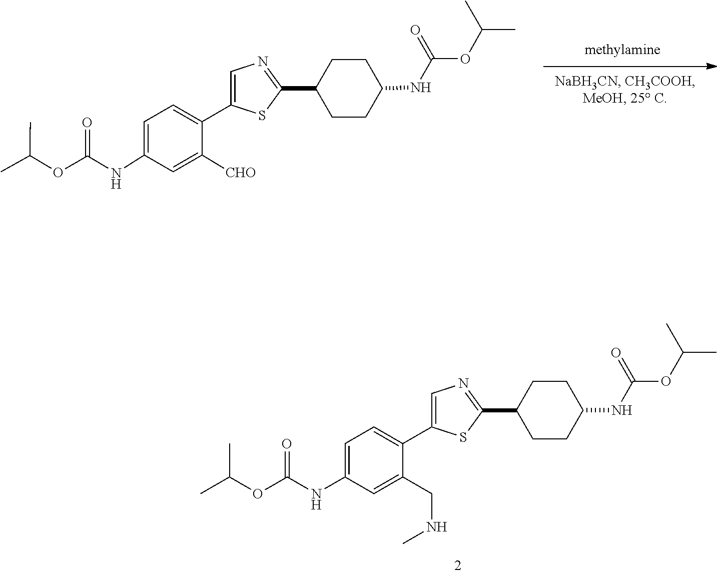

To a solution of methylamine (9 mg, 285 umol, 1.5 eq.) in MeOH (2 mL) was added NaBH₃CN (36 mg, 570 umol, 3.0 eq.), CH₃COOH (1 mg, 19 umol, 0.1 eq.) and isopropyl trans-N-[3-formyl-4-[2-[4-(isopropoxycarbonylamino)cyclohexyl]thiazol-5-yl]phenyl]carbamate (90 mg, 190 umol, 1.0 eq.). The mixture was stirred at 25° C. for 1 hr, quenched by sat. aq. NaHCO₃ (10 mL), and extracted with EtOAc (20 mL×3). The combined organic layers were dried over Na₂SO₄, filtered, concentrated and purified by prep-HPLC (column: Nano-Micro UniSil 5-100 C18 ULTRA 100×250 mm Sum; mobile phase: [water(TFA)-ACN]; B %: 28%-45%, 10 mins) to yield isopropyl ((trans)-4-(5-(4-((isopropoxycarbonyl)amino)-2-((methylamino)methyl)phenyl) thiazol-2-yl)cyclohexyl)carbamate (2) (23 mg, 43 umol, 22.4% yield, 91.5% purity) as a yellow solid ¹H NMR (400 MHz, methanol-d4) δ=7.92 (br s, 1H), 7.64 (s, 1H), 7.43 (s, 2H), 5.09-4.93 (m, 1H), 4.87-4.78 (m, 1H), 4.29 (s, 2H), 3.53-3.41 (m, 1H), 3.10-2.99 (m, 1H), 2.70 (s, 3H), 2.25 (br d, J=12.5 Hz, 2H), 2.09 (br d, J=10.1 Hz, 2H), 1.72 (dq, J=2.8, 12.7 Hz, 2H), 1.52-1.38 (m, 2H), 1.34 (d, J=6.2 Hz, 6H), 1.25 (br d, J=6.2 Hz, 6H). ESI [M+H]=489.2

### Example 3: Preparation of isopropyl ((tran)-4-(5-(2-acetamido-4-((isopropoxy-carbonyl)amino)phenyl)thiazol-2-yl)cyclohexyl)carbamate

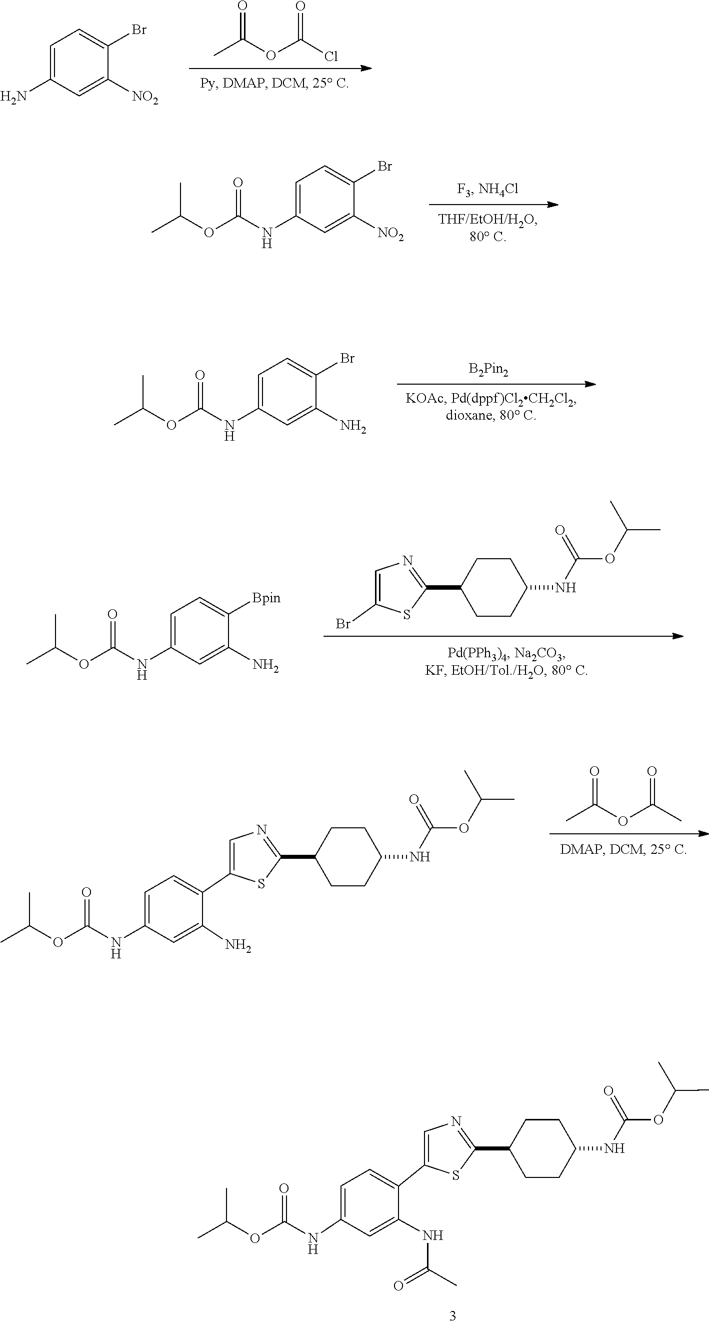

### Step 1: Synthesis of isopropyl N-(4-bromo-3-nitro-phenyl)carbamate

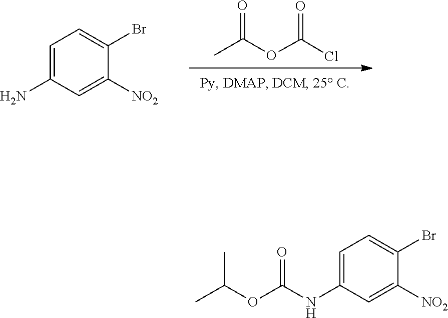

To a solution of isopropyl chloroformate (3.3 g, 27 mmol, 1.5 eq.) in DCM (30 mL) was added pyridine (4.3 g, 55 mmol, 3.0 eq.), 4-bromo-3-nitro-aniline (4.0 g, 18 mmol, 1.0 eq.) and DMAP (225 mg, 1.8 mmol, 0.1 eq.). The mixture was stirred at 25° C. for 0.2 hr, quenched by HCl (10 mL) at 25° C. and extracted with DCM (80 mL×3). The combined organic layers were washed with sat. aq. Na₂CO₃ (20 mL×3), dried over Na₂SO₄, filtered, concentrated and purified by column chromatography (SiO₂, Petroleum ether:EtOAc=1:0 to 10:1) to yield isopropyl N-(4-bromo-3-nitro-phenyl)carbamate (4.5 g, 14 mmol, 80.5% yield) as a yellow solid.

### Step 2: Synthesis of isopropyl N-(3-amino-4-bromo-phenyl)carbamate

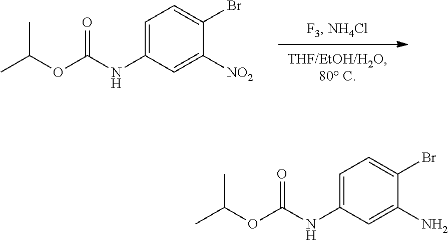 [SMILES: CC(C)OC(=O)Nc1ccc(Br)c([N+](=O)[O-])c1 ; CC(C)OC(=O)Nc1ccc(Br)c(N)c1]

A mixture of isopropyl N-(4-bromo-3-nitro-phenyl)carbamate (4.4 g, 14 mmol, 1.0 eq.), Fe (4.1 g, 73 mmol, 5.0 eq.) and NH₄Cl (2.3 g, 44 mmol, 3.0 eq.) in EtOH (9 mL)/THF (9 mL)/H₂O (3 mL) was stirred at 80° C. for 12 hrs under N₂ atmosphere. The mixture was filtered, concentrated, diluted with H₂O (30 mL) and extracted with EtOAc (60 mL×3). The combined organic layers were dried over Na₂SO₄, filtered, concentrated and purified by column chromatography (SiO₂, Petroleum ether:EtOAc=1:0 to 3:1) to yield isopropyl N-(3-amino-4-bromo-phenyl)carbamate (3.5 g, 12 mmol, 86.7% yield) as a pale yellow solid. ESI [M+H]=273.1/275.1

### Step 3: Synthesis of isopropyl N-[3-amino-4-(4,4,5,5-tetramethyl-1,3,2-dioxaborolan-2-yl)phenyl]carbamate

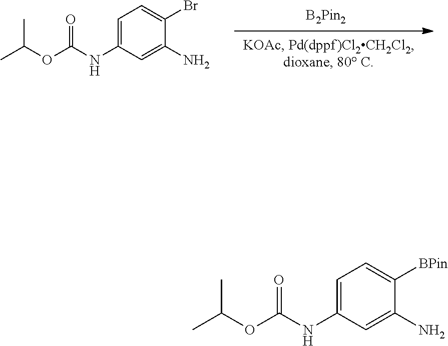

A mixture of 4,4,5,5-tetramethyl-2-(4,4,5,5-tetramethyl-1,3,2-dioxaborolan-2-yl)-1,3,2-dioxaborolane (9.7 g, 38 mmol, 3.0 eq.), isopropyl N-(3-amino-4-bromo-phenyl)carbamate (3.5 g, 12 mmol, 1.0 eq.), KOAc (3.7 g, 38 mmol, 3.0 eq.) and Pd(dppf)Cl₂·CH₂Cl₂ (1.0 g, 1 mmol, 0.1 eq.) in dioxane (30 mL) was stirred at 80° C. for 12 hrs under N₂ atmosphere. The mixture was concentrated and purified by column chromatography (SiO₂, Petroleum ether:EtOAc=40:1 to 5:1) to yield isopropyl N-[3-amino-4-(4,4,5,5-tetramethyl-1,3,2-dioxaborolan-2-yl)phenyl]carbamate (0.9 g, 2 mmol, 21.9% yield) as a white solid. ESI [M+H]=321.2

### Step 4: Synthesis of isopropyl trans-N-[3-amino-4-[2-[4-(isopropoxy-carbonylamino)cyclohexyl]thiazol-5-yl]phenyl]carbamate

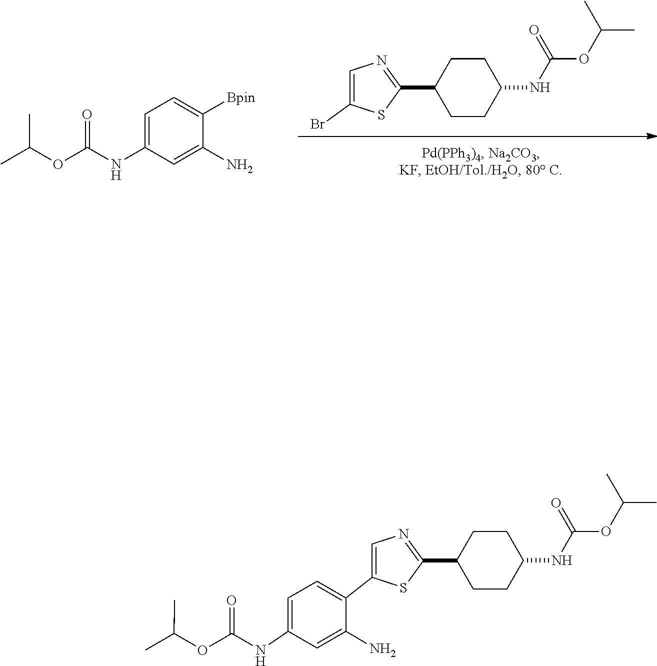

A mixture of isopropyl trans-N-[4-(5-bromothiazol-2-yl)cyclohexyl]carbamate (295 mg, 851 umol, 1.0 eq.), isopropyl N-[3-amino-4-(4,4,5,5-tetramethyl-1,3,2-dioxaborolan-2-yl)phenyl]carbamate (300 mg, 936 umol, 1.1 eq.), Na₂CO₃ (270 mg, 2 mmol, 3.0 eq.), KF (74 mg, 1 mmol, 1.5 eq.) and Pd(PPh₃)₄ (98 mg, 85 umol, 0.1 eq.) in toluene (3 mL)/EtOH (3 mL)/H₂O (1 mL) was stirred at 80° C. for 12 hrs under N₂ atmosphere. The reaction mixture was quenched by H₂O (20 mL) and extracted with EtOAc (50 mL×2). The combined organic layers were dried over Na₂SO₄, filtered, concentrated and purified by column chromatography (SiO₂, Petroleum ether:EtOAc=20:1 to 0:1) to yield isopropyl trans-N-[3-amino-4-[2-[4-(isopropoxy-carbonylamino)cyclohexyl]thiazol-5-yl]phenyl]carbamate (400 mg, crude) as a yellow solid. ESI [M+H]=461.3

### Step 5: Synthesis of isopropyl ((tran)-4-(5-(2-acetamido-4-((isopropoxy-carbonyl)amino)phenyl)thiazol-2-yl)cyclohexyl)carbamate (3)

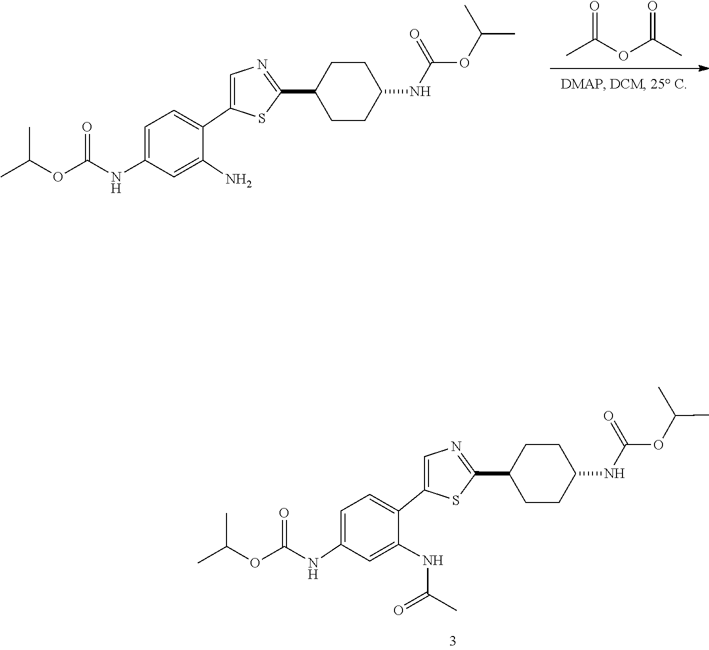

A mixture of acetyl acetate (6 mg, 65 umol, 1.5 eq.), isopropyl trans-N-[3-amino-4-[2-[4-(isopropoxycarbonylamino)cyclohexyl]thiazol-5-yl]phenyl]carbamate (30 mg, 65 umol, 1.5 eq.), DMAP (530 ug, 4 umol, 0.1 eq.) in DCM (5 mL) was stirred at 25° C. for 2 hrs under N₂ atmosphere. The reaction mixture was quenched by H₂O (5 mL) and extracted with DCM (30 mL×2). The combined organic layers were dried over Na₂SO₄, filtered, concentrated and purified by prep-HPLC (column: Waters Xbridge 150*25 5u; mobile phase: [water(10 mM NH₄HCO₃)-ACN]; B %: 30%-60%, 7 mins) to yield isopropyl ((tran)-4-(5-(2-acetamido-4-((isopropoxy-carbonyl)amino)phenyl) thiazol-2-yl)cyclohexyl)carbamate (3) (3 mg, 6 umol, 15.5% yield) as a pale yellow solid ¹H NMR (400 MHz, methanol-d4) δ 7.73 (s, 1H), 7.60 (d, J=1.59 Hz, 1H), 7.41-7.52 (m, 2H), 4.93-5.03 (m, 1H), 4.78-4.87 (m, 1H), 3.43-3.53 (m, 1H), 3.00 (tt, J=12.13, 3.45 Hz, 1H), 2.22 (br d, J=12.35 Hz, 2H), 2.11 (s, 3H), 2.03-2.10 (m, 2H), 1.64-1.76 (m, 2H), 1.39-1.48 (m, 2H), 1.32 (d, J=6.24 Hz, 6H), 1.24 (br d, J=6.11 Hz, 6H) ESI [M+H]=503.2

### Example 4: Preparation of isopropyl ((trans)-4-(5-(4-((isopropoxycarbonyl)amino)-2-ureidophenyl)thiazol-2-yl)cyclohexyl)carbamate

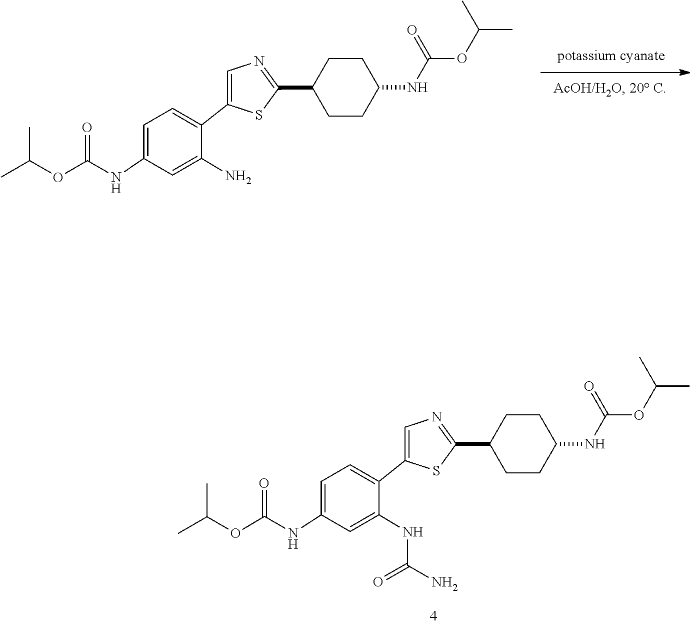

A mixture of isopropyl trans-N-[3-amino-4-[2-[4-(isopropoxycarbonylamino)cyclohexyl]thiazol-5-yl]phenyl]carbamate (30 mg, 65 umol, 1.0 eq.), potassium cyanate (10 mg, 130 umol, 2.0 eq.) in AcOH (3 mL)/H₂O (2 mL) was stirred at 20° C. for 6 hrs under N₂ atmosphere, quenched by H₂O (10 mL) and extracted with EtOAc (20 mL×3). The combined organic layers were dried over Na₂SO₄, filtered, concentrated and purified by prep-HPLC (column: Welch Ultimate AQ-C18 150*30 mm×5 um; mobile phase: [water(0.1% TFA)-ACN]; B %: 35%-65%, 12 mins) to yield isopropyl ((trans)-4-(5-(4-((isopropoxycarbonyl)amino)-2-ureidophenyl) thiazol-2-yl)cyclohexyl)carbamate (4) (14 mg, 27 umol, 42.6% yield) as a white solid.

¹H NMR (400 MHz, metahnol-d4) δ=7.79 (s, 1H), 7.65 (d, J=1.76 Hz, 1H), 7.34-7.45 (m, 2H), 4.95 (td, J=6.20, 12.51 Hz, 1H), 4.82 (br d, J=5.95 Hz, 1H), 3.44 (br t, J=11.47 Hz, 1H), 2.94-3.08 (m, 1H), 2.21 (br d, J=12.79 Hz, 2H), 2.06 (br d, J=10.58 Hz, 2H), 1.62-1.76 (m, 2H), 1.34-1.48 (m, 2H), 1.29 (d, J=6.17 Hz, 6H), 1.15-1.25 (m, 6H). ESI [M+H]=504.2

### Example 5: Preparation of isopropyl ((trans)-4-(5-(4-((isopropoxycarbonyl)amino)-2-(piperidin-1-ylmethyl)phenyl)thiazol-2-yl)cyclohexyl)carbamate

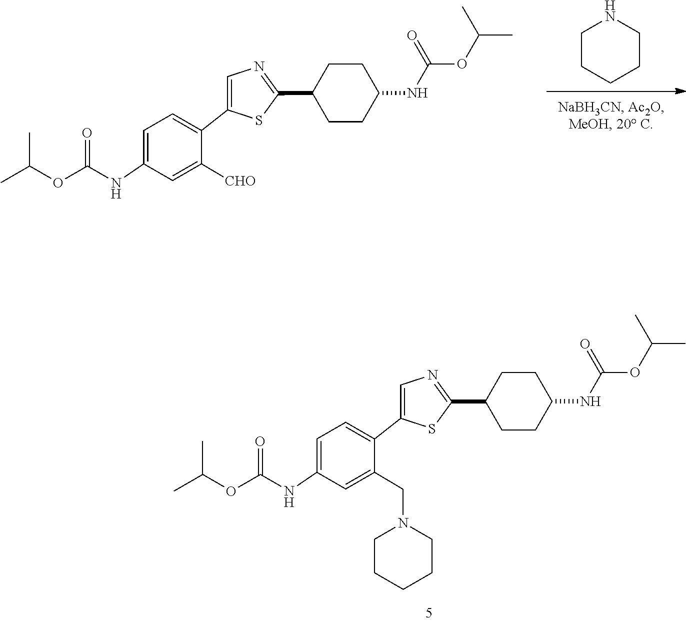

Following the same protocol and under the same conditions as for example 2, step 5, using piperidine, isopropyl ((trans)-4-(5-(4-((isopropoxycarbonyl)amino)-2-(piperidin-1-ylmethyl)phenyl) thiazol-2-yl)cyclohexyl)carbamate (5) was obtained (20 mg, 30 umol, 71.3% yield, 98.8% purity, TFA) as a white solid. ¹H NMR (400 MHz, methanol-d4) δ=7.97 (s, 1H), 7.68-7.58 (m, 1H), 7.48-7.37 (m, 2H), 5.09-4.94 (m, 1H), 4.85-4.79 (m, 1H), 4.39 (s, 2H), 3.45 (tt, J=3.9, 11.5 Hz, 1H), 3.37 (br d, J=11.9 Hz, 2H), 3.03 (tt, J=3.4, 12.0 Hz, 1H), 2.83 (br t, J=11.9 Hz, 2H), 2.29-2.20 (m, 2H), 2.12-2.01 (m, 2H), 1.90-1.80 (m, 2H), 1.77-1.64 (m, 4H), 1.51-1.35 (m, 4H), 1.34-1.29 (m, 6H), 1.26-1.20 (m, 6H). ESI [M+H]=543.3

### Example 6: Preparation of isopropyl ((trans)-4-(5-(2-((dimethylamino)methyl)-4-((isopropoxycarbonyl)amino)phenyl)thiazol-2-yl)cyclohexyl)carbamate

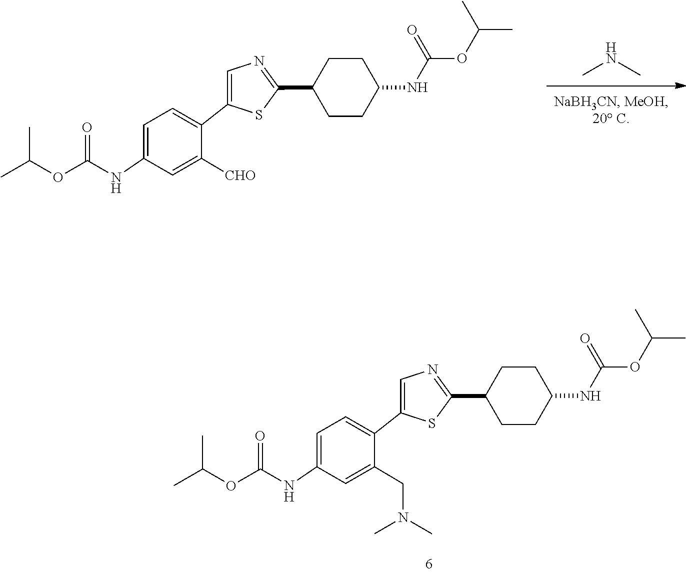

Following the same protocol and under the same conditions as for example 2, step 5, using dimethylamine, isopropyl ((trans)-4-(5-(2-((dimethylamino)methyl)-4-((isopropoxy-carbonyl)amino)phenyl)thiazol-2-yl)cyclohexyl)carbamate was obtained (13 mg, 21 umol, 49.9% yield, 100.0% purity, TFA) as a white solid. ¹H NMR (400 MHz, methanl-d4) δ=7.97-7.89 (m, 1H), 7.67-7.60 (m, 1H), 7.49-7.39 (m, 2H), 5.03-4.94 (m, 1H), 4.82 (br s, 1H), 4.42 (s, 2H), 3.50-3.39 (m, 1H), 3.08-2.95 (m, 1H), 2.78 (s, 6H), 2.29-2.20 (m, 2H), 2.11-2.03 (m, 2H), 1.70 (dq, J=2.9, 12.9 Hz, 2H), 1.48-1.35 (m, 2H), 1.32 (d, J=6.2 Hz, 6H), 1.22 (br d, J=6.2 Hz, 6H). ESI [M+H]=503.3

### Example 7: Preparation of isopropyl ((trans)-4-(5-(4-((isopropoxycarbonyl)amino)-2-(morpholinomethyl)phenyl)thiazol-2-yl)cyclohexyl)carbamate

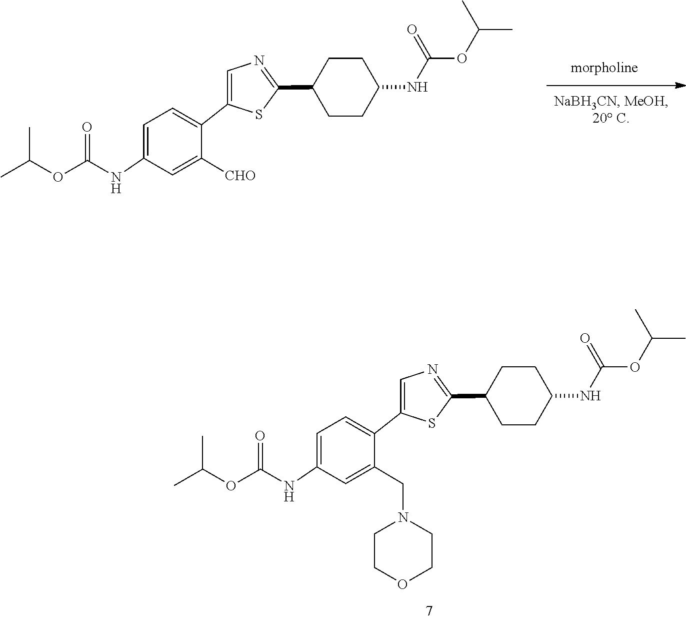

Following the same protocol and under the same conditions as for example 2, step 5, using piperidine, isopropyl ((trans)-4-(5-(4-((isopropoxycarbonyl)amino)-2-(morpholino-methyl)phenyl)thiazol-2-yl)cyclohexyl)carbamate was obtained (20 mg, 31 umol, 48.6% yield, TFA) as a pale yellow solid. ¹H NMR (400 MHz, methanol-d4) δ=8.04 (s, 1H), 7.66 (s, 1H), 7.41-7.49 (m, 2H), 4.42-4.56 (m, 1H), 3.83-4.00 (m, 1H), 3.47 (tt, J=11.63, 3.84 Hz, 2H), 3.11-3.28 (m, 4H), 3.45-3.51 (m, 1H), 2.97-3.30 (m, 5H), 2.06-2.15 (m, 2H), 2.19-2.33 (m, 2H), 1.73 (qd, J=12.82, 3.00 Hz, 2H), 1.40-1.48 (m, 2H), 1.34 (d, J=6.24 Hz, 6H), 1.25 (d, J=6.11 Hz, 6H). ESI [M+H]=545.4/546.4

### Example 8: Preparation of isopropyl ((trans)-4-(5-(4-((isopropoxycarbonyl)amino)-2-(3-methylureido)phenyl)thiazol-2-yl)cyclohexyl)carbamate

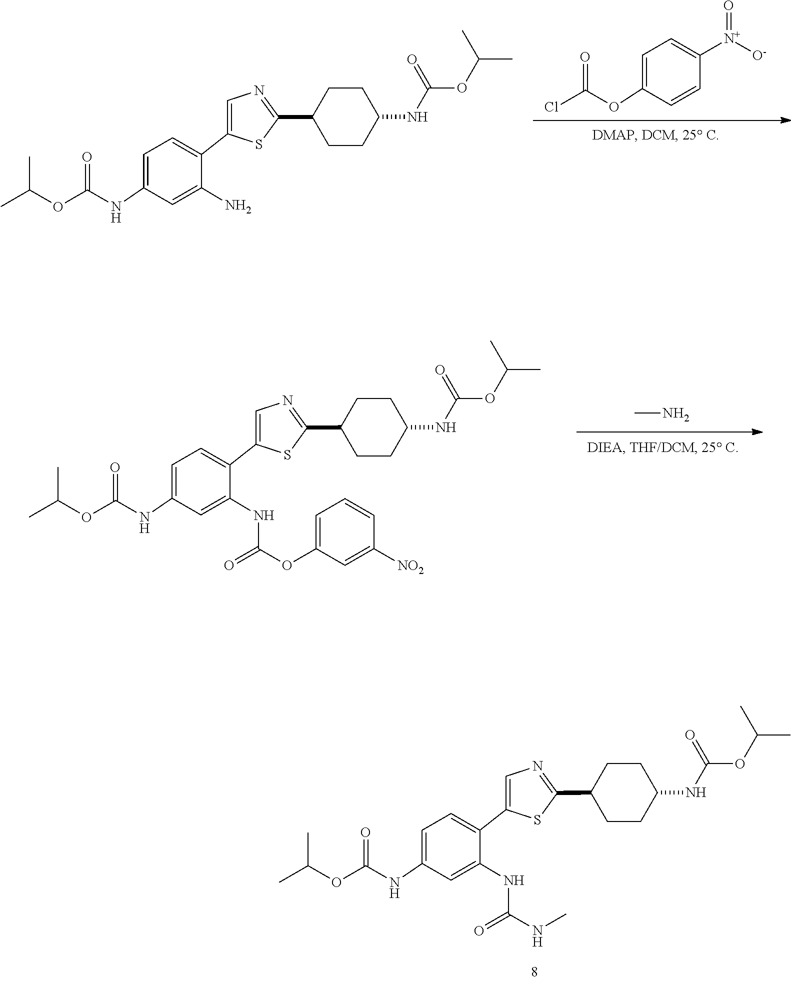

### Step 1: Synthesis of isopropyl trans-N-[4-[2-[4-(isopropoxycarbonylamino)cyclohexyl]thiazol-5-yl]-3-[(4-nitrophenoxy)carbonylamino]phenyl]carbamate

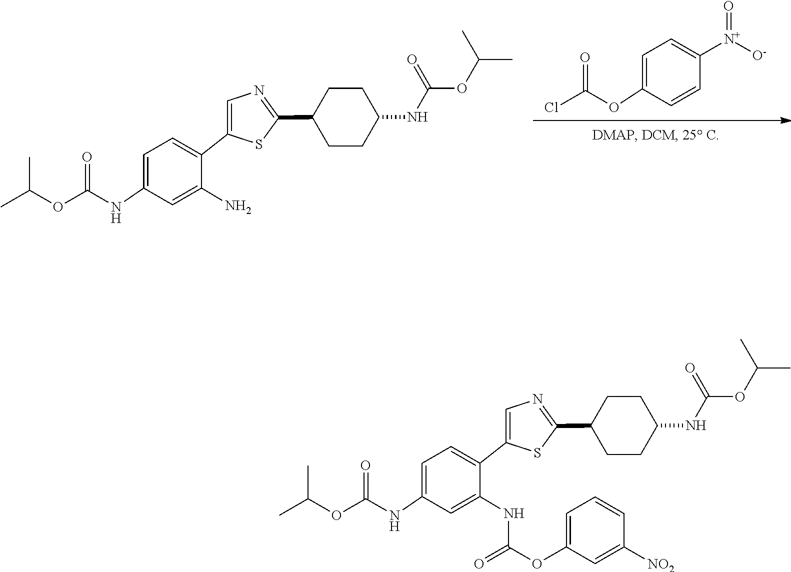

To a solution of isopropyl trans-N-[3-amino-4-[2-[4-(isopropoxycarbonyl amino)cyclohexyl]thiazol-5-yl]phenyl]carbamate (40 mg, 86 umol, 1.0 eq.) in DCM (6 mL) was added DMAP (1 mg, 8 umol, 0.1 eq.) and 4-nitrophenylchloroformate (40 mg, 199 umol, 2.3 eq.). The mixture was stirred at 25° C. for 1 hr to yield isopropyl trans-N-[4-[2-[4-(isopropoxycarbonylamino)cyclohexyl]thiazol-5-yl]-3-[(4-nitrophenoxy)carbonylamino]phenyl]carbamate (54 mg, crude) as a yellow solution.

ESI [M+H]=626.3

### Step 2: Synthesis of isopropyl ((trans)-4-(5-(4-((isopropoxycarbonyl)amino)-2-(3-methylureido)phenyl)thiazol-2-yl)cyclohexyl)carbamate (8)

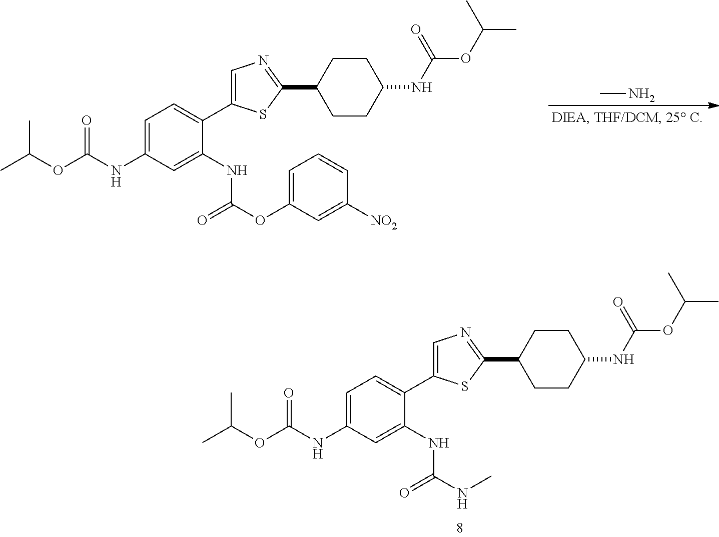

To a solution of methylamine (4 mg, 129 umol, 3.0 eq.) in THF (5 mL) was added a solution of DIEA (5 mg, 43 umol, 1.0 eq.) and isopropyl trans-N-[4-[2-[4-(isopropoxycarbonylamino)cyclohexyl]thiazol-5-yl]-3-[(4-nitrophenoxy)carbonyl amino]phenyl]carbamate (27 mg, 43 umol, 1.0 eq.) in DCM (3 mL). The mixture was stirred at 25° C. for 1 hr, quenched by HCl (2 mL) and extracted with DCM (10 mL×2). The combined organic layers were dried over Na₂SO₄, filtered, concentrated and purified by prep-HPLC (column: Nano-Micro UniSil 5-100 C18 ULTRA100*250 mm*5 um; mobile phase: [water(0.1% TFA)-ACN]; B %: 42%-52%, 10 mins) to yield isopropyl ((trans)-4-(5-(4-((isopropoxycarbonyl)amino)-2-(3-methylureido)phenyl)thiazol-2-yl)cyclohexyl)carbamate (5 mg, 10 umol, 24.2% yield) as a white solid. ¹H NMR (400 MHz, methanol-d4) δ=7.73-7.81 (m, 1H), 7.68 (d, J=1.96 Hz, 1H), 7.33-7.45 (m, 2H), 4.78-4.85 (m, 1H), 3.44-3.57 (m, 1H), 3.43-3.50 (m, 1H), 2.95-3.05 (m, 1H), 2.70 (s, 3H), 2.18-2.27 (m, 2H), 2.04-2.14 (m, 2H), 1.63-1.77 (m, 2H), 1.36-1.48 (m, 2H), 1.32 (d, J=6.24 Hz, 6H), 1.21-1.28 (m, 6H). ESI [M+H]=518.2

### Example 9: Preparation of isopropyl ((trans)-4-(5-(2-((2-(dimethylamino)ethyl)(methyl)amino)-4-((isopropoxycarbonyl)amino)phenyl)thiazol-2-yl)cyclohexyl)carbamate

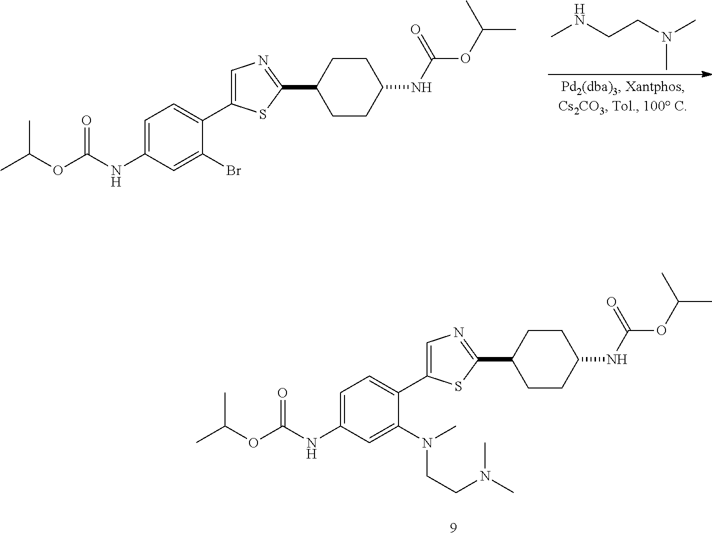

Following the same protocol and under the same conditions as for example 1, step 7, using N,N′,N′-trimethylethane-1,2-diamine, isopropyl ((trans)-4-(5-(2-((2-(dimethylamino)ethyl)(methyl)amino)-4-((isopropoxycarbonyl)amino)phenyl)thiazol-2-yl)cyclohexyl) carbamate (9) was obtained (16 mg, 24 umol, 25.3% yield, 99.5% purity, TFA) as a yellow solid. ¹H NMR (400 MHz, methanol-d4) δ=8.14 (s, 1H), 7.75-7.59 (m, 2H), 7.21 (dd, J=2.1, 8.6 Hz, 1H), 5.03-4.96 (m, 1H), 4.85 (br d, J=6.1 Hz, 1H), 3.48 (tt, J=3.7, 11.6 Hz, 1H), 3.42-3.36 (m, 2H), 3.31-3.25 (m, 2H), 3.16-3.04 (m, 1H), 2.89 (s, 6H), 2.69 (s, 3H), 2.24 (br d, J=12.3 Hz, 2H), 2.10 (br d, J=10.4 Hz, 2H), 1.74 (dq, J=2.9, 12.8 Hz, 2H), 1.44 (dq, J=3.3, 12.6 Hz, 2H), 1.33 (d, J=6.2 Hz, 6H), 1.25 (d, J=6.1 Hz, 6H). ESI [M+H]=546.3

### Example 10: Preparation of isopropyl ((trans)-4-(5-(4-((isopropoxy carbonyl)amino)-2-propionamidophenyl)thiazol-2-yl)cyclohexyl)carbamate

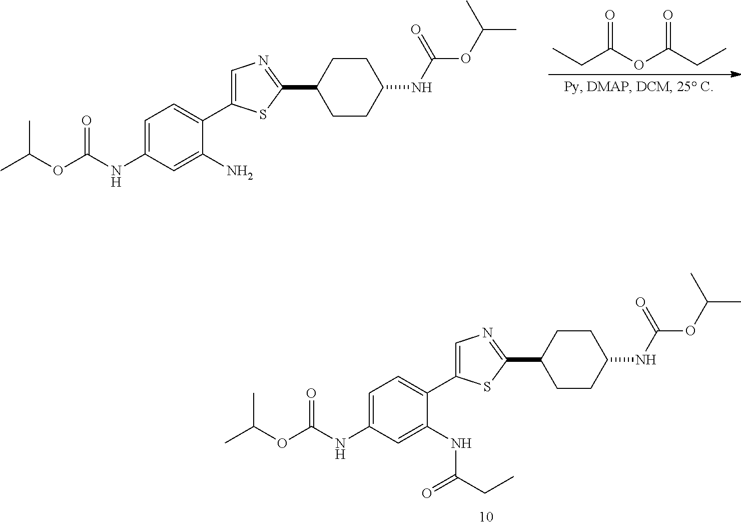

Following the same protocol and under the same conditions as for example 3, step 5, using propionic anhydride, isopropyl ((trans)-4-(5-(4-((isopropoxycarbonyl)amino)-2-propionamidophenyl)thiazol-2-yl)cyclohexyl)carbamate was obtained (7 mg, 13 umol, 62.4% yield, 100.0% purity) as a pale yellow solid. ¹H NMR (400 MHz, methanol-d4) δ=7.70 (s, 1H), 7.57 (s, 1H), 7.37-7.51 (m, 2H), 4.92-5.04 (m, 1H), 4.83 (br d, J=6.58 Hz, 1H), 3.39-3.52 (m, 1H), 2.97 (br t, J=12.06 Hz, 1H), 2.36 (q, J=7.60 Hz, 2H), 2.18 (br d, J=13.15 Hz, 2H), 2.05 (br d, J=10.52 Hz, 2H), 1.59-1.73 (m, 2H), 1.34-1.47 (m, 2H), 1.30 (d, J=6.58 Hz, 6H), 1.12-1.26 (m, 9H). ESI [M+H]=517.2

### Example 11: Preparation of isopropyl ((trans)-4-(5-(4-((isopropoxycarbonyl)amino)-2-(2,2,2-trifluoroacetamido)phenyl)thiazol-2-yl)cyclohexyl)carbamate

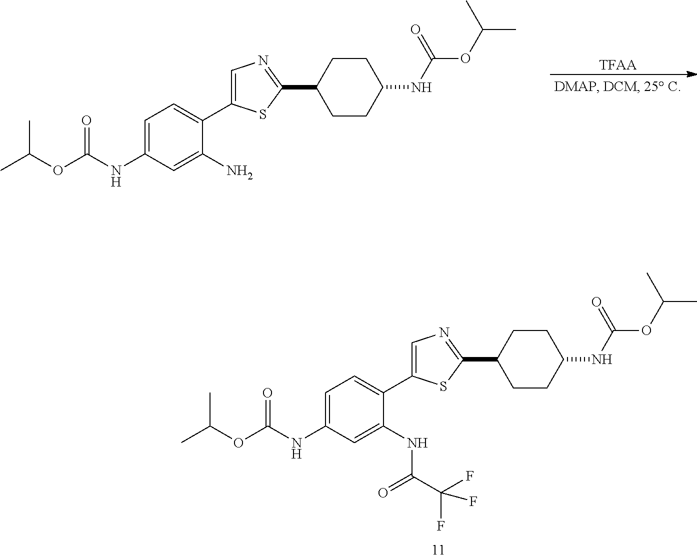

Following the same protocol and under the same conditions as for example 3, step 5, using trifluoroacetic anhydride, isopropyl ((trans)-4-(5-(4-((isopropoxycarbonyl)amino)-2-(2,2,2-trifluoroacetamido)phenyl)thiazol-2-yl)cyclohexyl)carbamate was obtained (13 mg, 24 umol, 37.9% yield) as a pale yellow solid. ¹H NMR (400 MHz, methanol-d4) δ=7.71 (s, 1H), 7.63 (d, J=1.47 Hz, 1H), 7.49-7.56 (m, 2H), 4.95-5.03 (m, 1H), 4.86 (br s, 1H), 3.46 (s, 1H), 3.00 (tt, J=12.00, 3.47 Hz, 1H), 2.15-2.25 (m, 2H), 2.03-2.10 (m, 2H), 1.67 (qd, J=12.84, 2.93 Hz, 2H), 1.38-1.50 (m, 2H), 1.33 (d, J=6.24 Hz, 6H), 1.24 (br d, J=6.11 Hz, 6H). ESI [M+H]=557.2

### Example 12: Preparation of isopropyl ((trans)-4-(5-(2-(3-ethylureido)-4-((isopropoxycarbonyl)amino)phenyl)thiazol-2-yl)cyclohexyl)carbamate

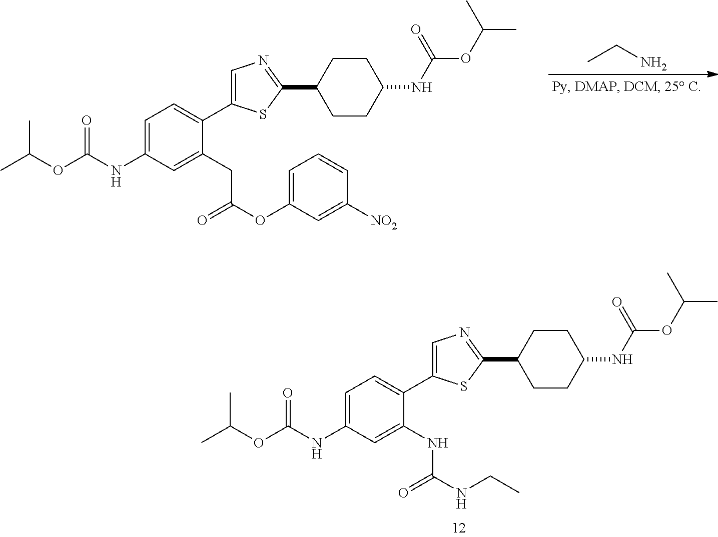

Following the same protocol and under the same conditions as for example 8, step 2, using ethylamine, isopropyl ((trans)-4-(5-(2-(3-ethylureido)-4-((isopropoxycarbonyl)amino)phenyl)thiazol-2-yl)cyclohexyl)carbamate was obtained (2 mg, 4 umol, 10.2% yield, 99.0% purity) as a yellow solid. ¹H NMR (400 MHz, methanol-d4) δ=7.73 (s, 1H), 7.68 (d, J=2.0 Hz, 1H), 7.46-7.33 (m, 2H), 4.97 (td, J=6.2, 12.5 Hz, 1H), 4.83 (br s, 1H), 3.55-3.40 (m, 1H), 3.25-3.10 (m, 2H), 3.07-2.92 (m, 1H), 2.22 (br d, J=12.8 Hz, 2H), 2.08 (br d, J=10.1 Hz, 2H), 1.77-1.63 (m, 2H), 1.42 (dq, J=3.1, 12.6 Hz, 2H), 1.32 (d, J=6.4 Hz, 6H), 1.24 (br d, J=6.1 Hz, 6H), 1.11 (t, J=7.2 Hz, 3H). ESI [M+H]=532.3

### Example 13: Preparation of isopropyl ((trans)-4-(5-(4-((isopropoxy carbonyl)amino)-2-(2-methoxyethoxy)phenyl)thiazol-2-yl)cyclohexyl)carbamate

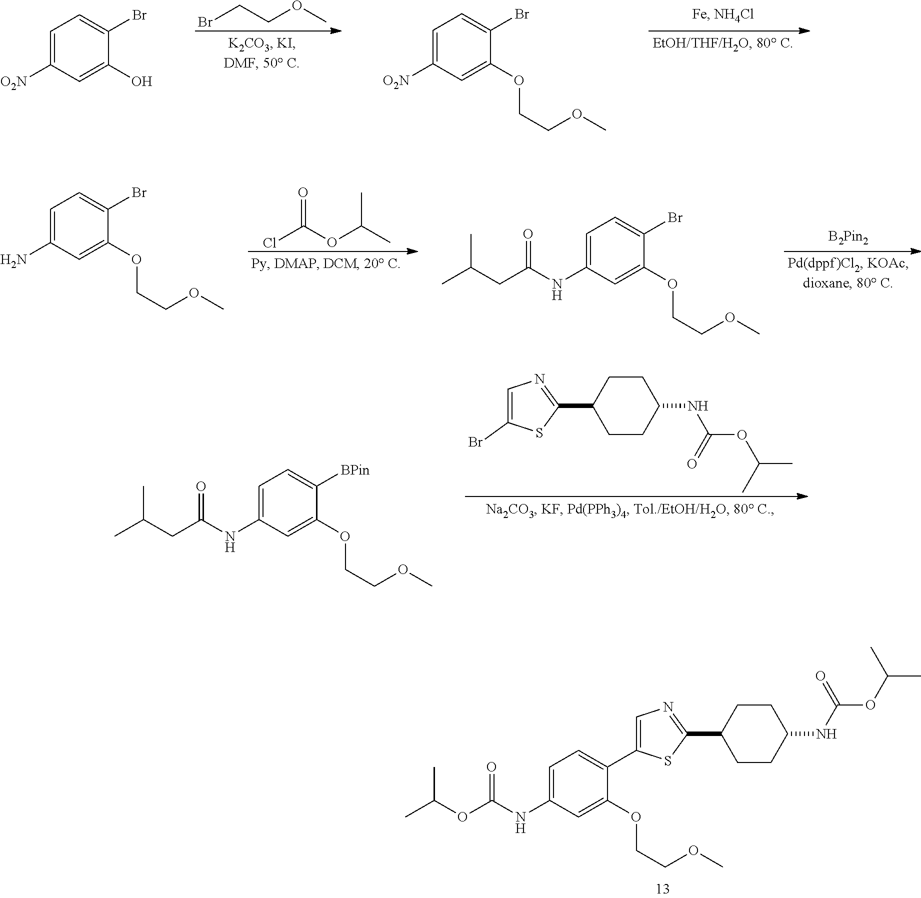

### Step 1: Synthesis of 1-bromo-2-(2-methoxyethoxy)-4-nitrobenzene

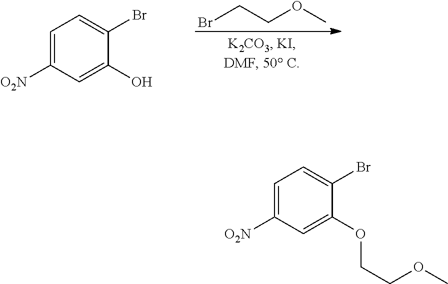

A mixture of 2-bromo-5-nitro-phenol (1.0 g, 5 mmol, 1.0 eq.), 1-bromo-2-methoxy-ethane (956 mg, 7 mmol, 1.5 eq.), KI (152 mg, 917 umol, 0.2 eq.) and Cs₂CO₃ (2.2 g, 7 mmol, 1.5 eq.) in DMF (20 mL) was stirred at 25° C. for 12 hrs under N₂ atmosphere. The reaction mixture was quenched by H₂O (20 mL), filtered, and concentrated to yield 1-bromo-2-(2-methoxyethoxy)-4-nitrobenzene (1.1 g, crude) as a white solid.

### Step 2: Synthesis of 4-bromo-3-(2-methoxyethoxy)aniline

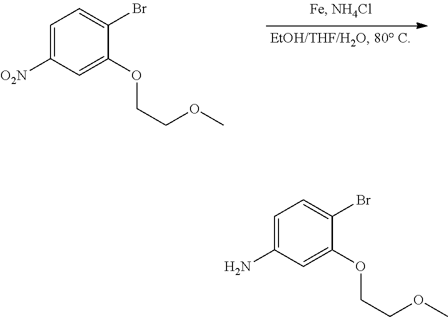

A mixture of 1-bromo-2-(2-methoxyethoxy)-4-nitrobenzene (1.1 g, 4 mmol, 1.0 eq.), Fe (1.1 g, 20 mmol, 5.0 eq.) and NH₄Cl (639 mg, 12 mmol, 3.0 eq.) in EtOH (18 mL)/THF (18 mL)/H₂O (6 mL) was stirred at 80° C. for 12 hrs under N₂ atmosphere, filtered, concentrated, quenched by H₂O (100 mL) and extracted with EtOAc (150 mL×3). The combined organic layers were dried over Na₂SO₄, filtered, concentrated and purified by column chromatography (SiO₂, Petroleum ether:EtOAc=1:0 to 0:1) to yield 4-bromo-3-(2-methoxyethoxy)aniline (810 mg, 3 mmol, 82.6% yield) as a white solid.

ESI [M+H]=246.1/248.1

### Step 3: Synthesis of isopropyl (4-bromo-3-(2-methoxyethoxy)phenyl)carbamate

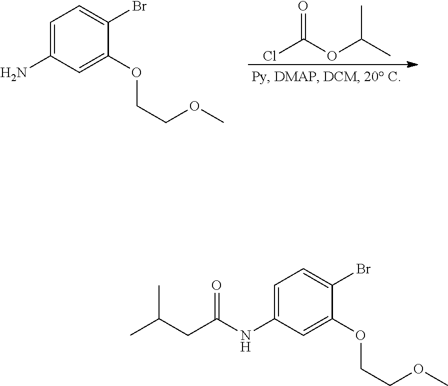

To a solution of isopropyl chloroformate (373 mg, 3 mmol, 1.5 eq.) in DCM (20 mL) was added Py (482 mg, 6 mmol, 3.0 eq.), 4-bromo-3-(2-methoxyethoxy)aniline (500 mg, 2 mmol, 1.0 eq.) and DMAP (25 mg, 203 umol, 0.1 eq.). The mixture was stirred at 20° C. for 0.2 hr, quenched by 2N HCl (20 mL) and extracted with DCM (60 mL*3). The combined organic layers were dried over Na₂SO₄, filtered, concentrated and purified by column chromatography (SiO₂, Petroleum ether:EtOAc=10:0 to 0:1) to yield isopropyl (4-bromo-3-(2-methoxyethoxy)phenyl)carbamate (550 mg, 2 mmol, 81.5% yield) as a white solid. ESI [M+H]=332.1/334.1

### Step 4: Synthesis of isopropyl N-[3-(2-methoxyethoxy)-4-(4,4,5,5-tetramethyl-1,3,2-dioxaborolan-2-yl)phenyl]carbamate

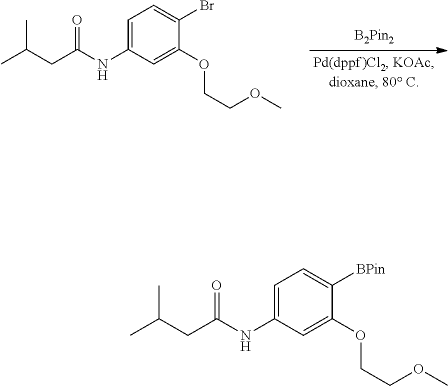

A mixture of 4,4,5,5-tetramethyl-2-(4,4,5,5-tetramethyl-1,3,2-dioxaborolan-2-yl)-1,3,2-dioxaborolane (1.0 g, 4 mmol, 3.0 eq.), isopropyl (4-bromo-3-(2-methoxyethoxy)phenyl)carbamate (450 mg, 1 mmol, 1.0 eq.), KOAc (399 mg, 4 mmol, 3.0 eq.) and Pd(dppf)Cl₂ (99 mg, 135 umol, 0.1 eq.) in dioxane (20 mL) stirred at 80° C. for 12 hrs under N₂ atmosphere, concentrated and purified by column chromatography (SiO₂, Petroleum ether:EtOAc=30:1 to 5:1) and prep-TLC (SiO₂, Petroleum ether:EtOAc=5:1) to yield isopropyl N-[3-(2-methoxyethoxy)-4-(4,4,5,5-tetramethyl-1,3,2-dioxaborolan-2-yl)phenyl]carbamate (300 mg, 791 umol, 58.4% yield) as a white solid.

ESI [M+H]=380.2

### Step 5: Synthesis of isopropyl ((trans)-4-(5-(4-((isopropoxycarbonyl)amino)-2-(2-methoxyethoxy)phenyl)thiazol-2-yl)cyclohexyl)carbamate (13)

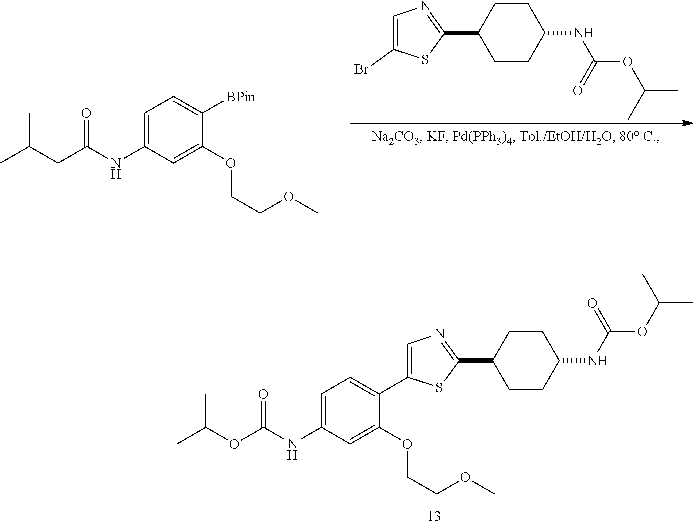

A mixture of isopropyl trans-N-[4-(5-bromothiazol-2-yl)cyclohexyl]carbamate (101 mg, 290 umol, 1.1 eq isopropyl N-[3-(2-methoxyethoxy)-4-(4,4,5,5-tetramethyl-1,3,2-dioxaborolan-2-yl)phenyl]carbamate (100 mg, 264 umol, 1.0 eq.), Na₂CO₃ (56 mg, 527 umol, 2.0 eq.), KF (23 mg, 396 umol, 1.5 eq.) and Pd(PPh₃)₄ (30 mg, 26 umol, 0.1 eq.) in Tol. (3 mL)/EtOH (3 mL)/H₂O (1 mL) was stirred at 80° C. for 4 hrs under N₂ atmosphere. The reaction mixture was diluted with H₂O (15 mL) and extracted with EtOAc (30 mL×3). The combined organic layers were dried over Na₂SO₄, filtered, concentrated and purified by prep-HPLC (column: Nano-Micro UniSil 5-100 C18 ULTRA 100×250 mm 5 um; mobile phase: [water(0.1% TFA)-ACN]; B %: 45%-65%, 10 mins) to yield isopropyl ((trans)-4-(5-(4-((isopropoxycarbonyl)amino)-2-(2-methoxyethoxy)phenyl)thiazol-2-yl)cyclohexyl)carbamate (18 mg, 34 umol, 12.9% yield, 100.0% purity) as pale yellow solid. ¹H NMR (400 MHz, methanol-d4) δ=8.13 (s, 1H), 7.57 (br d, J=8.44 Hz, 1H), 7.39 (br s, 1H), 7.05 (br d, J=8.31 Hz, 1H), 4.93-5.15 (m, 2H), 4.20-4.33 (m, 2H), 3.87 (br s, 2H), 3.42-3.56 (m, 4H), 3.04 (br s, 1H), 2.23 (br d, J=11.74 Hz, 2H), 2.10 (br d, J=10.51 Hz, 2H), 1.71 (br d, J=12.59 Hz, 2H), 1.39-1.50 (m, 2H), 1.33 (br d, J=5.87 Hz, 6H), 1.21-1.29 (m, 6H). ESI [M+H]=520.2

### Example 14: Preparation of isopropyl ((trans)-4-(5-(4-((isopropoxy carbonyl)amino)-2-methoxyphenyl)thiazol-2-yl)cyclohexyl)carbamate

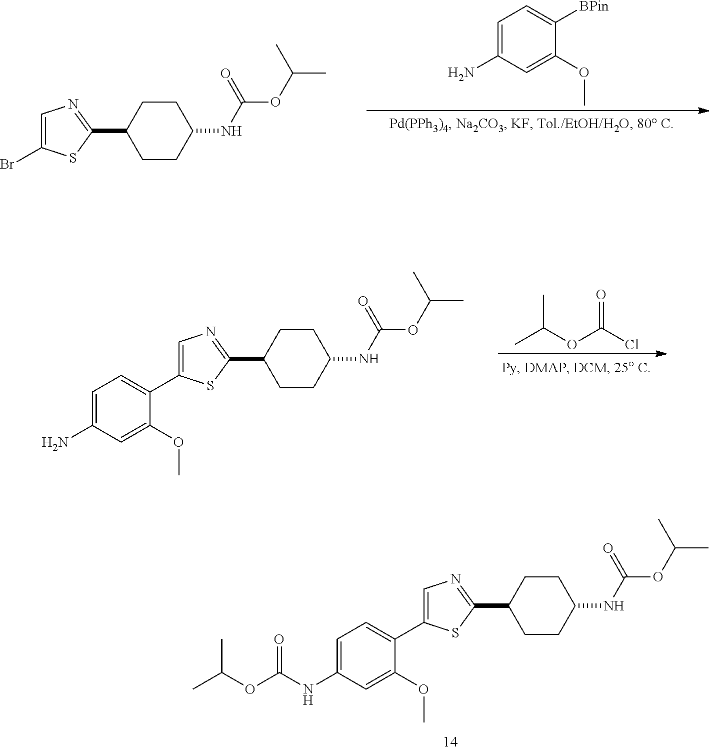

### Step 1: Synthesis of isopropyl ((trans)-4-(5-(4-amino-2-methoxyphenyl)thiazol-2-yl)cyclohexyl)carbamate

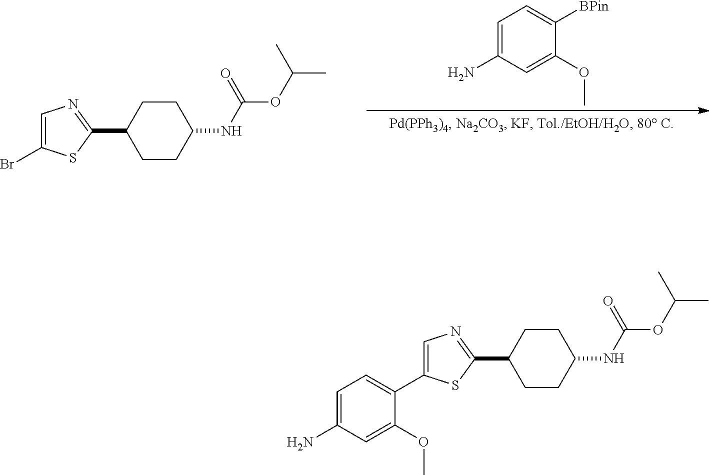

A mixture of 3-methoxy-4-(4,4,5,5-tetramethyl-1,3,2-dioxaborolan-2-yl)aniline (50 mg, 201 umol, 1.2 eq.), isopropyl trans-N-[4-(5-bromothiazol-2-yl)cyclohexyl]carbamate (58 mg, 167 umol, 1.0 eq.), KF (15 mg, 251 umol, 1.5 eq.), Pd(PPh₃)₄ (19 mg, 17 umol, 0.1 eq.) and Na₂CO₃ (53 mg, 502 umol, 3.0 eq.) in Tol. (1 mL)/EtOH (1 mL)/H₂O (0.3 mL) was stirred at 80° C. for 0.5 hr under N₂ atmosphere. The mixture was quenched by H₂O (5 mL), concentrated and extracted with EtOAc (10 mL×3). The combined organic layers were dried over Na₂SO₄, filtered, concentrated and purified by prep-TLC (Petroleum ether:EtOAc=0:1) to yield isopropyl trans-N-[4-[5-(4-amino-2-methoxy-phenyl)thiazol-2-yl]cyclohexyl]carbamate (65 mg, crude) as a yellow solid. ESI [M+H]=390.3

### Step 2: Synthesis of isopropyl ((trans)-4-(5-(4-((isopropoxy carbonyl)amino)-2-methoxyphenyl)thiazol-2-yl)cyclohexyl)carbamate (14)

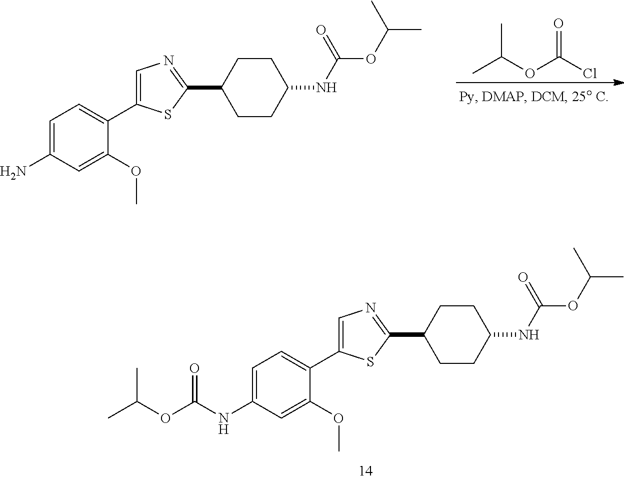

To a solution of isopropyl trans-N-[4-[5-(4-amino-2-methoxy-phenyl)thiazol-2-yl]cyclohexyl]carbamate (65 mg, 167 umol, 1.0 eq.) in DCM (3 mL) was added pyridine (40 mg, 502 mol, 3.0 eq.), DMAP (2 mg, 17 umol, 0.1 eq.) and isopropyl chloroformate (61 mg, 502 umol, 3.0 eq.). The mixture was stirred at 25° C. for 1 hr, quenched by 1N HCl (10 mL) and extracted with DCM (30 mL×3). The combined organic layers were washed with sat. aq. NaHCO₃ (5 mL×3), dried over Na₂SO₄, filtered, concentrated and purified by prep-HPLC (column: Welch Ultimate AQ-C18 150×30 mm×5 um; mobile phase: [water(TFA)-ACN]; B %: 45%-75%, 12 mins) to yield isopropyl ((trans)-4-(5-(4-amino-2-methoxyphenyl)thiazol-2-yl)cyclohexyl) carbamate (14) (52 mg, 110 umol, 65.6% yield, 100.0% purity) as a white solid.

¹H NMR (400 MHz, methanol-d4) δ=8.09 (s, 1H), 7.59 (d, J=8.4 Hz, 1H), 7.42 (s, 1H), 7.06 (dd, J=1.7, 8.4 Hz, 1H), 5.06-4.95 (m, 1H), 4.88-4.79 (m, 1H), 3.97 (s, 3H), 3.48 (ddd, J=4.0, 7.7, 11.6 Hz, 1H), 3.12-2.93 (m, 1H), 2.22 (br d, J=12.8 Hz, 2H), 2.10 (br d, J=10.5 Hz, 2H), 1.79-1.65 (m, 2H), 1.50-1.37 (m, 2H), 1.33 (d, J=6.2 Hz, 6H), 1.25 (br d, J=6.1 Hz, 6H). ESI [M+H]=476.2

### Example 15: Preparation of isopropyl ((trans)-4-(5-(2-(2-hydroxyacetamido)-4-((isopropoxycarbonyl)amino)phenyl)thiazol-2-yl)cyclohexyl)carbamate

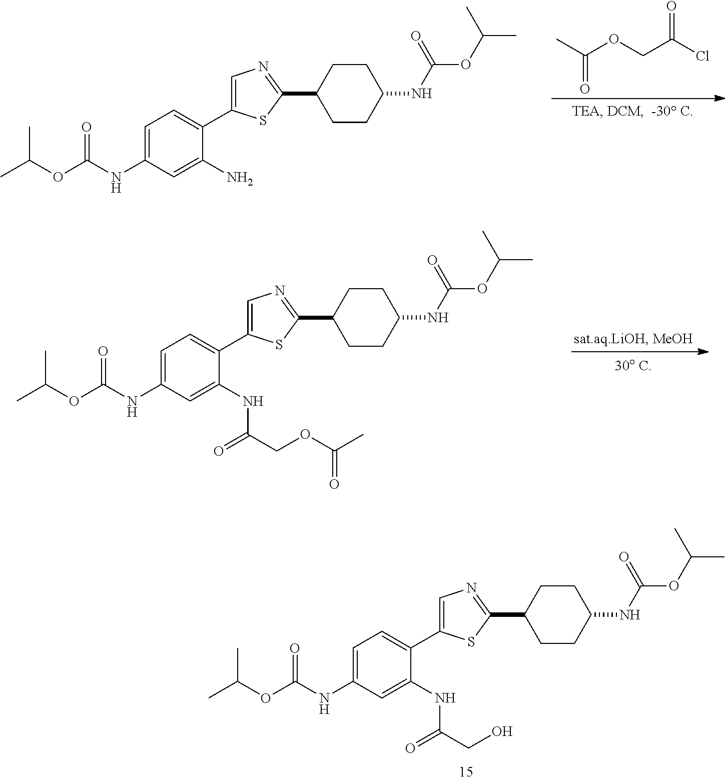

### Step 1: Synthesis of trans-[2-[5-(isopropoxycarbonylamino)-2-[2-[4-(isopropoxy carbonylamino)cyclohexyl]thiazol-5-yl]anilino]-2-oxo-ethyl]acetate

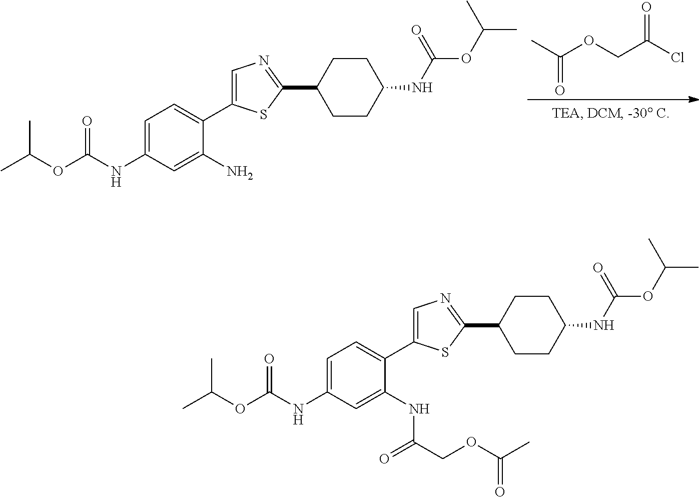

To a solution of isopropyl trans-N-[3-amino-4-[2-[4-(isopropoxycarbonyl amino)cyclohexyl]thiazol-5-yl]phenyl]carbamate (30 mg, 65 umol, 1.0 eq.) in DCM (2 mL) was added TEA (19 mg, 195 umol, 3.0 eq.) and (2-chloro-2-oxo-ethyl) acetate (13 mg, 97 umol, 1.5 eq.). The mixture was stirred at −30° C. for 0.5 hr, quenched by 1N HCl (10 mL) and extracted with DCM (30 mL×2). The combined organic layers were washed with sat. aq. NaHCO₃ (10 mL), dried over Na₂SO₄, filtered and concentrated to yield trans-[2-[5-(isopropoxycarbonylamino)-2-[2-[4-(isopropoxycarbonylamino)cyclohexyl]thiazol-5-yl]anilino]-2-oxo-ethyl]acetate (18 mg, crude) as a white solid.

ESI [M+H]=561.3

### Step 2: Synthesis of isopropyl ((trans)-4-(5-(2-(2-hydroxyacetamido)-4-((iso propoxycarbonyl)amino)phenyl)thiazol-2-yl)cyclohexyl)carbamate (15)

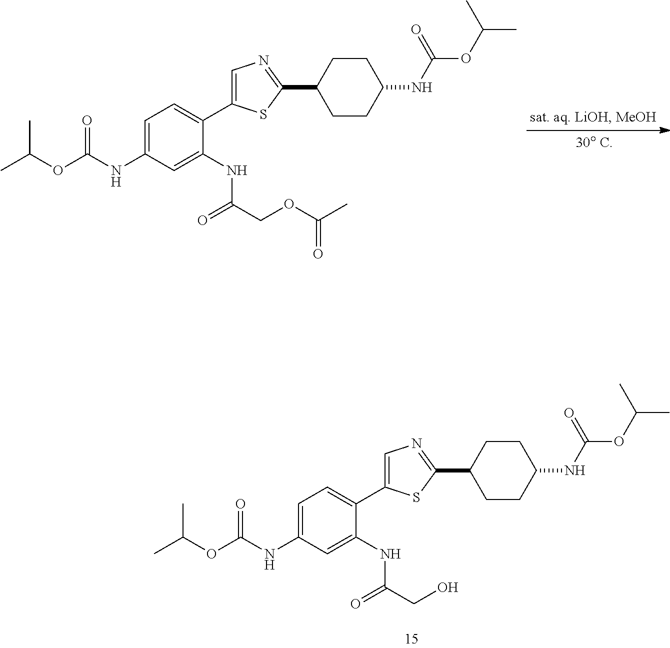

To a solution of trans-[2-[5-(isopropoxycarbonylamino)-2-[2-[4-(isopropoxy carbonylamino)cyclohexyl]thiazol-5-yl]anilino]-2-oxo-ethyl]acetate (15 mg, 26 umol, 1.0 eq.) in MeOH (2 mL) was added sat. aq. LiOH (2 mL). The mixture was stirred at 30° C. for 3 hrs, concentrated and extracted with EtOAc (10 mL×3). The combined organic layers were dried over Na₂SO₄, filtered, concentrated and purified by prep-HPLC (column: Nano-Micro UniSil 5-100 C18 ULTRA 100×250 mm Sum; mobile phase: [water(TFA)-ACN]; B %: 40%-52%, 10 mins) to yield isopropyl ((trans)-4-(5-(2-(2-hydroxyacetamido)-4-((isopropoxycarbonyl)amino)phenyl)thiazol-2-yl)cyclohexyl)carbamate (15) (3 mg, 7 umol, 26.4% yield, 99.0% purity) as a white solid. ¹H NMR (400 MHz, methanol-d4) δ ppm 8.03 (s, 1H), 7.75 (s, 1H), 7.44 (s, 2H), 4.98 (dt, J=12.35, 6.30 Hz, 1H), 4.80-4.85 (m, 1H), 4.10 (s, 2H), 3.44-3.51 (m, 1H), 2.97-3.07 (m, 1H), 2.19-2.27 (m, 2H), 2.05-2.12 (m, 2H), 1.65-1.78 (m, 2H), 1.37-1.48 (m, 2H), 1.32 (d, J=6.24 Hz, 6H), 1.24 (br d, J=6.11 Hz, 6H). ESI [M+H]=519.2

### Example 16: Preparation of isopropyl ((trans)-4-(5-(2-(3-(2-hydroxyethyl)ureido)-4-((isopropoxycarbonyl)amino)phenyl)thiazol-2-yl)cyclohexyl)carbamate

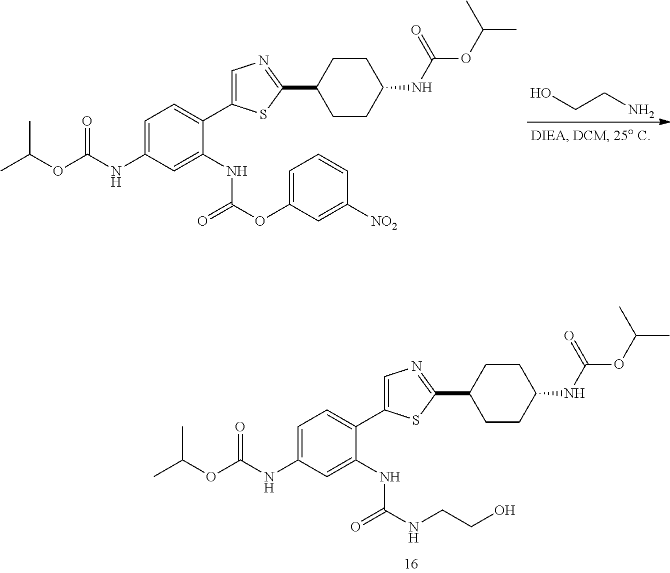

Following the same protocol and under the same conditions as for example 8, step 2, using 2-hydroxyethylamine, isopropyl ((trans)-4-(5-(2-(3-(2-hydroxyethyl)ureido)-4-((isopropoxycarbonyl)amino)phenyl)thiazol-2-yl)cyclohexyl)carbamate (16) was obtained (39 mg, 71 umol, 25.5% yield) as a white solid. ¹H NMR (400 MHz, methanol-d4) δ=7.75 (s, 1H), 7.70 (d, J=1.96 Hz, 1H), 7.32-7.45 (m, 2H), 4.94-5.01 (m, 1H), 4.80-4.87 (m, 1H), 3.59 (t, J=5.62 Hz, 2H), 3.42-3.51 (m, 1H), 3.29 (t, J=5.69 Hz, 2H), 2.95-3.05 (m, 1H), 2.19-2.26 (m, 2H), 2.04-2.12 (m, 2H), 1.71 (qd, J=12.88, 2.57 Hz, 2H), 1.42 (qd, J=12.53, 2.87 Hz, 2H), 1.32 (d, J=6.24 Hz, 6H), 1.24 (br d, J=6.11 Hz, 6H). ESI [M+H]=548.3

### Example 17: Preparation of isopropyl ((trans)-4-(5-(4-((isopropoxycarbonyl)amino)-2-(piperazin-1-yl)phenyl)thiazol-2-yl)cyclohexyl)carbamate

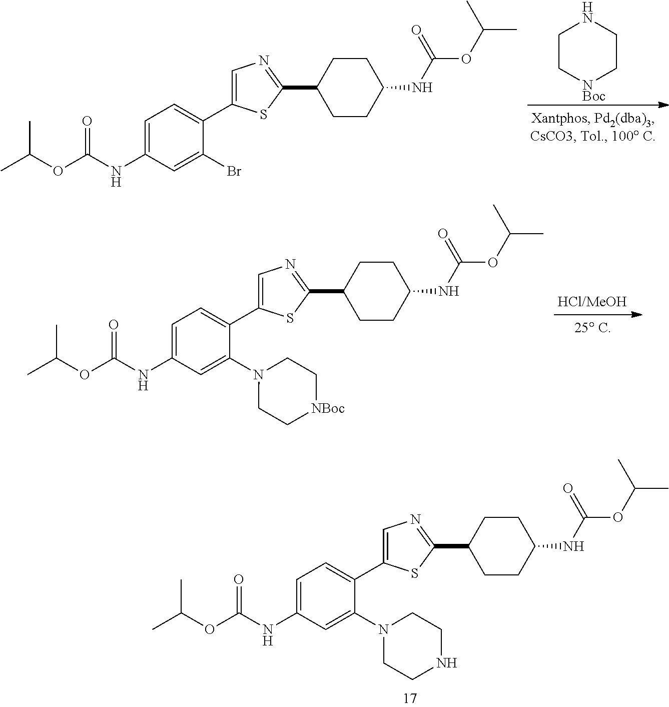

### Step 1: Synthesis of tert-butyl 4-(5-((isopropoxycarbonyl)amino)-2-(2-((trans)-4-((isopropoxycarbonyl)amino)cyclohexyl)thiazol-5-yl)phenyl)piperazine-1-carboxylate

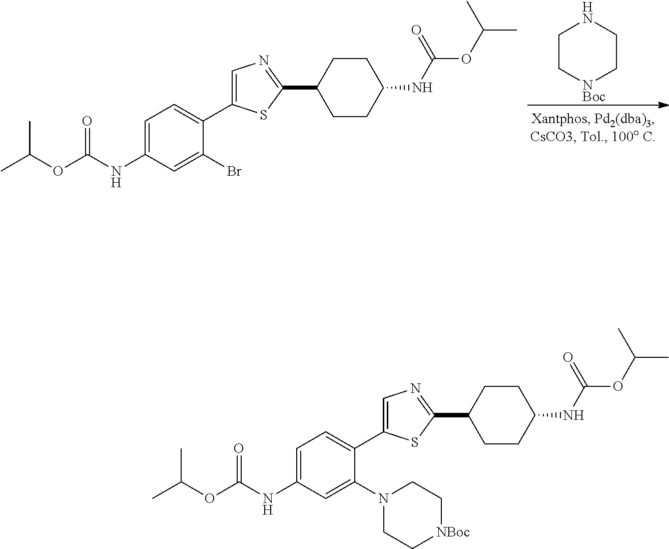

Following the same protocol and under the same conditions as for example 1, step 7, using tert-butyl piperazine-1-carboxylate, tert-butyl 4-(5-((isopropoxy carbonyl)amino)-2-(2-((trans)-4-((isopropoxycarbonyl)amino)cyclohexyl)thiazol-5-yl)phenyl)piperazine-1-carboxylate was obtained (50 mg, 79 umol, 83.3% yield) as a yellow oil. ESI [M+H]=630.4

### Step 2: Synthesis of isopropyl ((trans)-4-(5-(4-((isopropoxycarbonyl)amino)-2-(piperazin-1-yl)phenyl)thiazol-2-yl)cyclohexyl)carbamate (17)

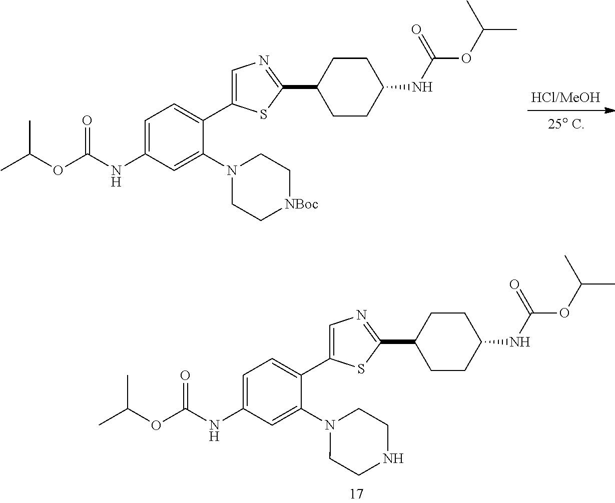

A mixture of tert-butyl 4-(5-((isopropoxy carbonyl)amino)-2-(2-((trans)-4-((isopropoxycarbonyl)amino)cyclohexyl)thiazol-5-yl)phenyl)piperazine-1-carboxylate (50 mg, 79 umol, 1.0 eq.) in HCl/MeOH (4 M, 3 mL) was stirred at 25° C. The mixture was concentrated and purified by prep-HPLC (column: Nano-Micro UniSil 5-100 C18 ULTRA 100×250 mm Sum; mobile phase: [water(0.1% TFA)-ACN]; B %: 25%-45%, 10 mins) to yield isopropyl ((trans)-4-(5-(4-((isopropoxycarbonyl)amino)-2-(piperazin-1-yl)phenyl)thiazol-2-yl)cyclohexyl)carbamate (17) (4 mg, 6 umol, 7.7% yield, 97.7% purity, TFA) as a pale yellow solid. ¹H NMR (400 MHz, methanol-d4) δ=7.95 (s, 1H), 7.64 (s, 1H), 7.52 (d, J=8.77 Hz, 1H), 7.15 (dd, J=1.75, 8.33 Hz, 1H), 4.96 (quin, J=6.25 Hz, 1H), 4.77-4.87 (m, 1H), 3.43-3.53 (m, 1H), 3.35-3.42 (m, 4H), 3.09-3.17 (m, 4H), 3.01 (br t, J=11.84 Hz, 1H), 2.21 (br d, J=13.15 Hz, 2H), 2.07 (br d, J=9.65 Hz, 2H), 1.63-1.78 (m, 2H), 1.35-1.49 (m, 2H), 1.31 (d, J=6.14 Hz, 6H), 1.22 (d, J=6.58 Hz, 6H). ESI [M+H]=530.3

### Example 18: Preparation of isopropyl ((trans)-4-(5-(4-((isopropoxycarbonyl)amino)-2-((N-methylmethylsulfonamido)methyl)phenyl)thiazol-2-yl)cyclohexyl) carbamate

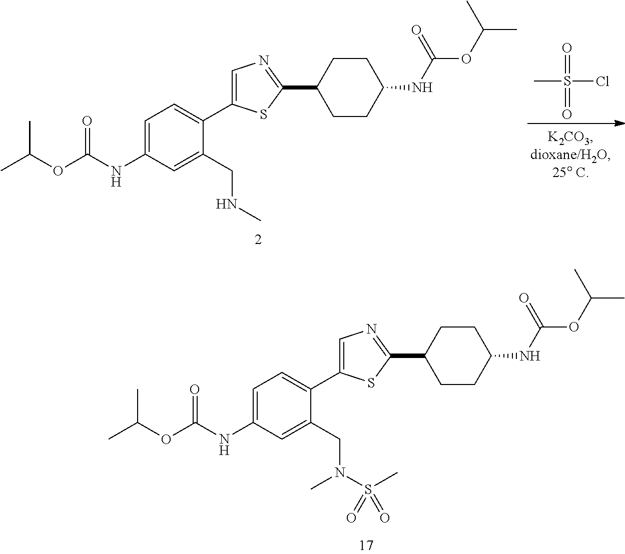

To a mixture of isopropyl ((trans)-4-(5-(4-((isopropoxycarbonyl)amino)-2-((methyl-amino)methyl)phenyl)thiazol-2-yl)cyclohexyl)carbamate (2) (6 mg, 9 umol, 1.0 eq., TFA) and K₂CO₃ (1 mg, 9 umol, 1.0 eq.) in dioxane (2 mL)/H₂O (0.5 mL) was added methanesulfonyl chloride (2 mg, 14 umol, 1.5 eq.). The mixture was stirred at 25° C. for 0.5 hr under N₂ atmosphere, quenched by H₂O (10 mL), extracted with EtOAc (20 mL×3). The combined organic layers were dried over Na₂SO₄, filtered, concentrated and purified by prep-HPLC (column: Nano-Micro UniSil 5-100 C18 ULTRA 100×250 mm Sum; mobile phase: [water(0.1% TFA)-ACN]; B %: 47%-63%, 10 mins) to yield isopropyl ((trans)-4-(5-(4-((isopropoxycarbonyl)amino)-2-((N-methylmethyl-sulfonamido)methyl)phenyl)thiazol-2-yl)cyclohexyl) carbamate (4 mg, 6 umol, 67.7% yield, 99.0% purity) as a pale yellow solid. ¹HNMR (methanol-d4, 400 MHz) δ=7.68 (d, J=2.1 Hz, 1H), 7.62 (d, J=5.0 Hz, 1H), 7.53 (br d, J=7.2 Hz, 1H), 7.33 (d, J=8.4 Hz, 1H), 4.9-5.0 (m, 1H), 4.8-4.8 (m, 1H), 4.37 (s, 2H), 3.4-3.5 (m, 1H), 3.0-3.1 (m, 1H), 2.89 (s, 3H), 2.69 (s, 3H), 2.24 (br d, J=12.6 Hz, 2H), 2.09 (br d, J=10.6 Hz, 2H), 1.6-1.8 (m, 2H), 1.4-1.5 (m, 2H), 1.33 (d, J=6.2 Hz, 6H), 1.24 (br d, J=6.1 Hz, 6H). ESI [M+H]=567.2

### Example 19: Preparation of isopropyl ((trans)-4-(5-(2-amino-4-(3-benzylureido)pheny l)thiazol-2-yl)cyclohexyl)carbamate

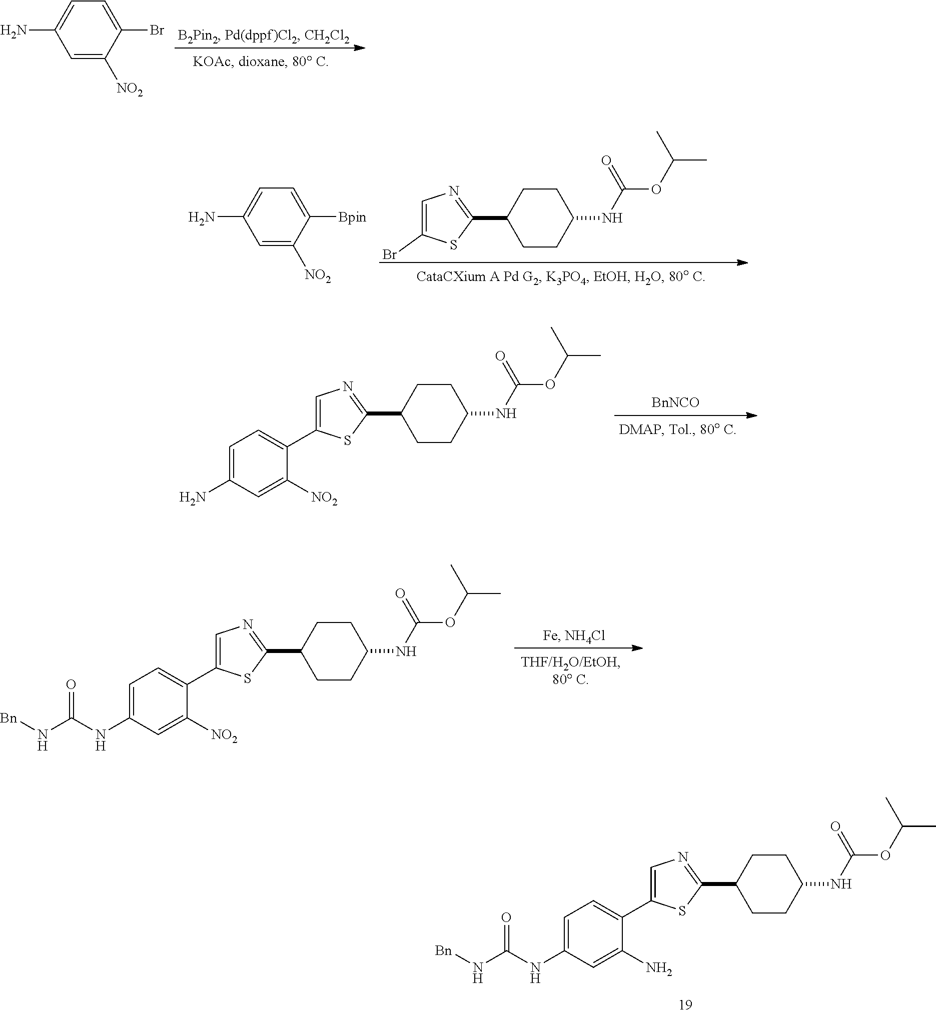

### Step 1: Synthesis of 3-nitro-4-(4,4,5,5-tetramethyl-1,3,2-dioxaborolan-2-yl)aniline

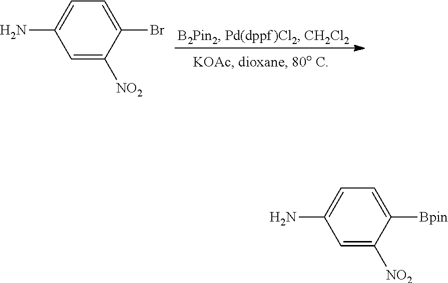

A mixture of 4-bromo-3-nitro-aniline (780 mg, 3 mmol, 1.0 eq.), 4,4,5,5-tetramethyl-2-(4,4,5,5-tetramethyl-1,3,2-dioxaborolan-2-yl)-1,3,2-dioxaborolane (456 mg, 1 mmol, 0.5 eq.), KOAc (1.0 g, 10 mmol, 3.0 eq.) and Pd(dppf)Cl₂·CH₂Cl₂ (88 mg, 107 umol, 0.03 eq.) in dioxane (15 mL) was stirred at 80° C. for 12 hrs under N₂ atmosphere, concentrated and purified by column chromatography (SiO₂, Petroleum ether:EtOAc=50:1 to 2:1) to yield 3-nitro-4-(4,4,5,5-tetramethyl-1,3,2-dioxaborolan-2-yl)aniline (1.0 g, crude) as a yellow oil. ESI [M+H]=265.1

### Step 2: Synthesis of isopropyl trans-N-[4-[5-(4-amino-2-nitro-phenyl)thiazol-2-yl]cyclohexyl]carbamate

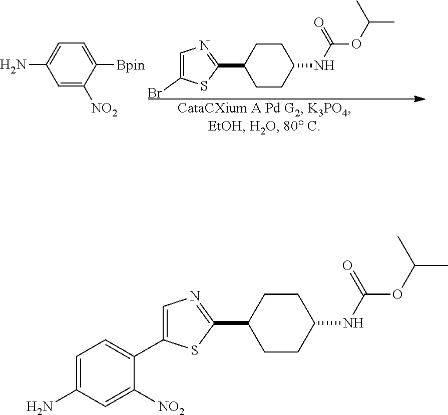

A mixture of 3-nitro-4-(4,4,5,5-tetramethyl-1,3,2-dioxaborolan-2-yl)aniline 1.0 g, 3 mmol, 1.2 eq.), isopropyl trans-N-[4-(5-bromothiazol-2-yl)cyclohexyl]carbamate (1.1 g, 3 mmol, 1.0 eq.), aq. K₃PO₄ (0.3 M, 31.56 mL, 3.0 eq.) and CXium A Pd G2 (211 mg, 315 umol, 0.1 eq.) in EtOH (20 mL) was stirred at 80° C. for 12 hrs under N₂ atmosphere, quenched by H₂O (20 mL) and concentrated to remove EtOH. The residue was extracted with EtOAc (20 mL×3), dried over Na₂SO₄, filtered, concentrated and purified by column chromatography (SiO₂, Petroleum ether:EtOAc=100:1 to 3:1) to yield isopropyl trans-N-[4-[5-(4-amino-2-nitro-phenyl)thiazol-2-yl]cyclohexyl]carbamate (400 mg, 988 umol, 31.3% yield) as a yellow solid. ¹H NMR (400 MHz, methanol-d4) δ=7.44 (s, 1H), 7.21 (d, J=8.4 Hz, 1H), 7.08 (d, J=2.4 Hz, 1H), 6.91-6.85 (m, 1H), 4.81-4.76 (m, 1H), 3.50-3.38 (m, 1H), 3.01-2.88 (m, 1H), 2.19 (br d, J=12.6 Hz, 2H), 2.11-1.99 (m, 2H), 1.66 (dq, J=3.0, 12.8 Hz, 2H), 1.48-1.33 (m, 2H), 1.27-1.17 (m, 6H). ESI [M+H]=405.1

### Step 3: Synthesis of isopropyl trans-N-[4-[5-[4-(benzylcarbamoylamino)-2-nitro-phenyl]thiazol-2-yl]cyclohexyl]carbamate

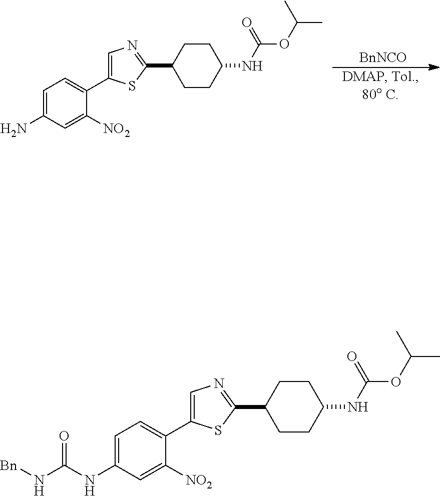

To a solution of isopropyl trans-N-[4-[5-(4-amino-2-nitro-phenyl)thiazol-2-yl]cyclohexyl]carbamate (370 mg, 914 umol, 1.0 eq.) in Tol. (5 mL) was added DMAP (11 mg, 91 umol, 0.1 eq.) and BnNCO (182 mg, 1 mmol, 1.5 eq.). The mixture was stirred at 80° C. for 1 hr, quenched by sat. aq. Na₂CO₃ (10 mL) at 0° C., and extracted with EtOAc (100 mL×3). The organic layers were dried over Na₂SO₄, filtered, concentrated and triturated with Petroleum ether:EtOAc=1:1 (15 mL) to yield isopropyl trans-N-[4-[5-[4-(benzylcarbamoylamino)-2-nitro-phenyl]thiazol-2-yl]cyclohexyl]carbamate (300 mg, 558 umol, 61.0% yield) as a yellow solid. ¹H NMR (400 MHz, DMSO-d6) δ=9.25 (s, 1H), 8.21 (s, 1H), 7.65-7.59 (m, 1H), 7.52 (br d, J=8.4 Hz, 1H), 7.37-7.29 (m, 5H), 7.06-6.86 (m, 2H), 4.82-4.69 (m, 1H), 4.33 (br d, J=5.6 Hz, 2H), 4.24 (br d, J=5.9 Hz, 1H), 3.00-2.91 (m, 1H), 2.12 (br d, J=11.7 Hz, 2H), 1.91 (br d, J=9.7 Hz, 2H), 1.63-1.49 (m, 2H), 1.44-1.27 (m, 2H), 1.17 (br d, J=6.1 Hz, 6H). ESI [M+H]=538.3

### Step 4: Synthesis of isopropyl ((trans)-4-(5-(2-amino-4-(3-benzylureido)phenyl)thiazol-2-yl)cyclohexyl)carbamate (19)

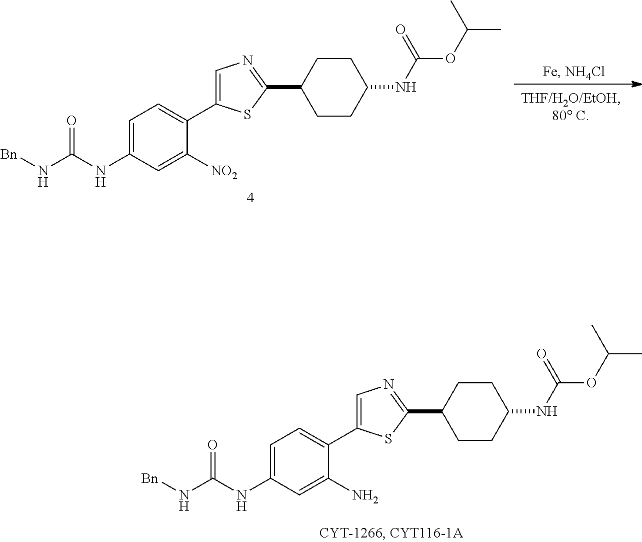

To a solution of isopropyl trans-N-[4-[5-[4-(benzylcarbamoylamino)-2-nitro-phenyl]thiazol-2-yl]cyclohexyl]carbamate (290 mg, 539 umol, 1.0 eq.) in EtOH (2 mL)/THF (2 mL)/H₂O (0.6 mL) was added Fe (150 mg, 2 mmol, 5.0 eq.) and NH₄Cl (57 mg, 1 mmol, 2.0 eq.). The mixture was stirred at 80° C. for 3 hrs and concentrated. The residue was diluted with H₂O (15 mL) and extracted with EtOAc (150 mL×3). The organic layers were dried over Na₂SO₄, filtered, concentrated and purified by prep-HPLC (column: Phenomenex Luna C18 200×40 mm×10 um; mobile phase: [water(TFA)-ACN]; B %: 30%-50%, 10 mins) to yield isopropyl ((trans)-4-(5-(2-amino-4-(3-benzylureido)phenyl)thiazol-2-yl)cyclohexyl)carbamate (19) (150 mg, 296 umol, 55.0% yield, 100.0% purity) as a pale yellow solid. ¹H NMR (400 MHz, methanol-d4) δ=7.75-7.67 (m, 1H), 7.42 (br d, J=1.8 Hz, 1H), 7.35 (d, J=4.4 Hz, 4H), 7.30-7.26 (m, 1H), 7.26-7.22 (m, 1H), 6.94 (dd, J=2.1, 8.4 Hz, 1H), 5.02 (br d, J=3.1 Hz, 1H), 4.42 (s, 2H), 3.55-3.41 (m, 1H), 3.04 (tt, J=3.5, 12.0 Hz, 1H), 2.32-2.19 (m, 2H), 2.14-2.02 (m, 2H), 1.72 (dq, J=3.0, 12.9 Hz, 2H), 1.51-1.36 (m, 2H), 1.25 (br d, J=6.2 Hz, 6H). ESI [M+H]=508.2

### Example 20: Preparation of isopropyl ((trans)-4-(5-(4-(3-benzylureido)-2-hydroxyphenyl)thiazol-2-yl)cyclohexyl)carbamate

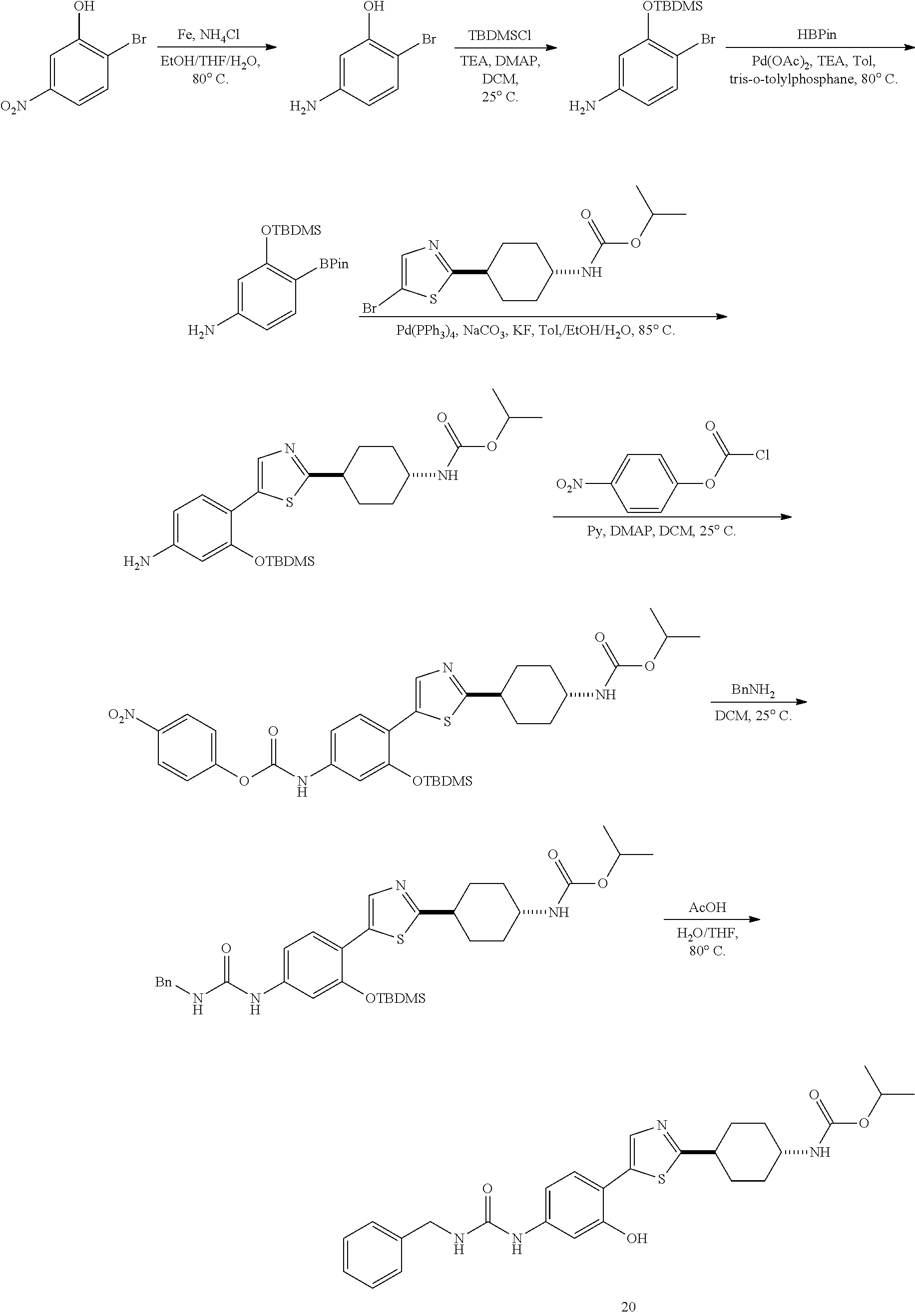

### Step 1: Synthesis of 5-amino-2-bromophenol

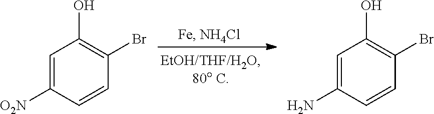 [SMILES: O=[N+]([O-])c1ccc(Br)c(O)c1 ; Nc1ccc(Br)c(O)c1]

To a solution of 2-bromo-5-nitrophenol (0.4 g, 2 mmol, 1.0 eq.) in THF (3 mL)/EtOH (3 mL)/H₂O (1 mL) was added NH₄Cl (258 mg, 5 mmol, 3.0 eq.) and Fe (269 mg, 5 mmol, 3.0 eq.). The mixture was stirred at 80° C. for 1 hr, filtered and concentrated under reduced pressure to give a residue. The reaction mixture was diluted with H₂O (50 mL) and extracted with EtOAc (50 mL×3). The combined organic layers were washed with brine (20 mL), dried over Na₂SO₄, filtered, and concentrated under reduced pressure to yield 5-amino-2-bromophenol (0.4 g, crude) as a gray solid. ESI [M+H]=188.1/190.1

### Step 2: Synthesis of 4-bromo-3-[tert-butyl(dimethyl)silyl]oxy-aniline

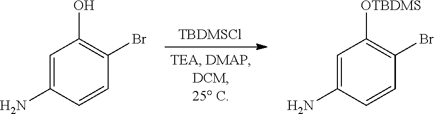

To a solution of 5-amino-2-bromophenol (0.4 g, 2 mmol, 1.0 eq.) in DCM (5 mL) was added TEA (188 mg, 2 mmol, 1.0 eq.), DMAP (23 mg, 186 umol, 0.1 eq.) and TBDMSCl (561 mg, 4 mmol, 2.0 eq.). The mixture was stirred at 25° C. for 1 hr, quenched by H₂O (50 mL) and extracted with DCM (50 mL×3). The combined organic layers were washed with sat. aq. NaCl (100 mL), dried over Na₂SO₄, filtered, concentrated under reduced pressure and purified by prep-TLC (SiO₂, Petroleum ether:EtOAc=5:1) to yield 4-bromo-3-[tert-butyl(dimethyl)silyl]oxy-aniline (0.4 g, crude) as a yellow oil. ESI [M+H]=302.1/304.1

### Step 3: Synthesis of 3-[tert-butyl(dimethyl)silyl]oxy-4-(4,4,5,5-tetramethyl-1,3,2-dioxaborolan-2-yl)aniline

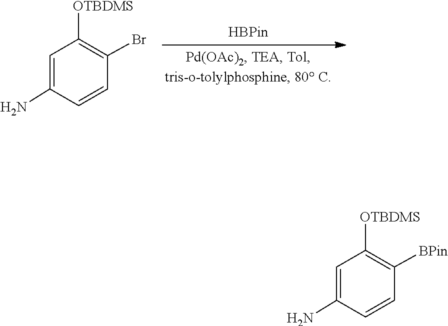

A mixture of 4-bromo-3-[tert-butyl(dimethyl)silyl]oxy-aniline (0.2 g, 662 umol, 1.0 eq.), 4,4,5,5-tetramethyl-1,3,2-dioxaborolane (339 mg, 3 mmol, 4.0 eq.), Pd(OAc)₂ (15 mg, 66 umol, 0.1 eq.), tris-o-tolylphosphine (20 mg, 66 umol, 0.1 eq.) and TEA (167 mg, 2 mmol, 2.5 eq.) in Tol. (2 mL) was stirred at 80° C. for 12 hrs under N₂ atmosphere and concentrated under reduced pressure to give a residue. The reaction mixture was diluted with H₂O (100 mL) and extracted with EtOAc (100 mL×3). The combined organic layers were washed with brine (100 mL), dried over Na₂SO₄, filtered, concentrated under reduced pressure and purified by column chromatography (SiO₂, Petroleum ether:EtOAc=20:1 to 10:1) to yield 3-[tert-butyl(dimethyl)silyl]oxy-4-(4,4,5,5-tetramethyl-1,3,2-dioxaborolan-2-yl)aniline (0.5 g, crude) as a yellow oil. [0551]¹H NMR (400 MHz, CDCl₃) δ=7.28 (d, J=7.9 Hz, 1H), 6.10-6.02 (m, 1H), 5.87 (d, J=2.1 Hz, 1H), 1.08 (s, 12H), 0.82-0.79 (m, 9H), 0.01-0.01 (m, 6H). ESI [M+H]=350.2

### Step 4: Synthesis of isopropyl trans-N-[4-[5-(4-amino-2-hydroxy-phenyl)thiazol-2-yl]cyclohexyl]carbamate

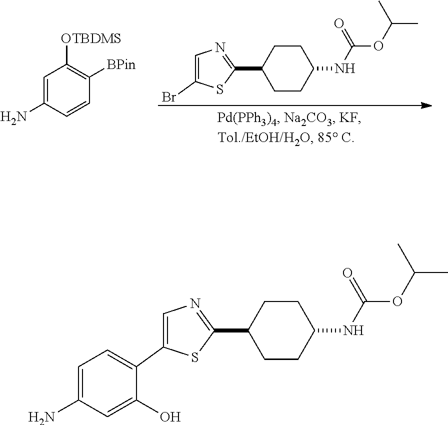

A mixture of 3-[tert-butyl(dimethyl)silyl]oxy-4-(4,4,5,5-tetramethyl-1,3,2-dioxaborolan-2-yl)aniline (332 mg, 950 umol, 1.1 eq.), isopropyl trans-N-[4-(5-bromothiazol-2-yl)cyclohexyl]carbamate (0.3 g, 864 umol, 1.0 eq.), Pd(PPh₃)₄ (100 mg, 86 umol, 0.1 eq.), Na₂CO₃ (275 mg, 3 mmol, 3.0 eq.), KF (100 mg, 2 mmol, 2.0 eq.) in toluene (2 mL)/H₂O (0.6 mL)/EtOH (2 mL) was stirred at 85° C. for 12 hrs under N₂ atmosphere and concentrated under reduced pressure to give a residue. The reaction was diluted with H₂O (80 mL) and extracted with EtOAc (100 mL×3). The combined organic layers were washed with brine (100 mL), dried over Na₂SO₄, filtered, concentrated under reduced pressure and purified by column chromatography (SiO₂, Petroleum ether:EtOAc=10:1 to 1:1) to yield isopropyl trans-N-[4-[5-(4-amino-2-hydroxy-phenyl)thiazol-2-yl]cyclohexyl]carbamate (0.1 g, crude) as a yellow solid.

ESI [M+H]=376.2

### Step 5: Synthesis of isopropyl trans-N-[4-[5-[4-amino-2-[tert-butyl(dimethyl)silyl]oxy-phenyl]thiazol-2-yl]cyclohexyl]carbamate

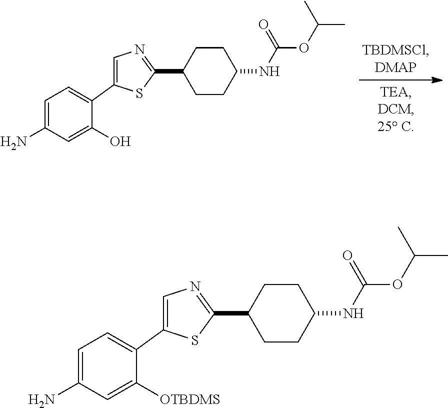 [SMILES: CC(C)OC(=O)N[C@H]1CC[C@H](c2ncc(-c3ccc(N)cc3O)s2)CC1 ; CC(C)OC(=O)N[C@H]1CC[C@H](c2ncc(-c3ccc(N)cc3O[Si](C)(C)C(C)(C)C)s2)CC1]

To a solution of isopropyl trans-N-[4-[5-(4-amino-2-hydroxy-phenyl)thiazol-2-yl]cyclohexyl]carbamate (0.1 g, 266 umol, 1.0 eq.) in DCM (5 mL) was added TEA (54 mg, 533 umol, 2.0 eq.) and DMAP (3 mg, 27 umol, 0.1 eq.), TBSCl (60 mg, 399 umol, 1.5 eq.). The mixture was stirred at 25° C. for 1 hr, quenched by H₂O (100 mL) and extracted with DCM (100 mL×3). The combined organic layers were washed with sat. aq. NaCl (100 mL), dried over Na₂SO₄, filtered, concentrated under reduced pressure and purified by prep-TLC (SiO₂, Petroleum ether:EtOAc=1:1) to yield isopropyl trans-N-[4-[5-[4-amino-2-[tert-butyl(dimethyl)silyl]oxy-phenyl]thiazol-2-yl]cyclohexyl]carbamate (70 mg, crude) as a yellow solid. ESI [M+H]=490.4

### Step 6: Synthesis of trans-(4-nitrophenyl) N-[3-[tert-butyl(dimethyl)silyl]oxy-4-[2-[4-(isopropoxycarbonylamino)cyclohexyl]thiazol-5-yl]phenyl]carbamate

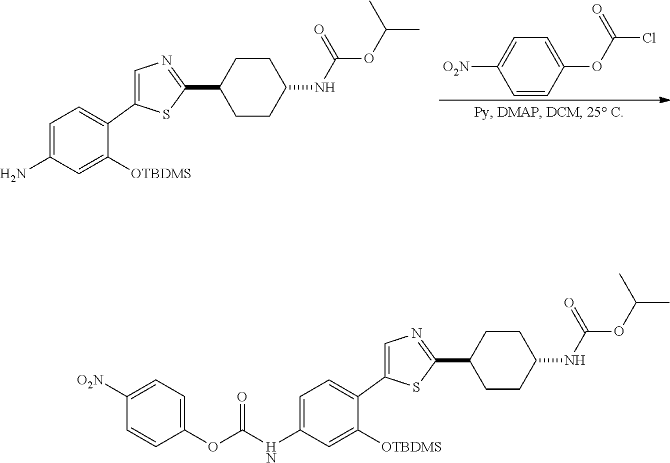 [SMILES: CC(C)OC(=O)N[C@H]1CC[C@H](c2ncc(-c3ccc(N)cc3O[Si](C)(C)C(C)(C)C)s2)CC1 ; O=C(Cl)Oc1ccc([N+](=O)[O-])cc1 ; CC(C)OC(=O)N[C@H]1CC[C@H](c2ncc(-c3ccc(NC(=O)Oc4ccc([N+](=O)[O-])cc4)cc3O[Si](C)(C)C(C)(C)C)s2)CC1]

To a solution of isopropyl trans-N-[4-[5-[4-amino-2-[tert-butyl(dimethyl)silyl]oxy-phenyl]thiazol-2-yl]cyclohexyl]carbamate (70 mg, 143 umol, 1.0 eq.) in DCM (2 mL) was added pyridine (34 mg, 429 umol, 3.0 eq.) DMAP (2 mg, 14 umol, 0.1 eq.), and (4-nitrophenyl) carbonochloridate (35 mg, 172 umol, 1.2 eq.). The mixture was stirred at 25° C. for 30 mins to yield a solution of trans-(4-nitrophenyl) N-[3-[tert-butyl(dimethyl)silyl]oxy-4-[2-[4-(isopropoxycarbonylamino)cyclohexyl]thiazol-5-yl]phenyl]carbamate (93 mg, crude) in DCM (2 mL). ESI [M+H]=655.3

### Step 7: Synthesis of isopropyl trans-N-[4-[5-[4-(benzylcarbamoylamino)-2-[tert-butyl(dimethyl)silyl]oxy-phenyl]thiazol-2-yl]cyclohexyl]carbamate

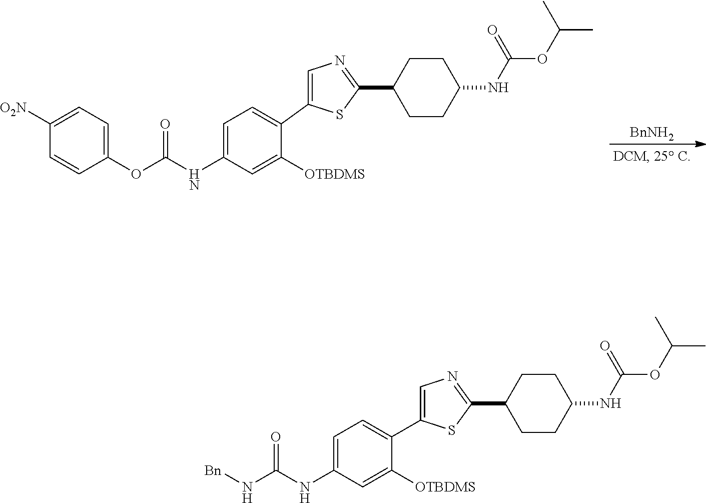

To a solution of benzylamine (61 mg, 568 umol, 4.0 eq.) in DCM (2 mL) was added the solution of trans-(4-nitrophenyl) N-[3-[tert-butyl(dimethyl)silyl]oxy-4-[2-[4-(isopropoxycarbonylamino)cyclohexyl]thiazol-5-yl]phenyl]carbamate (93 mg, 142 umol, 1.0 eq.) in DCM (2 mL). The mixture was stirred at 25° C. for 10 mins, quenched by 1N HCl (50 mL) and extracted with DCM (50 mL×3). The combined organic layers were washed with sat. aq. Na₂CO₃ (50 mL), dried over Na₂SO₄, filtered, concentrated and purified by prep-TLC (SiO₂, Petroleum ether:EtOAc=1:1) to yield isopropyl trans-N-[4-[5-[4-(benzylcarbamoylamino)-2-[tert-butyl(dimethyl)silyl]oxy-phenyl]thiazol-2-yl]cyclohexyl]carbamate (60 mg, 96 umol, 67.8% yield) as a yellow oil. ESI [M+H]=623.3

### Step 8: Synthesis of isopropyl ((trans)-4-(5-(4-(3-benzylureido)-2-hydroxy phenyl)thiazol-2-yl)cyclohexyl)carbamate (20)

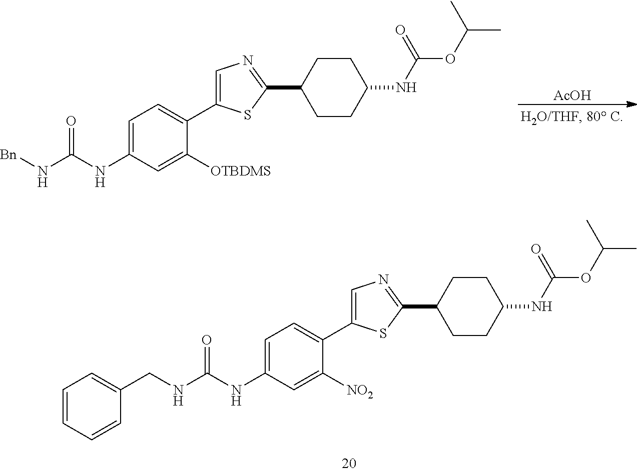

To a solution of isopropyl trans-N-[4-[5-[4-(benzylcarbamoylamino)-2-[tert-butyl(dimethyl)silyl]oxy-phenyl]thiazol-2-yl]cyclohexyl]carbamate (60 mg, 96 umol, 1.0 eq.) in H₂O (1 mL)/CH₃COOH (1 mL)/THF (1 mL). The mixture was stirred at 80° C. for 1 hr and then concentrated under reduced pressure. The reaction mixture was diluted with H₂O (20 mL) and extracted with EtOAc (30 mL×3). The combined organic layers were washed with brine (20 mL), dried over Na₂SO₄, filtered, concentrated under reduced pressure and purified by prep-HPLC (column: Phenomenex Synergi C18 100×30 mm×4 um; mobile phase: [water(TFA)-ACN]; B %: 45%-75%, 10 mins) to yield isopropyl ((trans)-4-(5-(4-(3-benzylureido)-2-hydroxy phenyl)thiazol-2-yl)cyclohexyl)carbamate (20) (16.0 mg, 31 umol, 32.3% yield, 99.2% purity) as a pale yellow solid. ¹H NMR (400 MHz, methanol-d4) δ=8.18-8.07 (m, 1H), 7.51 (d, J=8.6 Hz, 1H), 7.35 (d, J=4.3 Hz, 3H), 7.32-7.25 (m, 2H), 6.81 (dd, J=2.0, 8.5 Hz, 1H), 4.85 (br s, 1H), 4.42 (s, 2H), 3.58-3.40 (m, 1H), 3.06 (br t, J=12.0 Hz, 1H), 2.23 (br d, J=13.3 Hz, 2H), 2.13-1.96 (m, 2H), 1.86-1.66 (m, 2H), 1.57-1.32 (m, 2H), 1.25 (br d, J=6.0 Hz, 6H). ESI [M+H]=509.2

### Example 21: Preparation of isopropyl ((trans)-4-(5-(4-(3-benzylureido)-2-chlorophenyl)thiazol-2-yl)cyclohexyl)carbamate

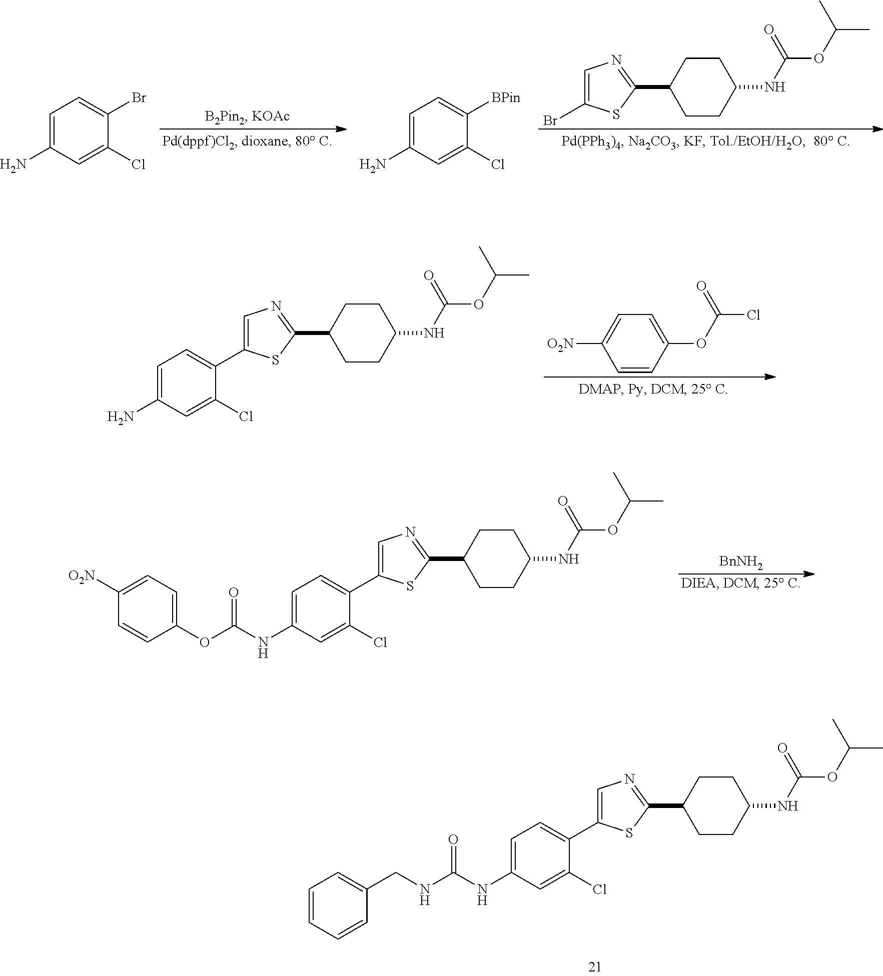

### Step 1: Synthesis of 3-chloro-4-(4,4,5,5-tetramethyl-1,3,2-dioxaborolan-2-yl)aniline

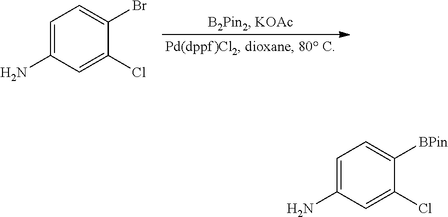

A mixture of 4,4,5,5-tetramethyl-2-(4,4,5,5-tetramethyl-1,3,2-dioxaborolan-2-yl)-1,3,2-dioxaborolane (442 mg, 1 mmol, 1.2 eq.), 4-bromo-3-chloro-aniline (300 mg, 1 mmol, 1.0 eq.), KOAc (427 mg, 4 mmol, 3.0 eq.), Pd(dppf)Cl₂ (106 mg, 145 umol, 0.1 eq.) in dioxane (10 mL) was stirred at 80° C. for 12 hrs under N₂ atmosphere, quenched by H₂O (10 mL) at 0° C. and extracted with EtOAc (100 mL×3). The organic layers were dried over Na₂SO₄, filtered, concentrated and purified by column chromatography (SiO₂, Petroleum ether:EtOAc=1:0 to 100:1) to yield 3-chloro-4-(4,4,5,5-tetramethyl-1,3,2-dioxaborolan-2-yl)aniline (280 mg, 1 mmol, 76.0% yield) as a yellow solid.

¹H NMR (CDCl₃, 400 MHz): δ=7.46 (d, J=8.2 Hz, 1H), 6.58 (d, J=2.2 Hz, 1H), 6.44 (dd, J=8.2, 2.0 Hz, 1H), 3.79 (br s, 2H), 1.27 (s, 12H). ESI [M+H]=254.3

### Step 2: Synthesis of isopropyl trans-N-[4-[5-(4-amino-2-chloro-phenyl)thiazol-2-yl]cyclohexyl]carbamate

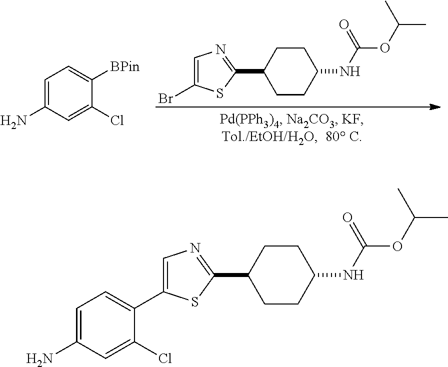

To a solution of 3-chloro-4-(4,4,5,5-tetramethyl-1,3,2-dioxaborolan-2-yl)aniline (87 mg, 345 umol, 1.2 eq.), isopropyl N-[4-(5-bromothiazol-2-yl)cyclohexyl]carbamate (100 mg, 287 umol, 1.0 eq.) in toluene (1 mL)/EtOH (1 mL)/H₂O (0.3 mL) was added KF (25 mg, 431 umol, 1.5 eq.), Na₂CO₃ (61 mg, 575 umol, 2.0 eq.), and Pd(PPh₃)₄ (33 mg, 28 umol, 0.1 eq.). The mixture was stirred at 80° C. for 12 hrs, quenched by H₂O (30 mL) at 0° C. and extracted with EtOAc (200 mL×3). The organic layers were dried over Na₂SO₄, filtered, concentrated and purified by prep-TLC (SiO₂, Petroleum ether:EtOAc=2:1) to yield isopropyl trans-N-[4-[5-(4-amino-2-chloro-phenyl)thiazol-2-yl]cyclohexyl]carbamate (17 mg, crude) as a yellow oil.

### Step 3: Synthesis of trans-(4-nitrophenyl)-N-[3-chloro-4-[2-[4-(isopropoxy carbonylamino)cyclohexyl]thiazol-5-yl]phenyl]carbamate

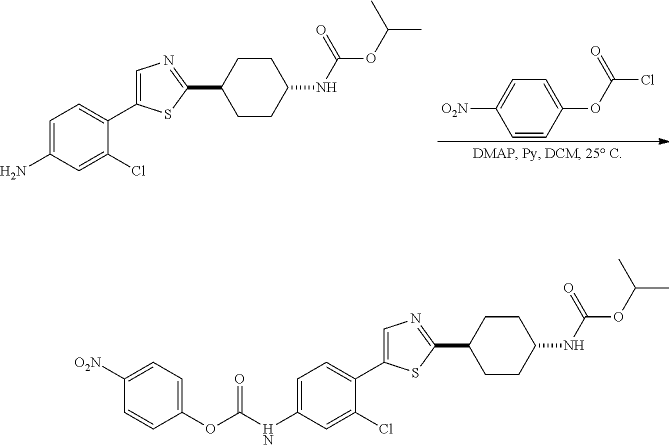

A mixture of isopropyl trans-N-[4-[5-(4-amino-2-chloro-phenyl)thiazol-2-yl]cyclohexyl]carbamate (17 mg, 43 umol, 1 eq.), (4-nitrophenyl) chloroformate (13 mg, 64 umol, 1.5 eq.), DMAP (527 ug, 4 umol, 0.1 eq.), pyridine (10 mg, 129 umol, 3.0 eq.) in DCM (1 mL) was stirred at 25° C. for 0.5 hr under N₂ atmosphere to yield trans-(4-nitrophenyl)-N-[3-chloro-4-[2-[4-(isopropoxycarbonylamino)cyclohexyl]thiazol-5-yl]phenyl]carbamate (30 mg, crude) as a yellow solution.

### Step 4: Synthesis of isopropyl ((trans)-4-(5-(4-(3-benzylureido)-2-chlorophenyl)thiazol-2-yl)cyclohexyl)carbamate (21)

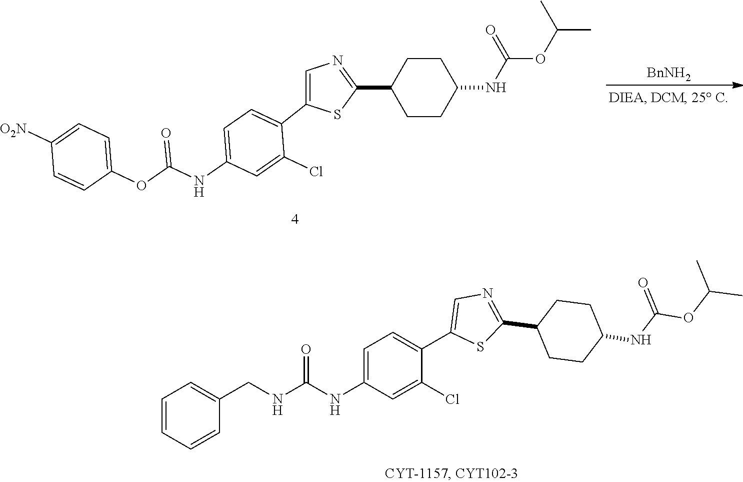

To a mixture of benzylamine (23 mg, 214 umol, 5.0 eq.), DIEA (16 mg, 128 umol, 3.0 eq.) in DCM (2 mL) was added trans-(4-nitrophenyl)-N-[3-chloro-4-[2-[4-(isopropoxy carbonylamino)cyclohexyl]thiazol-5-yl]phenyl]carbamate (24 mg, 42 umol, 1.0 eq.) in DCM (1 mL), the mixture was stirred at 25° C. for 0.5 hr under N₂ atmosphere, quenched by H₂O (5 mL) at 0° C. and extracted with DCM (50 mL×3). The organic layers were dried over Na₂SO₄, filtered, concentrated and purified by prep-HPLC (column: Luna C18 100×30 5u; mobile phase: [water(0.1% TFA)-ACN]; B %: 40%-60%, 10 mins) to isopropyl ((trans)-4-(5-(4-(3-benzylureido)-2-chlorophenyl)thiazol-2-yl)cyclohexyl) carbamate (21) (2 mg, 3.72 umol, 8.6% yield, 98.0% purity) as pale yellow solid. ¹H NMR (DMSO-d6, 400 MHz): δ=7.79-7.89 (m, 2H), 7.51 (d, J=8.4 Hz, 1H), 7.23-7.41 (m, 5H), 7.21-7.27 (m, 1H), 4.68-4.82 (m, 1H), 4.31 (s, 2H), 3.24-3.29 (m, 1H), 2.88-2.99 (m, 1H), 2.08-2.18 (m, 2H), 1.91 (br d, J=10.1 Hz, 2H), 1.49-1.63 (m, 2H), 1.27-1.40 (m, 2H), 1.17 ppm (d, J=6.1 Hz, 6H). ESI [M+H]=527.1

### Example 22: Preparation of isopropyl ((trans)-4-(5-(4-(3-benzylureido)-2-(trifluoromethyl)phenyl)thiazol-2-yl)cyclohexyl)carbamate

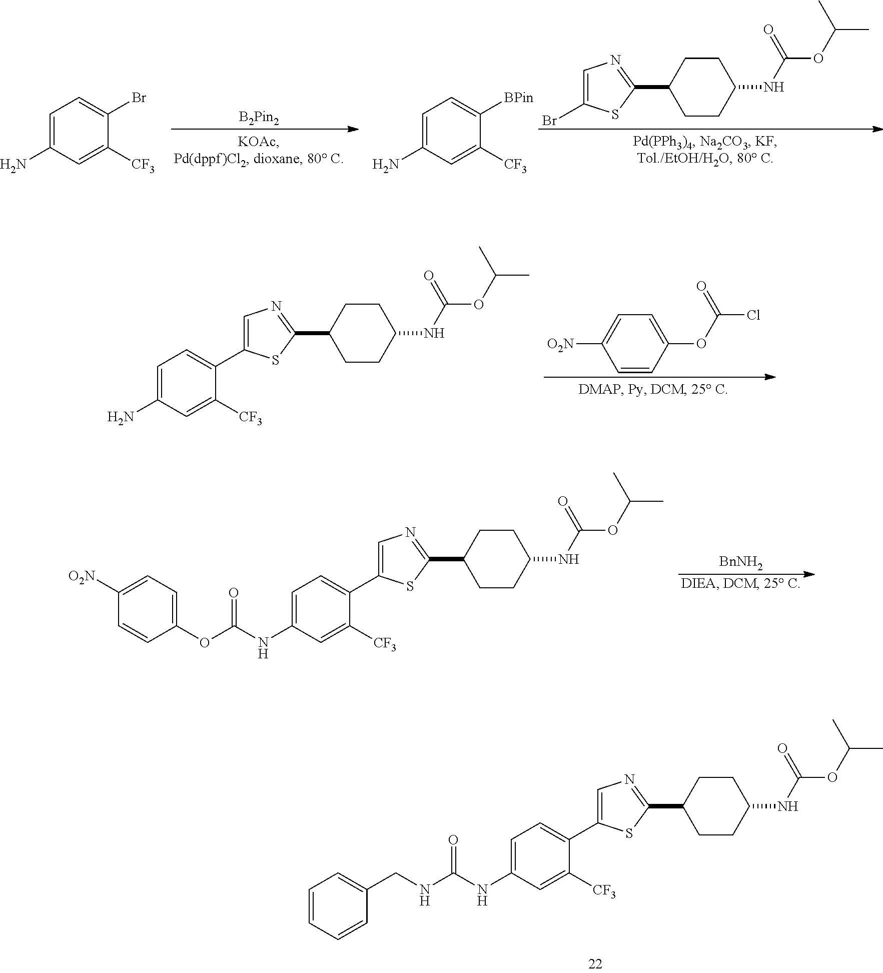

Following the same protocols and under the same conditions as for example 21, step 1-4, using 4-bromo-3-(trifluoromethyl)aniline in step 1, isopropyl ((trans)-4-(5-(4-(3-benzylureido)-2-(trifluoromethyl)phenyl)thiazol-2-yl)cyclohexyl)carbamate (22) was obtained as a pale yellow solid. ¹H NMR (400 MHz, DMSO-d6) δ=9.15 (s, 1H), 8.07 (d, J=2.0 Hz, 1H), 7.61 (dd, J=2.0, 8.4 Hz, 1H), 7.56 (s, 1H), 7.41 (d, J=8.6 Hz, 1H), 7.34-7.19 (m, 5H), 7.09 (br s, 1H), 6.85 (t, J=6.0 Hz, 1H), 4.72 (td, J=6.3, 12.6 Hz, 1H), 4.30 (d, J=6.0 Hz, 2H), 3.63-3.61 (m, 1H), 3.08 (td, J=4.3, 8.4 Hz, 1H), 2.02-1.90 (m, 2H), 1.82 (td, J=4.5, 8.9 Hz, 2H), 1.63 (br d, J=5.3 Hz, 4H), 1.14 (d, J=6.2 Hz, 6H). ESI [M+H]=561.2

### Example 23: Preparation of isopropyl ((trans)-4-(5-(2-fluoro-4-((isopropoxycarbonyl)amino)phenyl)thiazol-2-yl)cyclohexyl)carbamate

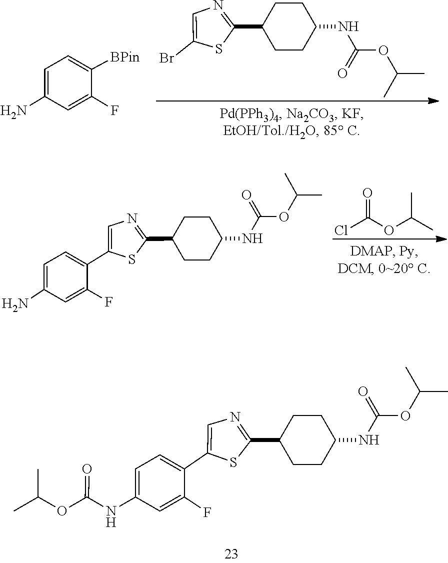

### Step 1: Synthesis of isopropyl trans-N-[4-[5-(4-amino-2-fluoro-phenyl)thiazol-2-yl]cyclohexyl]carbamate

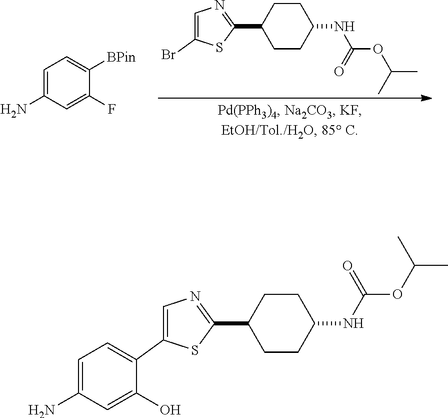

A mixture of 3-fluoro-4-(4,4,5,5-tetramethyl-1,3,2-dioxaborolan-2-yl)aniline (0.4 g, 2 mmol, 1.0 eq.), isopropyl trans-N-[4-(5-bromothiazol-2-yl)cyclohexyl]carbamate (644 mg, 2 mmol, 1.1 eq.), Na₂CO₃ (536 mg, 5 mmol, 3.0 eq.), KF (196 mg, 3 mmol, 2.0 eq.) and Pd(PPh₃)₄ (195 mg, 169 umol, 0.1 eq.) in toluene (10 mL)/H₂O (3 mL)/EtOH (10 mL) was stirred at 85° C. for 12 hrs under N₂ atmosphere, quenched by H₂O (50 mL) and extracted with EtOAc (100 mL×3). The combined organic layers were dried over Na₂SO₄, filtered, concentrated and purified by column chromatography (SiO₂, Petroleum ether:EtOAc=1:0 to 1:1) to yield isopropyl trans-N-[4-[5-(4-amino-2-fluoro-phenyl)thiazol-2-yl]cyclohexyl]carbamate (0.5 g, 1 mmol, 70.7% yield) as a yellow oil.

ESI [M+H]=378.1

### Step 2: Synthesis of isopropyl ((trans)-4-(5-(2-fluoro-4-((isopropoxycarbonyl)amino)phenyl)thiazol-2-yl)cyclohexyl)carbamate (23)

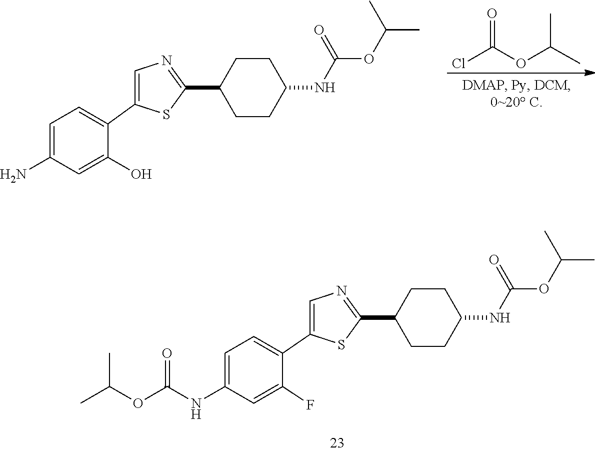

To a solution of isopropyl trans-N-[4-[5-(4-amino-2-fluoro-phenyl)thiazol-2-yl]cyclohexyl]carbamate (40 mg, 106 umol, 1.0 eq.) in DCM (2 mL) was added pyridine (42 mg, 530 umol, 5.0 eq.) and DMAP (1 mg, 11 umol, 0.1 eq.), then isopropyl chloroformate (16 mg, 127 umol, 1.2 eq.) was added batch at 0° C. The mixture was stirred at 20° C. for 0.5 hr, quenched by 1N HCl (30 mL) and extracted with DCM (50 mL×3). The organic layers were washed with sat. aq. Na₂CO₃ (30 mL), dried over Na₂SO₄, filtered, concentrated and purified by prep-HPLC (column: Nano-Micro UniSil 5-100 C18 ULTRA 100×250 mm 5 um; mobile phase: [water(0.1% TFA)-ACN]; B %: 50%-70%, 10 mins) to yield isopropyl ((trans)-4-(5-(2-fluoro-4-((isopropoxycarbonyl)amino)phenyl)thiazol-2-yl)cyclohexyl)carbamate (23) (16 mg, 35 umol, 32.7% yield, 99.0% purity) as a white solid. ¹H NMR (400 MHz, methanol-d4) δ=7.92 (s, 1H), 7.61-7.46 (m, 2H), 7.21 (br d, J=7.5 Hz, 1H), 5.03-4.93 (m, 1H), 4.85-4.75 (m, 1H), 3.51-3.37 (m, 1H), 2.99 (br t, J=12.1 Hz, 1H), 2.20 (br d, J=11.9 Hz, 2H), 2.06 (br d, J=11.7 Hz, 2H), 1.68 (q, J=12.9 Hz, 2H), 1.46-1.35 (m, 2H), 1.30 (d, J=6.2 Hz, 6H), 1.22 (br d, J=6.0 Hz, 6H). ESI [M+H]=464.1

### Example 24. Biological Activity of the Compounds of the Present Disclosure

The biological activity of the compounds of the present disclosure was determined utilizing the assays described herein.

### Intracellular Lactate Accumulation Assay

Intracellular lactate accumulation after a 2 h treatment with compounds of the present invention was measured in Daudi (Burkitt Lymphoma) cells using the Lactate-Glo Assay (Promega).

### Procedure

Daudi cells were seeded at 7,500 cells per well in 150 μL of growth medium (RPMI-1640 with 2 mM GlutaMAX+10% FBS) in clear, 96-well, flat-bottom microplates, and incubated overnight (37° C., 5% CO₂). Compounds of the present invention were added using an INTEGRA VIAFLO 96 (8 point, 3-fold serial dilution, top concentration 1, 10, or 20 μM, 0.4% final DMSO concentration). After a 2 h incubation (37° C., 5% CO₂) microplates containing cells were washed twice with 150 μL ice-cold PBS, resuspended in 25 μL ice-cold PBS containing 12.5 μL 0.6N HCl, and placed on a microplate shaker for 5 min to lyse the cells. Following lysis, 12.5 μL 1M Trizma base was added to neutralize the suspension, and microplates were returned to the microplate shaker for 1 min. Plates were sealed and stored at −20° C. until further processing. On the day of the assay, microplates were thawed for 45 min at room temperate and 50 L Lactate Detection Reagent (prepared as specified by manufacturer) was added to each well. Microplates were placed on a plate shaker for 1 min and incubated at room temperature for 1 h. Luminescence was measured using a BioTek Cytation 5 Multi-Mode Reader. Curve fitting and calculation of EC₅₀ was performed using GraphPad Prism.

### MCT1 Transport Inhibition Assay

Inhibition of MCT1 transport after treatment with compounds of the present invention was measured in MDCK-II cells that overexpress human MCT1 by quantifying the transport of 2-Thiophene-glycoxylic acid (TPGA) into cells using LC-MS/MS.

### Procedure

MDCK-II cells are seeded at 60,000 cells per well in growth medium (low-glucose DMEM+10% FBS) in 96-well trans-well membrane plates, and incubated overnight (37° C., 5% CO₂). Cells were co-transfected with mammalian expression constructs coding for MCT1 and CD147 at a 2:1 ratio or an empty vector control (GFP) and then incubated for 48 h (37° C., 5% CO₂). Cells were washed 3 times with HBSS and cells were preincubated with inhibitors or vehicle control at room temperature in HBSS for 30 min with orbital shaking (60 rpm). HBSS was aspirated from the wells and replaced with HBSS with 25 mM Bis-Tris pH5.5+inhibitor or vehicle control+500 μM TPGA and incubated for 1 min at room temperature with orbital shaking (60 rpm). Both the apical and basolateral side of the trans-well insert were washed 4 times with ice-cold PBS. Cells were lysed with 60 μL of cell extraction solution, and the amount of TPGA in each well was quantified in triplicate by LC-MS/MS. The MCT1-mediated uptake rate was calculated using the following equation:

$$\frac{\begin{matrix}MCT 1 - mediated uptake rate ( p mol / min / {cm}^{2} ) = \\ \begin{matrix}( Cellular accumulation in cells expressing MCT 1 ) - \\ ( Mean cellular accumulation in control cells )\end{matrix}\end{matrix}}{Surface area \times Incubation time}$$

Percent inhibition for each concentration of inhibitor tested was calculated using the following equation:

$$Percent inhibition = 100 - 100 \times \frac{( MCT 1 - mediated uptake rate ) \text{?}}{{( MCT 1 - mediated uptake rate )}_{vehicle control}}$$

$$\text{?} \text{indicates text missing or illegible when filed}$$

Curves were fit to the calculated percent inhibition for each inhibitor, and the IC₅₀ representing the concentration of inhibitor at which MCT1 is inhibited by 50% was calculated from this curve.

The biological activity of the compounds of the present application measured by the described assays above are shown in Table A below for LactateGlo EC₅₀ (“A” means <50 nM; “B” means ≥50 nM and <150 nM; “C” means ≥150 nM and <1000 nM; “D” means ≥1000 nM and <5000 nM; “E” means >5000 nM) and BioIVT MCT1 Transporter IC₅₀ (“+++++” means <50 nM; “++++” means ≥50 nM and <150 nM; “+++” means ≥150 nM and <1000 nM; “++” means ≥1000 nM and <5000 nM; “+” means >5000 nM). N/D=Not Determined.

TABLE A

| | | | BioIVT |
|---|---|---|---|
| | | LactateGlo | MCT1 |
| Ex. | | EC₅₀ | Transporter |
| # | Structure | (nM) | IC₅₀ (nM) |
| 14 | 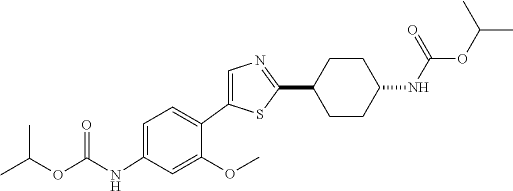 | C | N/D |
| 19 | 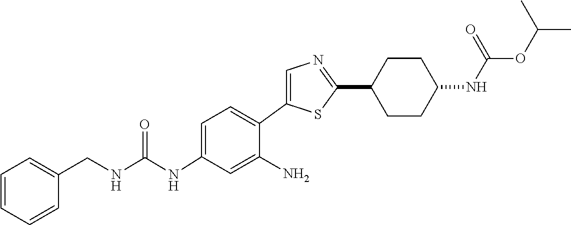 | A | N/D |
| 20 | 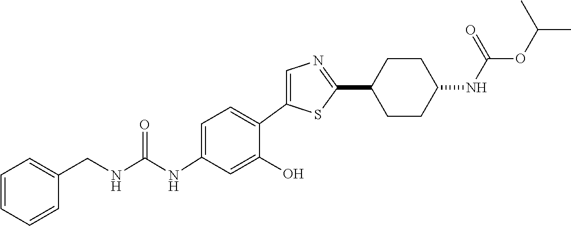 | A | N/D |
| 23 | 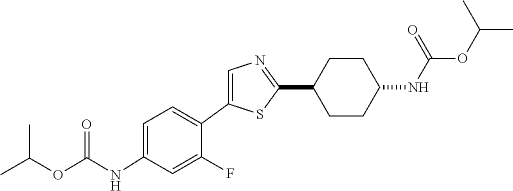 | E | N/D |
| 32 | 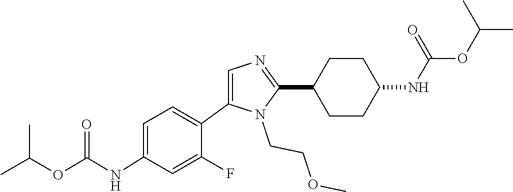 | N/D | + |
| | 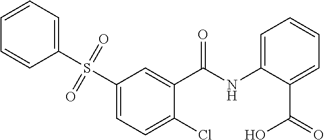 [SMILES: O=C(Nc1ccccc1C(=O)O)c1cc(S(=O)(=O)c2ccccc2)ccc1Cl] | N/D | 73 |
| | BAY8002 (CAS No. 724440-27-1) | | |
| | 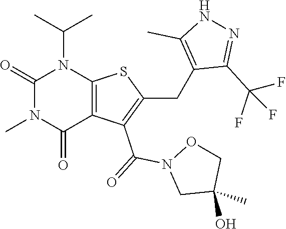 | N/D | 190 |
| | AZD3965 (CAS No. 1448671-31-5) | | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 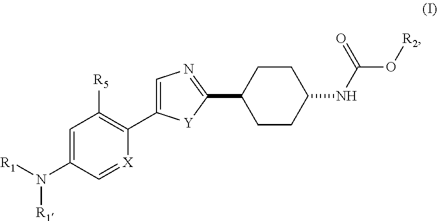
or a pharmaceutically acceptable prodrug, solvate, or salt thereof, wherein:
X is CR₃ or N;
Y is S or NR₄;
R₁ is H, —C(O)—R₁ₐ, —C(NH)—R₁ₐ, C₁-C₆ alkyl, C₂-C₆ alkenyl, C₂-C₆ alkynyl, C₆-C₁₀ aryl, or 5- to 10-membered heteroaryl;
R₁ is H or C₁-C₆ alkyl;
R₁ₐ is —OH, —O(C₁-C₆ alkyl), —NH₂, —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl, wherein the —O(C₁-C₆ alkyl), —NH(C₁-C₆ alkyl), —N(C₁-C₆ alkyl)₂, C₁-C₆ alkyl, C₂-C₆ alkenyl, C₂-C₆ alkynyl, or C₁-C₆ haloalkyl is optionally substituted with one or more R_{1b};
R_{1b} is C₆-C₁₀ aryl or 5- to 10-membered heteroaryl, wherein the C₆-C₁₀ aryl or 5- to 10-membered heteroaryl is optionally substituted with one or more halogen, —CN, —OH, —O(C₁-C₆ alkyl), —NH₂, —NH(C₁-C₆ alkyl), —N(C₁-C₆ alkyl)₂, C₁-C₆ alkyl, C₂-C₆ alkenyl, or C₂-C₆ alkynyl;
R₂ is C₁-C₆ alkyl, C₂-C₆ alkenyl, C₂-C₆ alkynyl, C₃-C₁₀ cycloalkyl, or 3- to 10-membered heterocyclyl;
R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), —NHC(O)—N(C₁-C₆ alkyl)₂, C₁-C₆ alkyl, C₂-C₆ alkenyl, C₂-C₆ alkynyl, C₃-C₁₀ cycloalkyl, or 3- to 10-membered heterocyclyl is optionally substituted with one or more R₃ₐ;
R₃ₐ is halogen, —OH, —O(C₁-C₆ alkyl), —NH₂, —NH(C₁-C₆ alkyl), —N(C₁-C₆ alkyl)₂, —N(C₁-C₆alkyl)-SO₂—(C₁-C₆ alkyl), —NHC(O)—(C₁-C₆ alkyl), —NHC(O)—NH₂, —NHC(O)—NH(C₁-C₆ alkyl), —NHC(O)—N(C₁-C₆ alkyl)₂, C₃-C₁₀ cycloalkyl, or 3- to 10-membered heterocyclyl;
R₄ is C₁-C₆ alkyl optionally substituted with one or more R₄ₐ;
R₄ₐ is —OH, —O(C₁-C₆ alkyl), —NH₂, —NH(C₁-C₆ alkyl), —N(C₁-C₆ alkyl)₂, or —SO₂—(C₁-C₆ alkyl); and
R₅ is H or halogen.

2. The compound of claim 1, wherein:
X is CR₃ or N;
Y is S or NR₄;
R₁ is H, —C(O)—R₁ₐ, —C(NH)—R₁ₐ, C₁-C₆ alkyl, or 5- to 10-membered heteroaryl;
R₁ₐ is —O(C₁-C₆ alkyl), —NH₂, —NH(C₁-C₆ alkyl), C₁-C₆ alkyl, or C₁-C₆ haloalkyl, wherein the —O(C₁-C₆ alkyl), —NH(C₁-C₆ alkyl), C₁-C₆ alkyl, or C₁-C₆ haloalkyl is optionally substituted with one or more R_{1b};
R_{1b} is C₆-C₁₀ aryl or 5- to 10-membered heteroaryl, wherein the C₆-C₁₀ aryl or 5- to 10-membered heteroaryl is optionally substituted with one or more halogen;
R₂ is C₁-C₆ alkyl;
R₃ is H, halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), C₁-C₆ alkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), C₁-C₆ alkyl, or 3- to 10-membered heterocyclyl is optionally substituted with one or more R₃ₐ;
R₃ₐ is halogen, —OH, —O(C₁-C₆ alkyl), —N(C₁-C₆ alkyl)₂, —N(C₁-C₆ alkyl)-S02-(C₁-C₆ alkyl), or 3- to 10-membered heterocyclyl; and
R₄ is C₁-C₆ alkyl optionally substituted with one or more R₄ₐ.
R₄ₐ is —OH, —O(C₁-C₆ alkyl), or —SO₂—(C₁-C₆ alkyl).

3. The compound of claim 1 or 2, wherein X is CR₃.

4. The compound of claim 1 or 2, wherein X is N.

5. The compound of any one of claims 1-4, wherein Y is S.

6. The compound of any one of claims 1-4, wherein Y is NR₄.

7. The compound of any one of claims 1-6, wherein R₁ is H.

8. The compound of any one of claims 1-6, wherein R₁ is —C(O)—R₁ₐ, —C(NH)—R₁ₐ, C₁-C₆ alkyl, or 5- to 10-membered heteroaryl.

9. The compound of any one of claims 1-8, wherein R_{1′} is H.

10. The compound of any one of claims 1-8, wherein R_{1′} is C₁-C₆ alkyl.

11. The compound of any one of claims 1-6 and 8-10, wherein R₁ₐ is —O(C₁-C₆ alkyl), —NH₂, —NH(C₁-C₆ alkyl), C₁-C₆ alkyl, or C₁-C₆ haloalkyl, wherein the —O(C₁-C₆ alkyl), —NH(C₁-C₆ alkyl), C₁-C₆ alkyl, or C₁-C₆ haloalkyl is optionally substituted with one or more R_{1b}.

12. The compound of any one of claims 1-6 and 8-11, wherein R_{1b} is C₆-C₁₀ aryl or 5- to 10-membered heteroaryl, wherein the C₆-C₁₀ aryl or 5- to 10-membered heteroaryl is optionally substituted with one or more halogen.

13. The compound of claim 12, wherein R_{1b} is phenyl.

14. The compound of any one of claims 1-13, wherein R₂ is C₁-C₆ alkyl.

15. The compound of claim 14, wherein R₂ is isopropyl.

16. The compound of any one of claims 1-3 and 5-15, wherein R₃ is H.

17. The compound of any one of claims 1-3 and 5-15, wherein R₃ is halogen, —CN, —OH, —O(C₁-C₆ alkyl), —NH₂, —NH(C₁-C₆ alkyl), —N(C₁-C₆ alkyl)₂, —NHC(O)—(C₁-C₆ alkyl), —NHC(O)—NH₂, —NHC(O)—NH(C₁-C₆ alkyl), C₁-C₆ alkyl, or 3- to 10-membered heterocyclyl, wherein the —O(C₁-C₆ alkyl), —NH(C₁-C₆ alkyl), —N(C₁-C₆ alkyl)₂, —NHC(O)—(C₁-C₆ alkyl), —NHC(O)—NH(C₁-C₆ alkyl), C₁-C₆ alkyl, or 3- to 10-membered heterocyclyl, is optionally substituted with one or more R₃ₐ.

18. The compound of any one of claims 1-3, 5-15, and 17, wherein R₃ₐ is halogen, —OH, —O(C₁-C₆ alkyl), —N(C₁-C₆ alkyl)₂, —N(C₁-C₆ alkyl)-S02-(C₁-C₆ alkyl), or 3- to 10-membered heterocyclyl.

19. The compound of any one of claims 1-4 and 6-18, wherein R₄ is C₁-C₆ alkyl substituted with one or more R₄ₐ.

20. The compound of any one of claims 1-4 and 6-19, wherein R₄ₐ is —OH, —O(C₁-C₆alkyl), or —SO₂—(C₁-C₆ alkyl).

21. The compound of any one of claims 1-20, wherein R₅ is H.

22. The compound of any one of claims 1-20, wherein R₅ is halogen.

23. The compound of claim 1, wherein the compound is of Formula (Ia): 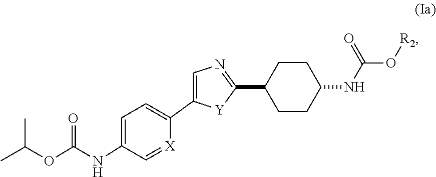
or a pharmaceutically acceptable prodrug, solvate, or salt thereof.

24. The compound of claim 1, wherein the compound is of Formula (Ib): 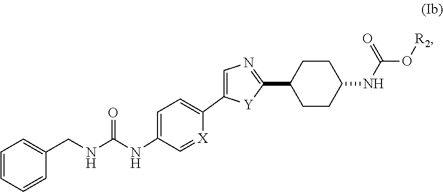
or a pharmaceutically acceptable prodrug, solvate, or salt thereof.

25. The compound of claim 1, wherein the compound is of Formula (Ic): 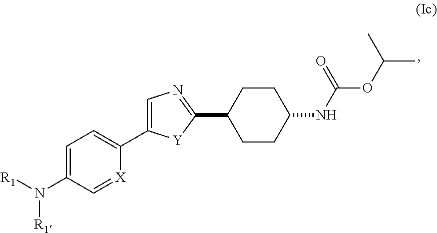
or a pharmaceutically acceptable prodrug, solvate, or salt thereof.

26. The compound of claim 1, wherein the compound is of Formula (Id): 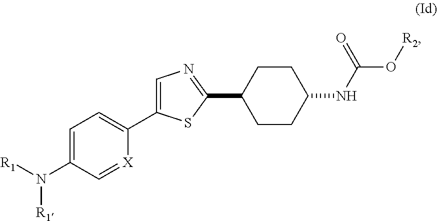
or a pharmaceutically acceptable prodrug, solvate, or salt thereof.

27. The compound of claim 1, wherein the compound is of Formula (Ie): 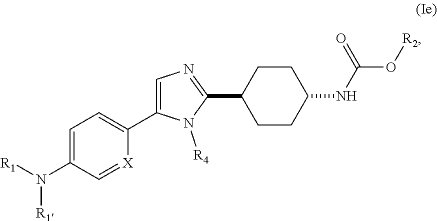
or a pharmaceutically acceptable prodrug, solvate, or salt thereof.

28. The compound of claim 1, wherein the compound is of Formula (If): 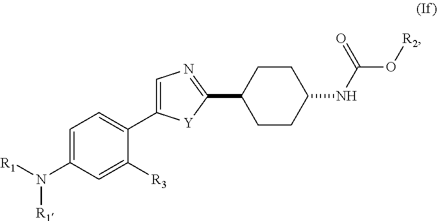
or a pharmaceutically acceptable prodrug, solvate, or salt thereof.

29. The compound of claim 1, wherein the compound is of Formula (Ig): 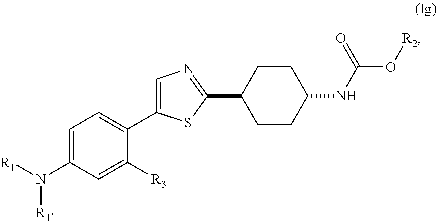
or a pharmaceutically acceptable prodrug, solvate, or salt thereof.

30. The compound of claim 1, wherein the compound is selected from a compound of Table 1, or a pharmaceutically acceptable prodrug, solvate, or salt thereof.

31. A pharmaceutical composition comprising the compound or a pharmaceutically acceptable prodrug, solvate, or salt thereof of any one of the preceding claims and a pharmaceutically acceptable diluent or carrier.

32. A method of treating or preventing a disease or disorder in a subject in need thereof, comprising administering to the subject a compound or a pharmaceutically acceptable prodrug, solvate, or salt thereof of any one of claims 1-30, or the pharmaceutical composition of claim 31.

33. The compound or a pharmaceutically acceptable prodrug, solvate, or salt thereof of any one of claims 1-30, or the pharmaceutical composition of claim 31, for use in treating or preventing a disease or disorder.

34. Use of the compound or a pharmaceutically acceptable prodrug, solvate, or salt thereof of any one of claims 1-30, or the pharmaceutical composition of claim 31, in the manufacture of a medicament for treating or preventing a disease or disorder.

35. Use of the compound or a pharmaceutically acceptable prodrug, solvate, or salt thereof of any one of claims 1-30, or the pharmaceutical composition of claim 31, for treating or preventing a disease or disorder.

36. The method, compound, pharmaceutical composition, or use of any one of claims 32-35, wherein the disease or disorder is a cancer or a neurodegenerative disease.

37. The method, compound, pharmaceutical composition, or use of claim 36, wherein the cancer is lymphoma, leukemia, or a plasma cell neoplasm.

38. The method, compound, pharmaceutical composition, or use of claim 36, wherein the cancer is lymphoma.

39. The method, compound, pharmaceutical composition, or use of claim 38, wherein the lymphoma is a B-cell lymphoma.

40. The method, compound, pharmaceutical composition, or use of claim 39, wherein the B-cell lymphoma is diffuse large B-cell lymphoma.

41. The method, compound, pharmaceutical composition, or use of claim 38, wherein the lymphoma is mantle cell lymphoma.

42. A method of treating or preventing a disease or disorder associated with the abnormal expression or activity of monocarboxylate transporters (MCTs), or dependency on the expression or activity of at least one MCT, wherein the method comprises administering to a subject in need thereof the compound or a pharmaceutically acceptable prodrug, solvate, or salt thereof of any one of claims 1-30, or the pharmaceutical composition of claim 31.

43. A method of treating or preventing a disease or disorder, wherein the method comprises administering to a subject in need thereof the compound or a pharmaceutically acceptable prodrug, solvate, or salt thereof of any one of claims 1-30, or the pharmaceutical composition of claim 31, and
wherein the compound or pharmaceutically acceptable prodrug, solvate, or salt thereof of any one of claims 1-30, or the pharmaceutical composition of claim 31 is administered in a therapeutically effective amount to modulate the activity of monocarboxylate transporters (MCTs).

44. A method of treating or preventing a disease or disorder, wherein the method comprises:
a. identifying a subject with an abnormal expression or activity of at least one MCT, or dependency on the expression or activity of at least one MCT; and
b. administering to the subject the compound or a pharmaceutically acceptable prodrug, solvate, or salt thereof of any one of claims 1-30, or the pharmaceutical composition of claim 31.

45. Use of the compound or a pharmaceutically acceptable prodrug, solvate, or salt thereof of any one of claims 1-30, or the pharmaceutical composition of claim 31, in the manufacture of a medicament for treating or preventing a disease or disorder associated with the abnormal expression or activity of monocarboxylate transporters (MCTs), or dependency on the expression or activity of at least one MCT.

46. Use of the compound or a pharmaceutically acceptable prodrug, solvate, or salt thereof of any one of claims 1-30, or the pharmaceutical composition of claim 31, in the manufacture of a medicament for treating or preventing a disease or disorder, and
wherein the compound or pharmaceutically acceptable prodrug, solvate, or salt thereof of any one of claims 1-30, or the pharmaceutical composition of claim 31 is administered in a therapeutically effective amount to modulate the activity of monocarboxylate transporters (MCTs).

47. Use of the compound or a pharmaceutically acceptable prodrug, solvate, or salt thereof of any one of claims 1-30, or the pharmaceutical composition thereof of claim 31, in the manufacture of a medicament for treating or preventing a disease or disorder comprising:
a. identifying a subject with an abnormal expression or activity of at least one MCT, or dependency on the expression or activity of at least one MCT; and
b. administering to the subject the compound or pharmaceutically acceptable prodrug, solvate, or salt thereof of any one of claims 1-30, or the pharmaceutical composition of claim 31.

48. Use of the compound or a pharmaceutically acceptable prodrug, solvate, or salt thereof of any one of claims 1-30, or the pharmaceutical composition of claim 31, for treating or preventing a disease or disorder associated with the abnormal expression or activity of monocarboxylate transporters (MCTs), or dependency on the expression or activity of at least one MCT.

49. Use of the compound or a pharmaceutically acceptable prodrug, solvate, or salt thereof of any one of claims 1-30, or the pharmaceutical composition of claim 31, for treating or preventing a disease or disorder, and
wherein the compound is administered in a therapeutically effective amount to modulate the activity of monocarboxylate transporters (MCTs).

50. Use of the compound or a pharmaceutically acceptable prodrug, solvate, or salt thereof of any one of claims 1-30, or the pharmaceutical composition of claim 31, for treating or preventing a disease or disorder comprising:
a. identifying a subject with an abnormal expression or activity of at least one MCT, or dependency on the expression or activity of at least one MCT; and
b. administering to the subject the compound or pharmaceutically acceptable prodrug, solvate, or salt thereof of any one of claims 1-30, or the pharmaceutical composition of claim 31.

51. The compound or a pharmaceutically acceptable prodrug, solvate, or salt thereof of any one of claims 1-30, or the pharmaceutical composition of claim 31, for use in treating or preventing a disease or disorder associated with the abnormal expression or activity of monocarboxylate transporters (MCTs), or dependency on the expression or activity of at least one MCT.

52. The compound or a pharmaceutically acceptable prodrug, solvate, or salt thereof of any one of claims 1-30, or the pharmaceutical composition of claim 31, for use in treating or preventing a disease or disorder, and
wherein the compound is administered in a therapeutically effective amount to modulate the activity of monocarboxylate transporters (MCTs).

53. The compound or a pharmaceutically acceptable prodrug, solvate, or salt thereof of any one of claims 1-30, or the pharmaceutical composition of claim 31, for use in treating or preventing a disease or disorder comprising:
a. identifying a subject with an abnormal expression or activity of at least one MCT, or dependency on the expression or activity of at least one MCT; and
b. administering to the subject the compound or pharmaceutically acceptable prodrug, solvate, or salt thereof of any one of claims 1-30, or the pharmaceutical composition of claim 31.

54. The method, compound, pharmaceutical composition, or use of any one of claims 42 to 53, wherein the MCT is MCT1.

55. The method, compound, pharmaceutical composition, or use of any one of claims 42 to 53, wherein the MCT is MCT4.

56. The method, compound, pharmaceutical composition, or use of any one of claims 42 to 55, wherein the expression or activity of the MCT is increased.

57. The method, compound, pharmaceutical composition, or use of any one of claims 42 to 55, wherein the expression or activity of the MCT is decreased.

58. The method, compound, pharmaceutical composition, or use of claim 54, wherein the expression or activity of MCT1 is increased.

59. The method, compound, pharmaceutical composition, or use of claim 55, wherein the expression or activity of MCT4 is decreased.

60. The method, compound, pharmaceutical composition, or use of any one of claims 42 to 59, wherein the MCT activity of the compounds of Formula I is assessed using a lactate transporter assay.

61. The method, compound, pharmaceutical composition, or use of any one of claims 42 to 60, wherein the disease or disorder is cancer.

62. The method, compound, pharmaceutical composition, or use of claim 61, wherein the cancer is a MCT1 high-expressing cancer.

63. The method, compound, pharmaceutical composition, or use of claim 61 or claim 62, wherein the cancer is a lymphoma, a myeloma, or a solid tumor.

64. The method, compound, pharmaceutical composition, or use of any one of claims 42 to 63, wherein the compound of Formula I inhibits the activity of MCT1 with an IC₅₀ from about 5 nM to about 1000 nM.
